(19) United States
(12) Patent Application Publication
(10) Pub. No.: US 2024/0277778 A1
(43) Pub. Date: Aug. 22, 2024

(54) COMPOSITIONS AND METHODS FOR DETERMINING RESPONSIVENESS TO IMMUNE CHECKPOINT INHIBITORS (ICI), INCREASING EFFECTIVENESS OF ICI, AND TREATING CANCER

(71) Applicants: UNIVERSITY OF PITTSBURGH-OF THE COMMONWEALTH SYSTEM OF HIGHER EDUCATION, Pittsburgh, PA (US); THE UNITED STATES OF AMERICA, AS REPRESENTED BY THE SECRETARY, DEPARTMENT OF HEALTH AND HUMAN, Bethesda, MD (US)

(72) Inventors: Giorgio TRINCHIERI, Bethesda, MD (US); Amiran Kasanovich DZUTSEV, Bethesda, MD (US); John Anthony MCCULLOCH, Bethesda, MD (US); Richard R. RODRIGUES, Frederick, MD (US); Diwakar DAVAR, Pittsburgh, PA (US); Hassane Mohamed ZAROUR, Pittsburgh, PA (US)

(21) Appl. No.: 18/568,661

(22) PCT Filed: Jun. 9, 2022
(86) PCT No.: PCT/US2022/032917

§ 371 Date: Dec. 8, 2023

Related U.S. Application Data
(60) Provisional application No. 63/208,719, filed on Jun. 9, 2021.

(51) Int. Cl. A61K 35/741; A61K 39/00; C07K 16/28; C12Q 1/04

(52) U.S. Cl. CPC A61K 35/741; C07K 16/2818; C12Q 1/04; A61K 2039/505; C07K 2317/76; G01N 2800/52

(57) ABSTRACT

Disclosed herein are methods for treating cancer by administering an immune checkpoint inhibitor and modifying an amount of one or more bacteria in an intestine of the subject.

### CROSS-REFERENCE TO RELATED APPLICATIONS

This application claims the benefit of U.S. Provisional Application No. 63/208,719, filed Jun. 9, 2021, which is expressly incorporated herein by reference.

### STATEMENT REGARDING FEDERALLY SPONSORED RESEARCH OR DEVELOPMENT

This invention was made with government support under CA222203 awarded by the National Institutes of Health (NIH). The government has certain rights in this invention.

### BACKGROUND OF THE INVENTION

Immune checkpoint blockade (ICB) has transformed current treatment of melanoma and other solid tumors, providing durable clinical benefit to cancer patients. However, the majority of cancer patients fail to respond to ICB, supporting the need to identify predictive biomarkers of response, and to develop novel therapies to overcome the mechanisms of resistance to ICB. Multiple predictive biomarkers to ICB have been reported so far, including pre-existing CD8+ tumor-infiltrating T cells (Tumeh, P. C., et al. 2014; Giraldo, N. A., et al. 2017; Ballot, E. et al., 2020; Terranova-Barberio, M., et al. 2020), PD-L1 expression (Taube, J. M., et al. 2014; Herbst, R. S., et al. 2014) tumor mutation burden (Cristescu, R., et al. 2018; Rizvi, H., et al. 2018; Samstein, R. M., et al. 2019; Rizvi, N. A., et al. 2015), and early-on treatment changes in circulating CD8 T cell dynamics (Huang, A. C., et al. 2019; Huang, A. C., et al. 2017; Fairfax, B. P., et al. 2020; Valpione, S., et al. 2020; Wu, T. D., et al. 2020). Composition of the intestinal microbiome has recently emerged as a major tumor-extrinsic predictive biomarker to ICB (Sivan, A., et al. 2015; Vetizou, M., et al. 2015; Dzutsev, A., et al. 215; Gopalakrishnan, V., et al. 2018; Matson, V., et al. 2018; Routy, B., et al. 2018). In mice and in humans, composition of the intestinal microbiome modulates therapeutic activity of ICB (Gopalakrishnan, V., et al. 2018; Matson, V., et al. 2018; Routy, B., et al. 2018). The administration of certain gut commensals promotes efficacy of anti-PD-1 therapy (anti-PD-1) in melanoma-bearing mice providing evidence that the microbiota composition can drive immunotherapy outcomes (Gopalakrishnan, V., et al. 2018; Matson, V., et al. 2018; Routy, B., et al. 2018). This has been confirmed in clinical studies showing that responder-derived fecal microbiota transplantation (FMT) overcomes PD-1 resistance in patients with advanced melanoma (Baruch, E. N., et al. 2021; Davar, D., et al. 2021).

Although the association of a beneficial gut microbiome with response to anti-PD-1 in cancer patients has been reported in multiple studies (Sivan, A., et al. 2015; Vetizou, M., et al. 2015; Dzutsev, A., et al. 215; Gopalakrishnan, V., et al. 2018; Matson, V., et al. 2018; Routy, B., et al. 2018), its precise composition is not yet fully understood. In patients with advanced melanoma, key bacterial species belonging to various phyla including Actinobacteria (Bifidobacteriaceae spp., Coriobacteriaceae spp.) and Firmicutes (Ruminococcaceae spp., Lachnospiraceae spp.) have been reported to be associated with favorable outcome to anti-PD-1. Strikingly, there is limited concordance among species identified across different studies, which included a small number of patients and used different analytical approaches, not always adjusted for multiple testing (Gopalakrishnan, V., et al. 2018; Matson, V., et al. 218; Frankel, A. E., et al. 2017; Peters, B. A., et al. 219). Three studies have reported the meta-analysis of shotgun and 16S RNA sequencing of published cohorts including multiple histologies (melanoma, renal and non-small cell lung cancers) treated with either anti-PD-1 or anti-CTLA-4 or both, using uniform computational approaches (Shaikh, F. Y., et al. 2021; Gharaibeh, R. Z. & Jobin, C 2019; Limeta, A., et al., 2020). Separately, there is evidence that gut microbiome composition is also associated with the occurrence of immune related adverse events (irAEs) following ICB. In melanoma patients treated with anti-CTLA-4, baseline gut microbiota enriched in Firmicutes and lacking in Bacteriodetes was associated with improved clinical outcome and higher occurrence of ICB-induced colitis (Chaput, N., et al. 2017; Dubin, K., et al. 2016). FMT from healthy donors resolved colitis in two melanoma patients treated with anti-CTLA-4 and anti-PD-1 (Wang, Y., et al. 2018). Whether specific intestinal microbiota signatures can reliably predict clinical outcome and irAEs in PD-1 treated melanoma patients remains to be ascertained (Shaikh, F. Y., et al. 2021; Gharaibeh, R. Z. & Jobin, C 2019; Limeta, A., et al., 2020). In addition, the specific combination of bacteria for increasing effectiveness of an immune checkpoint inhibitor remains to be explored. The compositions and methods disclosed herein address these and other needs.

### SUMMARY OF THE INVENTION

To address these questions, the study described herein performed an in-depth analysis of largest-to-date novel cohort of microbiome samples of PD-1 treated melanoma patients integrating response and time-to-event outcomes along with patient-level variables including medication intake, markers of systemic inflammation such as neutrophil-lymphocyte ratio (NLR), and irAE development. It was observed that the baseline intestinal microbiota composition reflected maximal separation between the clinical outcomes at 9-10 months and identified key taxonomic clades with shared functional characteristics that are associated with response and non-response to anti-PD-1. It was observed that adverse clades were characterized by high levels of lipopolysaccharide (LPS)-producing genes that were associated with an interleukin 8 (TL-8) and tumor necrosis factor (TNF) mediated inflammatory program; and transkingdom network analyses indicated that these changes were governed by detrimental microbiota. In addition, this study identified two intestinal microbial signatures differentially associated with development of irAEs and clinical outcomes: the first included Streptococcus spp. and was linked with adverse outcomes, and the second mostly included Lachnospiraceae spp. and was associated with favorable outcomes. Meta-analysis of microbial sequencing data using the same bioinformatic approach for four independent cohorts (Gopalakrishnan, V., et al. 2018; Matson, V., et al. 218; Frankel, A. E., et al. 2017; Peters, B. A., et al. 219), along with the cohort identified robust and reproducible beneficial and detrimental intestinal microbiome signatures in melanoma patients treated with anti-PD-1. Mapping of 16S rRNA gene sequencing microbiota data from the different cohorts to the American Gut Project cohort identified discrete taxonomic intestinal microbial communities that were associated with either response or resistance to anti-PD-1.

Collectively, the findings of this study provide a comprehensive evaluation of gut microbial sequencing data in checkpoint inhibitor treated melanoma and identify intestinal microbial signatures and potential associated mechanisms related to clinical outcome and occurrence of irAEs upon anti-PD-1/PD-L1, contributing new insights into the impact of intestinal microbiome composition upon outcome to anti-PD-1 or anti-PD-L1 therapy.

Accordingly, in some aspects, disclosed herein is a method of increasing effectiveness of an immune checkpoint inhibitor in a subject comprising administering to the subject the immune checkpoint inhibitor and increasing an amount of one or more bacteria in an intestine of the subject to a therapeutically effective amount, wherein the one or more bacteria are selected the bacteria listed in Table 4 and Table 8. In some examples, the one or more bacteria are selected from a Lachnospiraceae spp. In some examples, the one or more bacteria are selected from the group consisting of Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis.

In some aspects, disclosed herein is a method of increasing effectiveness of an immune checkpoint inhibitor in a subject comprising administering to the subject the immune checkpoint inhibitor and decreasing an amount of one or more bacteria in an intestine of the subject, wherein the one or more bacteria listed in Table 5 and Table 9. In some examples, the one or more bacteria are selected from a Streptococcus spp. In some embodiments, the one or more bacteria are selected from the group consisting of Bacteroides massiliensis, Bacteroides stercoris, Prevotella copri, and Bacteroides plebeius.

The methods disclosed herein increase a subject's responsiveness to an immune checkpoint inhibitor (e.g., a PD-1 inhibitor or a PD-L1 inhibitor) or reverse resistance to an immune checkpoint inhibitor (e.g., a PD-1 inhibitor or a PD-L1 inhibitor) in a subject, wherein the subject has a cancer (e.g., a melanoma). In some examples, the subject is resistant to immune checkpoint therapy. The methods disclosed herein are surprisingly effective to prevent and/or treat a cancer, metastasis, and/or recurrence.

The changes in levels of the group of bacteria disclosed herein (e.g., the bacteria listed in Tables 4, 5, 8 and 9) show effectiveness to predict a subject's responsiveness to an immune checkpoint inhibitor (e.g., a PD-1 inhibitor or a PD-L1 inhibitor). Accordingly, also disclosed herein is a method of predicting a subject's responsiveness to an immune checkpoint inhibitor comprising obtaining a stool sample from the subject and determining the presence or absence of one or more bacteria in the sample, wherein the one or more bacteria are selected from the group consisting of the bacteria listed in Tables 4, 5, 8, and 9.

### BRIEF DESCRIPTION OF THE DRAWINGS

FIG. 1(a-c). Compositional differences in the fecal microbiome of anti-PD-1 treated melanoma patients are associated with differential progression free survival (PFS). (a. and b.) Evaluation of association of fecal microbiome composition (Pittsburgh early sample cohort) and response to anti-PD-1 therapy were assessed using shotgun metagenomic (a) or 16S rRNA gene amplicon (b) sequencing. Objective radiographic response to therapy was evaluated by treating investigators using RECIST v1.1 every 3 months, and progression was defined based on radiographic or clinical progression at each treatment visit (every 3 or 4 weeks). Top left panels depict number of patients on follow up at each timepoint in relation to response status. Compositional differences of the initial microbiome using PERMANOVA 1/p of Bray-Curtis distance (y-axis, bottom left panels) were evaluated in progressors (Ps) and non-progressors (NPs) identified at each treatment visit, thus permitting inference of the optimal time (9-10 months) at which baseline intestinal microbiota composition reflected maximal separation between the clinical outcomes; the dots indicate PFS at all time points while the asterisk indicates RECIST v1.1 response (CR or PR) at 3 months. Right panels show t-distributed Uniform Manifold Approximation and Projection (t-UMAP) plots depicting fecal microbiota compositional differences between NPs and Ps at time of maximal difference from start of therapy (10 months). Open circles represent centroids with lines connecting them to corresponding samples from each group. p values were calculated using PERMANOVA. (c.) Metagenomic shotgun sequencing of fecal microbiota samples identifies differentially abundant taxa in Ps vs. NPs at 10 months from start of therapy. Heatmap shows differentially abundant taxa identified by metagenomic shotgun sequencing (FDR<0.2 and FC>2). Columns denote patients grouped by progression status and clustered within P/NP groups; rows denote bacterial taxa enriched (black font) or depleted (red font) in NPs versus Ps, clustered based on microbiota composition.

FIG. 2(a-b). Entire time-to-event progression data analysis by Cox regression method of baseline fecal microbiome composition identifies additional beneficial and detrimental taxa linked with response to anti-PD-1 immunotherapy. (a.) Volcano plot depicting bacteria identified by effect on PFS in the Pittsburgh Early Sample Cohort using Cox regression analysis in Evaluate Cutpoints software. Y axis is −Log10(FDR) while X axis depicts Log2[hazard ratio] of association with PFS. Representative taxa (red dots) are indicated. (b.) Cladogram visualization (beneficial taxa—blue; detrimental taxa—red) of bacterial taxa at different phylogenetic levels identified using approach described in (a).

FIG. 3(a-f) Relationship between microbiota composition, NLR and associated host variables in relation to clinical response. a. t-UMAP plots depicting fecal microbiota compositional differences between patient groups with high (≥3.82; orange) and low (<3.82; green) pre-treatment NLR. Optimal cutoff for NLR (3.82) was determined by time serial PERMANOVA similar to as shown in FIG. 1a. p value was calculated using PERMANOVA. (b.) Volcano plot of differentially expressed human host genes identified in stool specimens as described in Methods from NPs compared to Ps at 10 months in anti-PD-1 treated melanoma patients. (c.) Ingenuity pathway analysis of upstream regulators of differentially expressed host genes identified in (b). Predicted upstream regulators highlighted in yellow, while genes identified in (b) that were also predicted upstream regulators are highlighted in orange. (d.) GSEA analysis of predicted cell types based on host genes identified in (c) and differentially expressed in NP compared to P patients with significantly enriched (p<0.05) cell types depicted by red dots. (e.) Transkingdom network analysis of multi-omic data. Data for microbial taxa (circles), microbial genes (diamonds) and human genes (squares) were analyzed to identify highly differentially abundant elements between Ps and NPs in anti-PD-1 treated melanoma patients and integrated with phenotypes [brown hexagons: clinical outcome (PFS) and baseline variables (NLR)] to form a “transkingdom” network. Beneficial (blue) and detrimental (red) nodes (taxa, microbial- and host-genes) were defined as in FIGS. 1c and 1n Methods. Blue and orange edges indicate negative and positive correlations, respectively. Network interrogation revealed that microbes (taxa and genes) were strongly connected with human genes and phenotypes. (f.) Local and global properties of nodes in transkingdom network from (e). Degree (local property) (left panel) and LKT-Phenotype bipartite betweenness centrality (Bi-BC) (global property) (right panel) were calculated for the beneficial and detrimental taxa. p values were calculated by two-tail Mann Whitney test.

FIG. 4(a-f). Fecal microbial signatures are differentially associated with irAEs and PFS in PD-1-treated melanoma patients. (a.) tUMAP plot depicting compositional differences between melanoma patients who developed any irAEs and those who did not at any timepoint from start of anti-PD-1 therapy. p values were calculated using PERMANOVA. (b.) Heatmap of differentially abundant taxa (p<0.05 and FC>2) in patients with no irAEs vs. grade 1-4 irAEs. irAE severity was graded with CTCAE v5.0. Columns depict patients grouped by reported or non-reported irAE and clustered within corresponding groups based on gut microbiome composition. Rows depict bacterial taxa enriched (black) or depleted (red) in melanoma patients with irAEs (grade 1-4) and clustered based on gut microbiota composition. (c.) Kaplan-Meier plot of PFS probability based on irAE status. Numbers of patients at risk at each time point are shown. (d.) Percentages of PD-1 treated melanoma patients exhibiting specific types of irAE segregated by relative abundance of the 7 Streptococcus spp. identified in (b) (8 patients clustered in the high group). (e.) Kaplan-Meier plot of PFS probability based on relative abundance of Streptococcus spp. Numbers of patients at risk at each time point are shown. (f.) Proportion of melanoma patients treated with proton pump inhibitor among patients with high (left) vs. low (right) relative abundance of Streptococcus spp.

FIG. 5(a-d). Gut microbiome meta-analysis of five independent cohorts of melanoma patients treated with anti-PD-1 identifies organisms and microbial genes differentially enriched in Rs and NRs. (a.) After removing study-related batch effects using ComBat R package, resultant batch-corrected dataset was further analyzed using linear discriminant analysis effect size (LEfSe), and depicted using cladogram. Dot size is proportional to the abundance of the respective taxon. (b.) Fisher's method meta-analysis of differentially abundant shotgun-sequenced gut microbiome taxa (p<0.01) in Rs versus NRs from four publicly available melanoma patient cohorts, along with Pittsburgh Early Sample Cohort. Response to therapy in each of the published cohorts was determined as described in each study (Table 3). (c. and d.) Visualization of shared genes (blue) between significant taxa (orange) as determined by LEfSe from beneficial (c) and detrimental (d) sub-species from all 5 analyzed cohorts identified as described in Methods after removing study-related batch effects. The list of genes enriched in Rs or NRs is reported in FIG. 22.

FIG. 6(a-g). Mapping of combined 16S rRNA gene amplicon sequencing data from PD-1 treated melanoma patients to the American Gut Project dataset identifies beneficial and detrimental enteric microbiotypes. (a.) t-distributed stochastic neighbor embedding (t-SNE) plot of ˜7,000 stool samples from American Gut Project (AGP) dataset. Samples were clustered using PhenoGraph R package, and compositionally distinct clusters are displayed using different colors. (b.) Mapping of samples from four independent PD-1 treated melanoma patient cohorts (Chicago, Houston, New York, and Pittsburgh) with available 16S amplicon data onto AGP tSNE plot with each cohort depicted with a specific color (Chicago—green; Houston—blue; New York—yellow; Pittsburgh—red). (c.) Mapping of samples from (b) onto AGP tSNE plot with responders (blue) and non-responders (red) depicted separately. (d.) Distinct beneficial (blue) and detrimental (red) enteric microbiotypes were estimated by calculating odds ratios of response to non-response in each cluster as defined by PhenoGraph R package68. (e.) Manual segregation of beneficial and detrimental enteric superclusters from (d) onto AGP tSNE plot. (f) Heatmap depicts abundances of all taxa from the AGP project in beneficial and detrimental enteric superclusters. Dark red indicates most abundant, dark blue least abundant. (g.) Visualization using LEfSe cladogram of differentially abundant taxa in the four superclusters calculated on 16S amplicon data from the combined cohorts.

FIG. 7(a-h). Kaplan-Meier plots of PFS by abundance of select bacterial species in the Pittsburgh Early Sample Cohort. Optimal cutpoints of bacterial abundance were determined using Evaluate Cutpoints36. Different plots show the effect on PFS of abundance (high vs. low abundance) of the top 4 (a.-d.) most significantly increased (left panels) and decreased (right panels) individual bacterial species in non progressors at 10 months determined using Mann-Whitney U test (FIG. 1c). The number of people at risk in in either group (high vs. low abundance) is shown below each panel a.-h.

FIG. 8(a-d). The microbiota composition of NP patients in the Pittsburgh cohort whose stool samples were collected 4 to 41 months after initiation of the therapy was not predictive of late therapy failure but was enriched for similar bacterial taxa as observed in the initial microbiome of patients that did not progress at 10 months. (a.) Plot of time of stool sample acquisition from 31 patients whose samples were collected after more than 4 months from therapy initiation. (b.) P and NP groups identified at serial timepoints after the late stool collection (top panel) were used to calculate compositional differences of the late collected fecal microbiome using PERMANOVA (bottom panel). Fecal microbiota composition was determined using metagenomic sequencing. Progression during continued therapy was evaluated using RECIST v1.1 every 3 months. Number of patients on follow-up at each timepoint in relation to response status is shown in top panel. (c.) t-UMAP plot (left) depicts fecal microbiota compositional differences between early collected patients that progressed within 10 months (red) and late collected long-term responders (green). Heatmap (right) shows differentially abundant taxa (p<0.05 and FC>2) between these two cohorts. Columns denote patients grouped by each cohort before clustering; rows denote bacterial taxa enriched (black font) or depleted (red font) in early sampled progressor versus late sampled long-term non-progressor clustered based on microbiota composition. p values were calculated using PERMANOVA and Mann-Whitney U test. (d.) t-UMAP plot (left) depicts fecal microbiota compositional differences between early collected patients that did not progress in the first 10 months after initiation of therapy (blue) and late collected long-term non-progressor (green). Heatmap (right) shows differentially abundant taxa (p<0.05 and FC>2) between these two cohorts. Columns denote patients grouped by each cohort before clustering; rows denote bacterial taxa enriched (black) or depleted (red) in early sampled progressor versus late sampled long-term non-progressor clustered based on microbiota composition. p values were calculated using PERMANOVA and Mann-Whitney U test.

FIG. 9. Differential abundance analysis reveals relationship of baseline gut microbial taxa with high vs. low NLR in Pittsburgh Early Sample cohort. Heatmap of differentially abundant taxa (p<0.05 and FC>2) in high pretreatment NLR (orange) and low NLR (green) patients, using an optimal cutoff (3.82) determined by time serial PERMANOVA similar as shown in FIG. 1a. Columns denote patients grouped by NLR status and clustered within each group; rows denote bacterial taxa that were enriched (red) in patients with high NLR clustered based on microbiota composition; no bacterial taxa significantly enriched in the low NLR patients was identified. Bar plot to the left of the heatmap indicates extent of association between corresponding taxa and PFS probability (scaled HR) with p values 0.1 displayed within cells.

FIG. 10(a-d). Gut microbial gene differences discriminate between NPs and Ps during anti-PD-1 therapy in the Pittsburgh Early Sample cohort. (a.) t-UMAP plot depicting genetic differences of gut microbiomes between NPs (blue) and Ps (red) at time of maximal difference from start of therapy (10 months). Filled circles represent centroids with lines connecting them to corresponding samples from each group. p value was calculated using PERMANOVA. (b.) Metagenomic shotgun sequencing of fecal microbiota samples identifies differentially abundant genes in Ps vs. NPs at 10 months from start of therapy. Heatmap shows differentially abundant genes identified by metagenomic shotgun sequencing (FDR<0.2 and FC>1.5). Columns denote patients grouped by progression status and clustered within P/NP groups; rows denote bacterial genes significantly upregulated (red) or downregulated (blue) in Ps versus NPs. (c. and d.) Select genes involved in representative microbial processes [lipopolysaccharide (LPS) and iron metabolism] in Ps (c) and NPs (d) are shown.

FIG. 11. Metagenomic sequencing identifies distinct taxa associated with various irAEs in PD-1-treated melanoma patients in Pittsburgh early cohort. Heatmap depicts metagenomic compositional differences between patients with distinct irAEs as compared to patients with other irAE using scaled fold differences (high—red; low—blue) in abundances of specific bacteria. Values in individual cells represent unadjusted p values calculated using Mann-Whitney U test with p values≤0.1 displayed within cells. Bar plot to the left of the heatmap indicates extent of association between corresponding taxa and PFS probability (scaled HR) with p values 0.1 displayed within cells.

FIG. 12(a-c). Reanalysis of four previously published individual cohorts using the same bioinformatic pipeline. (a.) Analysis of a-diversity from five PD-1-treated melanoma patient cohorts (n=185), including the Pittsburgh early sample cohort (n=63), using either shotgun metagenomic (5 cohorts, red) or 16S rRNA gene amplicon (4 cohorts, black) sequencing. Details of each individual cohort are summarized in Table 3. Forest plots depict a-diversity-based association tests including inverse Simpson), Shannon and observed operational taxonomic units. Within each fixed-effect plot, names of each cohort are shown on a separate line, while the log odds ratio of u-diversity (squares, size proportional to sample size used in meta-analysis) and associated 95% confidence intervals (bars) are shown along with the dotted vertical line of no effect. To control for unobserved heterogeneity, we separately evaluated a-diversity using a random effects model on both pooled shotgun and 16S sequencing data from the 5 cohorts and performed 12 test for heterogeneity as shown. (b.) t-UMAP plot before (left) and after (right) correction for study-related batch effect using ComBat R package for all cohorts together including Pittsburgh cohort p values were calculated using PERMANOVA. (c.) t-UMAP plot of batch-corrected pooled metagenomic sequencing data from five separate cohorts of melanoma patients treated with anti-PD-1 therapy depicting fecal microbiota compositional differences calculated using PERMANOVA between Rs and NRs. p value was calculated using PERMANOVA.

FIG. 13. Differential abundance analysis of batch-corrected metagenomic data from five melanoma patient cohorts. Heatmap of differentially abundant gut microbiome taxa (p<0.05, FC>2) evaluated with shotgun sequencing in five melanoma patient cohorts, including Pittsburgh Early Sample cohort. Study-related batch effect was removed using ComBat R package. Response to therapy in the published cohorts was determined as described in each study (Table 3). Response to therapy in the Pittsburgh Early Sample cohort was defined as non-progression at 10 months after initiation of treatment. Columns represent patients grouped by clinical response and clustered within R/NR groups; rows depict bacterial taxa enriched (black font) or depleted (red font) in Rs versus NRs clustered based on gut microbiota composition. p values were calculated using Mann-Whitney U test.

FIG. 14(a-b). Meta-analysis of all cohorts using random effects model identifies organisms differentially enriched in melanoma patients treated with anti-PD-1 therapy in separate cohorts by response status. (a.) Random effect model meta-analysis of differentially abundant bacteria between Rs and NRs from five cohorts (n=185) including Pittsburgh Early Sample cohort (n=63) using shotgun metagenomic sequencing. All significant bacterial taxa enriched in Rs and NRs are shown. (b.) Forest plots depict association of representative bacterial species with response to anti-PD-1 therapy. Within each plot, names of various cohorts are shown on separate lines, while Hedge's G (squares, size proportional to sample size) and associated 95% confidence intervals (bars) are shown along with the dotted vertical line of no effect. To control for unobserved heterogeneity, we separately evaluated Hedge's G using random effect model on metagenomic data and performed 12 test for heterogeneity as shown.

FIG. 15. Expression level of selected taxa in different AGP enteric microbiotypes. tSNE plot depicting AGP dataset with visualization of abundances of select taxa (blue—low versus red—high).

FIG. 16(a-d). Geographic differences determine sampling variability between cohorts. (a.) tSNE plot depicting mapping of individual melanoma patient cohorts to AGP dataset revealed distinct compositional differences between them. (b. and c.) Heatmaps represent scaled abundances of each enteric microbiotypes across 28 states from which AGP data was available on the left with the four states from which the anti-PD-1 treated melanoma cohorts originated separately on the right. (b.) Shows heatmap scaled only by number of samples from state (to reflect the local abundance of microbiotypes). (c.) shows heatmap scaled by both number of samples per state and by number of samples per microbiotype (to reflect distribution of different microbiotypes across the USA) (d.) Geographic representation in the United States of four representative enteric microbiotypes with most uneven distribution between the four states (right panel c.).

FIG. 17(a-c). Distribution of beneficial and detrimental taxa is similar in anti-PD-1 alone and pembrolizumab+pegIFN treated patients. (a and b). t-UMAP plot depicting differences of gut microbial taxa between NPs and Ps at time of maximal difference from start of therapy (10 months) in metastatic melanoma patients treated with pembrolizumab/pegIFN (a) and anti-PD-1 alone (b). Empty circles represent centroids with lines connecting them to corresponding samples from each group. P values were calculated using PERMANOVA. c. Heatmap of hierarchically clustered differentially abundant taxa (p<0.05, FC>2) from anti-PD-1 alone cohort. Each column represents an independent melanoma patient, while each row represents differentially abundant bacteria.

FIG. 18. Kaplan-Meier plots of PFS by bacterial abundance (Pittsburgh Early Sample Cohort). The different panels show the effect of representative bacterial species selected among the most significant ones identified by beneficial or detrimental effect on PFS in the Pittsburgh Early Sample Cohort using Cox regression analysis in Evaluate Cutpoints software.

FIG. 19. Complete list of taxa associated with various irAEs in melanoma patients treated with anti-PD-1 immunotherapy in Pittsburgh cohort. Heatmap depicts metagenomic compositional differences between patients with distinct irAEs as compared to patients with other irAE using scaled fold differences (high—red; low—blue) in abundances of specific bacteria between patients with distinct irAEs as compared to patients with other irAEs. Values in individual cells represent unadjusted p values calculated using Mann-Whitney U test with p values≤0.1 displayed within cells.

FIG. 20(a-b). PPI exposure is associated with differential abundance of select taxa and shorter PFS in Pittsburgh cohort. (a.) Effect of PPI exposure at the time of stool collection upon PF S using Cox proportional hazard model in samples from Pittsburgh is shown. The number of people at risk in in either group is shown below each panel. (b.) Heatmap shows hierarchically clustered differentially abundant taxa (p<0.1, FC>2) between patients with PPI exposure as compared to patients without PPI exposure. Each column represents an independent melanoma patient, while each row represents differentially abundant bacteria.

FIG. 21(a-d). Heatmaps of differentially abundant taxa analyzed using the same bioinformatic pipeline in four previously published cohorts of melanoma patients treated with anti-PD-1 immunotherapy. (a-d). Each heatmap depicts differentially abundant taxa identified by metagenomic shotgun sequencing (p<0.05, FC>2) from four independent melanoma cohorts (a—Houston; b—Chicago; c—New York; d—Dallas). Columns denote patients grouped by response status. Response to therapy in the published cohorts was determined as described in each study (Table S). Rows denote bacterial taxa enriched (black font) or depleted (red font) in Rs versus NRs clustered based on microbiota composition.

FIG. 22(a-b). Microbial genes differentially enriched in Rs and NRs by fecal microbiome meta-analysis of five independent cohorts of anti-PD-1 treated melanoma patients. After removing study-related batch effects using ComBat R package, resultant batch-corrected dataset was further analyzed using linear discriminant analysis effect size (LEfSe).

FIG. 23(a-d). Compositional differences in the gut microbiome of anti-PD-1 treated melanoma patients from Houston are associated with differential objective response and PFS. (a.) Evaluation of effects of initial (pre-treatment up to 3 months on treatment) fecal microbiome composition upon investigator-assessed response to anti-PD-1 therapy was evaluated using shotgun sequencing. Top panel depicts number of patients on follow up at each timepoint in relation to response status. Compositional differences of the initial microbiome using PERMANOVA 1/p (y-axis, bottom panel) were evaluated in Ps and NPs identified at each treatment visit, thus permitting inference of the optimal time to predict impact of microbiome composition upon response (13 months). (b.) t-UMAP plot depicting fecal microbiota compositional differences between NPs and Ps at time of maximal difference from start of therapy (13 months). Empty circles represent centroids with lines connecting them to corresponding samples from each group. p values were calculated using PERMANOVA. (c.) Metagenomic shotgun sequencing of fecal microbiota samples identifies differentially abundant taxa in Ps vs. NPs at 13 months from start of therapy. Heatmap shows differentially abundant taxa identified by metagenomic shotgun sequencing (p<0.05 and FC>2). Columns denote patients grouped by progression status and clustered within NP/P groups; rows denote bacterial taxa enriched (black font) or depleted (red font) in NPs versus Ps clustered based on microbiota composition. (d.) Venn diagram showing overlap of taxa identified the Houston and Pittsburgh cohorts by effect on PFS using Cox regression analysis in Evaluate Cutpoints software36 as being significantly (p<0.05) associated with favorable (left panel, enriched in NPs) vs. unfavorable (right panel, enriched in Ps) response to anti-PD-1 therapy.

FIG. 24(a-b). Performance of different machine learning algorithms on baseline metagenomic data to predict progression status of anti-PD-1 treated patients. a and b. To estimate the accuracy of baseline metagenomic data from any given dataset (rows) in predicting progression status, we utilized two separate validation techniques: 70:30 train test splitting (a), and training models on all but one dataset which is used for testing (b), as described in Methods. Algorithms from left to right: random forest model (rf), support vector machine model (SVM), generalized linear models (glm), eXtreme Gradient Boosting with Dropouts meet Multiple Additive Regression Trees model (xgbDART) 108. Machine learning models were either trained on a given dataset and tested to predict response status in the larger dataset that excluded values from the training set (a, train test splitting); or conversely, trained on the entire dataset that excluded a given dataset, and subsequently tested in the given dataset (b, leave-one-out). Accuracy of each model in predicting outcome was summarized using several metrics: accuracy (percentage of correctly classified instances out of all instances), area under the receiver operative curve (AUC), kappa (normalized accuracy). AUC values are shown within each cell, color coded by heatmap on right. p values were calculated using binomial test. (* P<0.05 ** P<0.01).

FIG. 25. Correlogram of taxa abundance to each other using metagenomic data from 5 melanoma cohorts. The top 330 most variant taxa across samples are shown. Bar on right represents taxa significantly associated (blue—beneficial; red—detrimental) with response or non-response to anti-PD-1 therapy, while bars on the bottom represents phylum and Gram stain characteristics of each taxa.

FIG. 26(a-b). Enteric microbiotypes identified in the AGP database and their distribution within the 4 cohorts of PD-1-treated melanoma patients. (a.) The heatmap shows the distribution of differentially abundant taxa in different clusters/microbiotypes from AGP fecal microbiota dataset identified using PhenoGraph R package68. (b.) The nested pie charts show the relative distribution of microbiotypes and beneficial or detrimental superclusters within four cohorts of PD-1-treated melanoma patients.

### DETAILED DESCRIPTION OF THE INVENTION

Described herein is the surprising finding that there are beneficial and detrimental signatures in baseline fecal microbiota composition in relation to immune checkpoint inhibitor therapies. Accordingly, provided herein are methods of predicting a subject's responsiveness to an immune checkpoint inhibitor comprising obtaining a stool sample from the subject and determining the presence or absence of one or more bacteria in the sample, wherein the one or more bacteria are selected from the groups consisting of the beneficial and/or detrimental groups of bacteria. Also provided are methods for increasing a subject's responsiveness to an immune checkpoint inhibitor and methods of treating a cancer comprising increasing a level or amount of one or more beneficial bacteria and/or decreasing a level or amount of a detrimental bacteria.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is an anti-PD-1 refractory cancer. In some embodiments, the cancer is a melanoma. In some embodiments, the cancer is an anti-PD-1 refractory melanoma.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.” In some embodiments, the control described herein is a negative control wherein the control subject or control population receives an immune checkpoint inhibitor, but does receive a treatment according to the present invention.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENTM (ICI, Inc.; Bridgewater, New Jersey), polyethylene glycol (PEG), and PLURONICSTM (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “increasing effectiveness of an immune checkpoint inhibitor” can refer to any measure of increased effectiveness including an improved treatment of a cancer as compared to a control. In some embodiments, increased effectiveness of an immune checkpoint inhibitor is demonstrated by a decrease in tumor growth, a decrease in tumor volume or size, a decrease in tumor number, and/or a decrease in cancer metastasis, all as compared to a control subject or control population. In some embodiments, increased effectiveness of an immune checkpoint inhibitor is demonstrated by a decrease in an immune related adverse event. Immune related adverse events (irAEs) include, but are not limited to, pneumonitis, arthritis, hepatitis, adverse cutaneous side effects (e.g., skin and subcutaneous disorders including rash, pemphigoid, and pruritus), adrenal insufficiency, muscle inflammation, thyroid abnormality (including hyperthyroidism, hypothyroidism, thyroiditis), colitis, nephritis, and adverse neurological side effects (e.g., Guillain-Barre syndrome, encephalitis, and myasthenic syndrome).

As used herein “immune checkpoint inhibitor” or “checkpoint inhibitor” refers to a molecule that completely or partially reduces, inhibits, interferes with or modulates one or more checkpoint proteins. Checkpoint proteins include, but are not limited to, PD-1, PD-L1 and CTLA-4. Control samples (untreated with inhibitors) are assigned a relative activity value of 100%. Inhibition of a described target protein is achieved when the activity value relative to the control is about 80%, 50%, 25%, 10%, 5%, or 1% or less.

As used herein, the term “PD-1 inhibitor” refers to a composition that reduces or inhibits the interaction between PD-1 and PD-L1. In some embodiments, the PD-1 inhibitor binds to PD-1 and reduces or inhibits the interaction between the bound PD-1 and PD-L1. In some embodiments, the PD-1 inhibitor is a monoclonal antibody that is specific for PD-1 and that reduces or inhibits the interaction between the bound PD-1 and PD-L1. Non-limiting examples of PD-1 inhibitors are pembrolizumab, nivolumab, and cemiplimab. In some embodiments, the pembrolizumab is KEYTRUDA or a bioequivalent. In some embodiments, the pembrolizumab is that described in U.S. Pat. Nos. 8,952,136, 8,354,509, and/or 8,900,587, all of which are incorporated by reference in their entireties. In some embodiments, the pembrolizumab has the Unique Ingredient Identifier (UNII) of the U.S. Food and Drug Administration of DPT003T46P. In some embodiments, the nivolumab is OPDIVO or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. Nos. 7,595,048, 8,738,474, 9,073,994, 9,067,999, 8,008,449, and/or 8,779,105, all of which are incorporated by reference in their entireties. In some embodiments, the cemiplimab is LIBTAYO or a bioequivalent. In some embodiments, the cemiplimab has the Unique Ingredient Identifier (UNII) of the U.S. Food and Drug Administration of 6QVL057INT. In some embodiments, the cemiplimab is that described in U.S. patent Ser. No. 10/844,137, which is incorporated by reference in its entirety.

The term “PD-L1 inhibitor” refers to refers to a composition that reduces or inhibits the interaction between PD-L1 and PD-1. In some embodiments, the PD-L1 inhibitor binds to PDL-1 and reduces or inhibits the interaction between the bound PD-L1 and PD-1. In some embodiments, the PD-L1 inhibitor is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ or a bioequivalent. In some embodiments, the atezolizumab has the Unique Ingredient Identifier (UNII) of the U.S. Food and Drug Administration of 52CMIOWC3Y. In some embodiments, the atezolizumab is that described in U.S. Pat. No. 8,217,149, which is incorporated by reference in its entirety. In some embodiments, the avelumab is BAVENCIO or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI or a bioequivalent. In some embodiments, the durvalumab has the Unique Ingredient Identifier (UNII) of the U.S. Food and Drug Administration of 28×28×90 KV. In some embodiments, the durvalumab is that described in U.S. Pat. No. 8,779,108, which is incorporated by reference in its entirety.

The term “CTLA-4 inhibitor” refers to refers to a composition that reduces or inhibits the interaction between CTLA-4 and CD80/CD86. In some embodiments, the CTLA-4 inhibitor binds to CTLA-4 and reduces or inhibits the interaction between the bound CTLA-4 and CD80/CD86. In some embodiments, the CTLA-4 inhibitor is a monoclonal antibody that is specific for CTLA-4 and that reduces or inhibits the interaction between the bound CTLA-4 and CD80/CD86. Non-limiting examples of CTLA-4 inhibitors are ipilimumab and tremelimumab. In some embodiments, the ipilimumab is YERVOY or a bioequivalent. In some embodiments, the ipilimumab has the Unique Ingredient Identifier (UNII) of the U.S. Food and Drug Administration of 6T8C155666. In some embodiments, the ipilimumab is that described in U.S. Pat. Nos. 7,605,238 and 6,984,720, which are incorporated by references in their entireties. In some embodiments, tremelimumab has the Unique Ingredient Identifier (UNII) of the U.S. Food and Drug Administration of QEN1X95CIX. In some embodiments, the tremelimumab is that described in U.S. Pat. No. 6,682,736, which is incorporated by reference in its entirety.

The term “reduced”, “reduce”, “suppress”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a disease (e.g., a cancer).

“Therapeutically effective amount” or “therapeutically effective dose” of a composition refers to an amount that is effective to achieve a desired therapeutic result. In some embodiments, a desired therapeutic result is a reduction of tumor size. In some embodiments, a desired therapeutic result is a reduction of cancer metastasis. In some embodiments, a desired therapeutic result is a reduction in amount or size of a skin cancer. In some embodiments, a desired therapeutic result is a reduction in amount or size of a melanoma. In some embodiments, a desired therapeutic result is the prevention of cancer relapse. In some embodiments, a desired therapeutic result is a reduction in an immune related adverse event. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as control of tumor growth. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Methods

Provided herein are methods of increasing effectiveness of an immune checkpoint inhibitor in a subject comprising administering to the subject the immune checkpoint inhibitor and increasing an amount of one or more bacteria in an intestine of the subject to a therapeutically effective amount, wherein the one or more bacteria are selected from the group consisting of bacteria listed in Table 4 and Table 8 (e.g., Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Enterocloster clostridioformis, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Enterocloster lavalensis, Faecalibacterium prausnitzii, Eubacterium limosum, an AnaerotruncusUnclassified, Duodenibacillus massiliensis, Faecalimonas umbilicata, Absiella dolichum, Prevotella oryzae, Prevotella lascolaii, Prevotella, Prevotella koreensis, Oxalobacter formigenes, Prevotella stercorea, a Prevotella Unclassified, Prevotella copri, Prevotella buccae, Bacteroides caccae, Lactobacillus paracasei, Acinetobacter baumannii, Acidaminococcus fermentans, Lactobacillus rhamnosus, a Bacillus Unclassified, and a Faecalibacterium Unclassified). In certain aspects, the one or more bacteria are selected from the group consisting of Blautia coccoides, Lachnospiraceae, Ruminococcus torques, Absiella dolichum, Enterocloster bolteae, Lachnospiraceae bacterium, Enterorhabdus caecimuris, Collinsella intestinalis, Actinomyces oris, Enterocloster clostridioformis, Bacteroides eggerthii, Blautia hansenii, Dorea longicatena, Coprococcus comes, Clostridiaceae bacterium, Bacteroides thetaiotaomicron, Anaerostipes caccae, Adlercreutzia equolifaciens, Lactobacillus crispatus, Enterocloster aldensis, Mogibacterium diversum, Parabacteroides johnsonii, Eubacterium rectale, Schaalia odontolytica, Ruminococcus gnavus, Akkermansiaceae, Eggerthellaceae, Sellimonas intestinalis, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, Anaeromassilibacillus Unclassified, Tyzzerella nexilis, Blautia schinkii, Enterocloster lavalensis, Faecalibacterium prausnitzii, Eubacterium limosum, Anaerotruncus Unclassified, Duodenibacillus massiliensis, Faecalimonas umbilicata, Pseudomonas aeruginosa, Massilistercora timonensis, Eisenbergiella massiliensis, Eubacterium, Hungatella hathewayi, Butyrivibrio crossotus, Lactobacillus rhamnosus, Parabacteroides Unclassified, Clostridium spiroforme, Bariatricus massiliensis, Erysipelotrichaceae bacterium, Holdemanella biformis, Butyrivibrio Unclassified, Blautia Unclassified, Blautia hydrogenotrophica, Eubacterium ruminantium, Clostridioides difficile, Blautia, Erysipelotrichaceae, Gordonibacter urolithinfaciens, Frisingicoccus caecimuris, Clostridium scindens, Bifidobacterium pseudocatenulatum, Eubacterium Unclassified, Ruminococcus Unclassified, Coprobacillus, Gemmiger formicilis, Sharpea azabuensis, Clostridium symbiosum, Eubacteriaceae, Parabacteroides distasonis, Coprobacillus Unclassified, Clostridium Unclassified, Muribaculaceae bacterium, Clostridium sporosphaeroides, Lachnoclostridium phocaeense, Anaerofustis stercorihominis, Phascolarctobacterium succinatutens, Blautia producta, Bacillales, Clostridium methylpentosum, Clostridium leptum, Intestinimonas butyriciproducens, Anaerotruncus colihominis, Parabacteroides, Lachnospira multipara, Coprococcus catus, Ruminococcaceae, Coprococcus Unclassified, Butyricicoccus, Neglecta timonensis, Bifidobacterium longum, Clostridium phoceensis, Clostridium innocuum, Phascolarctobacterium faecium, Dorea Unclassified, Blautia obeum, Holdemania filiformis, Clostridium disporicum, Firmicutes, Bifidobacterium bifidum, Bifidobacterium adolescentis, Ruminococcaceae bacterium, Firmicutes bacterium, Phocea massiliensis, Alistipes onderdonkii, Clostridiaceae, Lactococcus lactis, Mogibacterium pumilum, Barnesiella intestinihominis, Bacteroides xylanisolvens, Blautia wexlerae, Lactobacillus vaginalis, Clostridium viride, Anaerostipes hadrus, Marvinbryantia formatexigens, Bacteroides uniformis, Oscillospiraceae, Streptococcus thermophilus, Coriobacteriaceae, Terrisporobacter glycolicus, and Fusicatenibacter saccharivorans. In certain aspects, the one or more bacteria are selected from a Lachnospiraceae spp. In certain aspects, the one or more bacteria are selected from the group consisting of Lachnospiraceae spp., Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis. In some embodiments, an amount of a Lachnospiraceae spp. is increased in the subject. In some embodiments, an amount of Ruminococcus (Mediterraneibacter) torques is increased in the subject. In some embodiments, an amount of Ruminococcus (Mediterraneibacter) gnavus is increased in the subject. In some embodiments, an amount of Blautia wexlerae is increased in the subject. In some embodiments, an amount of Blautia hansenii gnavus is increased in the subject. In some embodiments, an amount of Eubacterium rectale is increased in the subject. In some embodiments, an amount of Actinomyces bouchesdurhonensis is increased in the subject. In some embodiments, the one or more bacteria selected from the group consisting of bacteria listed in Table 4 and Table 8 are increased concurrently with the administration of the immune checkpoint inhibitor. In other embodiments, the one or more bacteria selected from the group consisting of bacteria listed in Table 4 and Table 8 are increased in a subject after the subject has received an immune checkpoint inhibitor and has been determined to be unresponsive to the inhibitor. In some embodiments, the one or more bacteria selected from the group consisting of bacteria listed in Table 4 and Table 8, or the group consisting of a Lachnospiraceae spp, Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis, is administered to the subject.

In some embodiments, the one or more bacteria are increased to a therapeutically effective amount through administration of the one or more bacteria to the subject. The administration may be of any type known to those of skill in the art. In some embodiments, the administration is in the form of an oral dosage. In some embodiments, the oral dosage form is a capsule or pill. The administration of the one or more bacteria can occur prior to the administration of the immune checkpoint inhibitor or after the administration of the immune checkpoint inhibitor. Appropriate dosages can be determined by one of ordinary skill in the art and include exemplary dosage amounts for a mammal of from about 0.5 to about 200 mg/kg of body weight of active composition per day, which can be administered in a single dose or in the form of individual divided doses, such as from 1 to 4 times per day. Alternatively, the dosage amount can be from about 0.5 to about 150 mg/kg of body weight of active composition per day, about 0.5 to 100 mg/kg of body weight of active compound per day, about 0.5 to about 75 mg/kg of body weight of active compound per day, about 0.5 to about 50 mg/kg of body weight of active composition per day, about 0.5 to about 25 mg/kg of body weight of active composition per day, about 1 to about 20 mg/kg of body weight of active composition per day, about 1 to about 10 mg/kg of body weight of active composition per day, about 20 mg/kg of body weight of active composition per day, about 10 mg/kg of body weight of active composition per day, or about 5 mg/kg of body weight of active composition per day. In some embodiments, the oral dosage form comprises about 1.0×10⁸ CFUs to about 5.0×10¹⁰ CFUs, about 1.0×10⁸ CFUs to about 5.0×10⁸ CFUs, about 1.0×10⁸ CFUs to about 1.0×10⁹ CFUs, about 1.0×10⁹ CFUs to about 5.0×10⁹ CFUs, about 1.0×10⁹ CFUs to about 1.0×10¹⁰ CFUs, about 1.0×10¹⁰ CFUs to about 5.0×10¹⁰ CFUs, about 1.0×10⁸ CFUs to about 1.0×10⁹ CFUs, about 5.0×10⁸ CFUs to about 5.0×10⁹ CFUs of one or more of Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Enterocloster clostridioformis, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Enterocloster lavalensis, Faecalibacterium prausnitzii, Eubacterium limosum, an Anaerotruncus Unclassified, Duodenibacillus massiliensis, Faecalimonas umbilicata, Absiella dolichum, Prevotella oryzae, Prevotella lascolaii, Prevotella, Prevotella koreensis, Oxalobacter formigenes, Prevotella stercorea, a Prevotella Unclassified, Prevotella copri, Prevotella buccae, Bacteroides caccae, Lactobacillus paracasei, Acinetobacter baumannii, Acidaminococcus fermentans, Lactobacillus rhamnosus, a Bacillus Unclassified, and a Faecalibacterium Unclassified.

In some embodiments, the administration of the one or more bacteria occurs concurrently with the administration of the immune checkpoint inhibitor. In some embodiments, the administration of the one or more bacteria occurs within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6,7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more after the immune checkpoint inhibitor is administered to the subject. In some embodiments, the administration of the one or more bacteria occurs within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more before the immune checkpoint inhibitor is administered to the subject.

As described above, the immune checkpoint inhibitor can be any of a PD-1, PDL-1 or CTLA-4 inhibitor. In some embodiments, the immune checkpoint inhibitor comprises a PD-1 inhibitor (e.g., pembrolizumab, nivolumab, cemiplimab, dostarlimab, spartalizumab, JTX-4014, camrelizumab, sintilimab, tislelizumab, toripalimab, INCMGA00012 (MGA012), AMP-224, or AMP-514 (MEDI0680)), a PD-L1 inhibitor (e.g., atezolizumab, avelumab, durvalumab, CK-301, or BMS-986189), or a CTLA-4 inhibitor (e.g., ipilimumab or Tremelimumab). In some embodiments, the immune checkpoint inhibitor is an anti-PD-1 composition. In some embodiments, the immune checkpoint inhibitor is an anti-PD-1 antibody. As used herein, the term “PD-1 inhibitor” refers to a composition that binds to PD-1 and reduces or inhibits the interaction between PD-1 and PD-L1. In some embodiments, the PD-1 inhibitor binds to PD-1 and reduces or inhibits the interaction between the bound PD-1 and PD-L1. In some embodiments, the PD-1 inhibitor is a monoclonal antibody that is specific for PD-1 and that reduces or inhibits the interaction between the bound PD-1 and PD-L1. Non-limiting examples of PD-1 inhibitors are pembrolizumab, nivolumab, and cemiplimab. In some embodiments, the pembrolizumab is KEYTRUDA or a bioequivalent. In some embodiments, the pembrolizumab is that described in U.S. Pat. Nos. 8,952,136, 8,354,509, or U.S. Pat. No. 8,900,587, all of which are incorporated by reference in their entireties. In some embodiments, the pembrolizumab has the Unique Ingredient Identifier (UNII) of the U.S. Food and Drug Administration of DPT003T46P. In some embodiments, the nivolumab is OPDIVO or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. Nos. 7,595,048, 8,738,474, 9,073,994, 9,067,999, 8,008,449, or U.S. Pat. No. 8,779,105, all of which are incorporated by reference in their entireties. In some embodiments, the cemiplimab is LIBTAYO or a bioequivalent. In some embodiments, the cemiplimab has the Unique Ingredient Identifier (UNII) of the U.S. Food and Drug Administration of 6QVL057INT. In some embodiments, the cemiplimab is that described in U.S. patent Ser. No. 10/844,137, which is incorporated by reference in its entirety.

In other or further embodiments, provided are methods of increasing effectiveness of an immune checkpoint inhibitor in a subject comprising administering to the subject the immune checkpoint inhibitor and decreasing an amount of one or more bacteria in an intestine of the subject, wherein the one or more bacteria are selected from the group consisting of bacteria listed in Table 5 and Table 9 (e.g., Prevotella koreensis, Prevotella oryzae, Intestinimonas butyriciproducens, Bacteroidales bacterium, Alistipes senegalensis, a Subdoligranulum Unclassified, Sporobacter termitidis, a Streptomyces Unclassified, Pseudoflavonifractor, Clostridium perfringens, Veillonella parvula, Oscillibacter ruminantium, a Prevotella Unclassified, Haemophilus, Lactobacillus fermentum, Proteobacteria, Streptococcus mutans, Ruminococcus flavefaciens, Prevotella buccae, Eubacterium pyruvativorans, Oscillibacter valericigenes, Bacillus, Cuneatibacter caecimuris, Acinetobacter baumannii, Actinobacteria, Porphyromonas uenonis, Alistipes dispar, Christensenella minuta, Veillonella, Streptococcus vestibularis, Fournierella massiliensis, Eubacterium siraeum, Agathobaculum desmolans, Oxalobacter formigenes, Bacteroidales, Victivallales bacterium, Alistipes finegoldii, Bacteroides ilei, Intestinimonas massiliensis, Lachnospira eligens, a Bacteriophage Unclassified, Odoribacter splanchnicus, a Bacillus Unclassified, Angelakisella massiliensis, Haemophilus parainfluenzae, Ruminococcus bromii, a Flintibacter Unclassified, Sutterellaceae, Alistipes shahii, Bacteroides caccae, Odoribacter laneus, Alistipes communis, Acidaminococcus, Rikenellaceae bacterium, Klebsiella, Bacteroides faecis, Coprobacter fastidiosus, Collinsella, a Paenibacillus Unclassified, a Pseudoflavonifractor Unclassified, Streptococcus, a Faecalibacterium Unclassified, Ruminococcus albus, Eukaryota, Alistipes timonensis, an Oscillibacter Unclassified, Lactobacillus salivarius, Intestinibacillus massiliensis, a Haemophilus Unclassified, Acidaminococcus fermentans, Streptococcus salivarius, a Muribaculum Unclassified, Clostridiales bacterium, Lactobacillus, Flavonifractor plautii, Faecalibacterium, Bacteroides massiliensis, Lactobacillus paracasei, Ruminococcus lactaris, Subdoligranulum, Anaerotruncus massiliensis, Ruminococcus champanellensis, Enterobacter hormaechei, Clostridium, Cyclospora cayetanensis, Anaeromassilibacillus senegalensis, Bacteroides ovatus, Butyricimonas virosa, Ruthenibacterium lactatiformans, Bacteroides finegoldii, Alistipes putredinis, a Butyricicoccus Unclassified, Bacteroides cellulosilyticus, Bacteroides coprocola, Intestinimonas timonensis, Victivallis vadensis, Sutterella wadsworthensis, Clostridium botulinum, Coprococcus eutactus, Anaerotignum lactatifermentans, a Duncaniella Unclassified, Escherichia, an Acidaminococcus Unclassified, Dysosmobacter welbionis, Butyricimonas, Bacteroides intestinalis, Bilophila wadsworthia, Desulfovibrio fairfieldensis, Lactobacillus gasseri, Agathobaculum butyriciproducens, Klebsiella pneumoniae, Faecalitalea cylindroides, Porphyromonas asaccharolytica, Harryflintia acetispora, Butyricicoccus pullicaecorum, Lactobacillus paragasseri, Alistipes indistinctus, an Akkermansia Unclassified, Cryptobacterium curtum, Bacteroides vulgatus, Oscillibacter, a Prevotella Unclassified, Prevotella, Butyricimonas virosa, Subdoligranulum, Bacteroides massiliensis, Prevotella copri, Enterocloster clostridioformis, Prevotella stercorea, Prevotella lascolaii, Desulfovibrio fairfieldensis, Anaerotruncus colihominis, Streptococcus parasanguinis, Eubacterium maltosivorans, Bacteroides plebeius, a Megasphaera Unclassified, a Desulfovibrio Unclassified, Escherichia coli, Desulfovibrio desulfuricans, Enterobacteriaceae, Parabacteroides merdae, Akkermansia muciniphila, Paraprevotella clara, Enterocloster lavalensis, and Dialister invisus). In certain aspects, the one or more decreased bacteria are selected from Prevotella oryzae, Haemophilus, Lactobacillus fermentum, Proteobacteria, Streptococcus mutans, Prevotella buccae Eubacterium pyruvativorans, Acinetobacter baumannii, Porphyromonas uenonis, Alistipes dispar Veillonella, Streptococcus vestibularis, Eubacterium siraeum, Akkermansia muciniphila, Oxalobacter formigenes, Bacteroidales, Victivallales bacterium, Alistipes finegoldii, Bacteroides ilei, Lachnospira eligens, Bacteriophage Unclassified, Odoribacter splanchnicus, Haemophilus parainfluenzae, Sutterellaceae, Alistipes shahii, Bacteroides caccae, Odoribacter laneus, Alistipes communis, Acidaminococcus, Rikenellaceae bacterium, Bacteroides faecis, Collinsella, Streptococcus, Alistipes timonensis, Oscillibacter Unclassified, Lactobacillus salivarius, Haemophilus Unclassified, Acidaminococcus fermentans, Streptococcus salivarius, Muribaculum Unclassified, Clostridiales bacterium, Lactobacillus, Flavonifractor plautii, Ruminococcus lactaris, Subdoligranulum, Enterobacter hormaechei, Cyclospora cayetanensis, Bacteroides ovatus, Bacteroides finegoldii, Alistipes putredinis, Bacteroides cellulosilyticus, Bacteroides coprocola, Victivallis vadensis, Sutterella wadsworthensis, Duncaniella Unclassified, Acidaminococcus Unclassified, Dysosmobacter welbionis, Butyricimonas, Bacteroides intestinalis, Bilophila wadsworthia, Enterobacteriaceae, Lactobacillus gasseri, Klebsiella pneumoniae, Faecalitalea cylindroides, Porphyromonas asaccharolytica, Desulfovibrio desulfuricans, Lactobacillus paragasseri, Akkermansia Unclassified, Cryptobacterium curtum, Bacteroides vulgatus, Oscillibacter, Prevotella, Butyricimonas virosa, Bacteroides massiliensis, Prevotella lascolaii, Prevotella stercorea, Bacteroides plebeius, Prevotella Unclassified, Prevotella copri, Megasphaera Unclassified, Parabacteroides merdae, Escherichia, Escherichia coli, Streptococcus parasanguinis, Dialister invisus, Desulfovibrio fairfieldensis, Eubacterium maltosivorans, Veillonella parvula, Klebsiella, Acidaminococcus intestini, Desulfovibrio Unclassified, Paraprevotella clara, and Prevotella koreensis. In certain aspects, the one or more decreased bacteria are selected from a Streptococcus spp. In some aspects, the Streptococcus spp. is selected from the group consisting of Streptococcus mutans, Streptococcus salivarius, Streptococcus parasanguinis, and Streptococcus vestibularis. In some aspects, the one or more decreased bacteria are selected from the group consisting of Bacteroides massiliensis, Bacteroides stercoris, Prevotella copri, and Bacteroides plebeius. In some embodiments, an amount of Bacteroides massiliensis is decreased in the subject. In some embodiments, an amount of Bacteroides stercoris is decreased in the subject. In some embodiments, an amount of Prevotella copri is decreased in the subject. In some embodiments, an amount of Bacteroides plebeius is decreased in the subject. In some embodiments, the one or more bacteria selected from the group consisting of bacteria listed in Table 5 and Table 9 are decreased concurrently with the administration of the immune checkpoint inhibitor. In other embodiments, the one or more bacteria selected from the group consisting of bacteria listed in Table 5 and Table 9 are decreased in a subject after the subject has received an immune checkpoint inhibitor and has been determined to be unresponsive to the inhibitor.

The immune checkpoint inhibitor of the methods of decreasing intestinal bacteria can be any as described herein. In some embodiments, the immune checkpoint inhibitor is a PD-1 inhibitor described herein. The level or amount of the one or bacteria selected from the group consisting of bacteria listed in Table 5 and Table 9, or selected from the group consisting of a Streptococcus spp, Bacteroides massiliensis, Bacteroides stercoris, Prevotella copri, and Bacteroides plebeius, can be reduced within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more after the immune checkpoint inhibitor is administered to the subject. In some embodiments, the one or more bacteria can be reduced within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more before the immune checkpoint inhibitor is administered to the subject.

In some embodiments, the subject in which the effectiveness of the immune checkpoint inhibitor is increased has a cancer. The cancer can be any cancer that can be treated with an immune checkpoint inhibitor. In some embodiments, the cancer is lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, a lung cancer such as small cell lung cancer and non-small cell lung cancer, neuroblastoma/glioblastoma, ovarian cancer, skin cancer, liver cancer, melanoma, a squamous cell carcinoma of the mouth, throat, larynx, or lung, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer, testicular cancer, colon cancer, rectal cancer, prostatic cancer, or pancreatic cancer. In some embodiments, the cancer is an anti-PD-1 refractory cancer. In some embodiments, the cancer is melanoma. In some embodiments, the cancer is an anti-PD-1 refractory melanoma. As used herein, the word “refractory” refers to a cancer that is less responsive to a cancer treatment as compared to a control. In some embodiments, the control is a cancer in a subject or population that is clinically responsive to the cancer treatment. In some embodiments, a refractory cancer is one that is about 100%, about 90%, about 80%, about 70%, about 60%, or about 50% less responsive than a control. A “refractory cancer” includes a cancer that is resistant to treatment initially and a cancer that becomes resistant to treatment over time or during treatment.

Accordingly, also provided herein are methods of treating a cancer in a subject comprising increasing an amount of one or more bacteria in an intestine of the subject to a therapeutically effective amount, wherein the one or more increased bacteria are selected from the group consisting of bacteria listed in Table 4 and Table 8 and/or decreasing an amount of one of more bacteria in the intestine of the subject to a therapeutically effective amount, wherein the one or more decreased bacteria are selected from the group consisting of bacteria listed in Table 5 and Table 9, wherein the subject receives an immune checkpoint inhibitor. In some embodiments, the one or more increased bacteria are selected from the group consisting of Lachnospiraceae spp., Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis. In some embodiments, the one or more decreased bacteria are selected from the group consisting of a Streptococcus spp, Bacteroides massiliensis, Bacteroides stercoris, Prevotella copri, and Bacteroides plebeius. The increase or decrease of the aforementioned bacteria can occur within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18,24, 30, 36,48, 60 hours, 3,4, 5, 6,7, 8,9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more after the immune checkpoint inhibitor is administered to the subject. In some embodiments, the one or more bacteria can be reduced within 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months or more before the immune checkpoint inhibitor is administered to the subject.

Also provided herein are methods of predicting a subject's responsiveness to an immune checkpoint inhibitor comprising obtaining a stool sample from the subject and determining the presence or absence of one or more bacteria in the sample, wherein the one or more bacteria are selected from the group consisting of the bacteria listed in Table 4, Table 5, Table 8, and Table 9, wherein detection of the one or more bacteria listed in Table 4 or Table 8 predicts responsiveness and detection of the one or more bacteria listed in Table 5 or Table 9 predicts a lack of responsiveness. In some embodiments, methods of predicting a subject's responsiveness to an immune checkpoint inhibitor comprising obtaining a stool sample from the subject and determining the presence or absence of one or more bacteria in the sample, wherein the one or more bacteria are selected from the group consisting of Ruminococcus (Mediterraneibacter) torques, Ruminococcus (Mediterraneibacter) gnavus, Blautia wexlerae, Blautia hansenii, Eubacterium rectale, Actinomyces bouchesdurhonensis, Bacteroides massiliensis, Bacteroides stercoris, Prevotella copri, and Bacteroides plebeius, wherein detection of the one or more bacteria selected from the group consisting of Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis predicts responsiveness and detection of the one or more bacteria selected from the group consisting of Bacteroides massiliensis, Bacteroides stercoris, Prevotella copri, and Bacteroidesplebeius predicts a lack of responsiveness.

As used herein “responsiveness” refers to a beneficial outcome associated with an immune checkpoint inhibitor administration, such as a treatment of a cancer as compared to a control. In some embodiments, the beneficial outcome is a decrease or slowing in tumor growth, a decrease in tumor volume or size, a decrease in tumor number, a decrease in cancer recurrence, and/or a decrease in cancer metastasis, all as compared to a control subject or control or study population. In some embodiments the cancer is a refractory immune checkpoint inhibitor cancer, and in some embodiments, a refractory anti-PD-1 cancer. In some embodiments, the cancer is a refractory anti-PD-1 melanoma.

In some embodiments, a beneficial outcome is a decrease in an immune related adverse event associated with administration of an immune checkpoint inhibitor as compared to a control subject or control or study population. The immune related adverse event can be selected from the group consisting of: pneumonitis, arthritis, hepatitis, adverse cutaneous side effects (e.g., skin and subcutaneous disorders including rash, pemphigoid, and pruritus), adrenal insufficiency, muscle inflammation, thyroid abnormality (including hyperthyroidism, hypothyroidism, thyroiditis), colitis, nephritis, and adverse neurological side effects (e.g., Guillain-Barré syndrome, encephalitis, and myasthenic syndrome). Accordingly, in some embodiments, the methods decrease or treat an immune checkpoint inhibitor induced pneumonitis. In some embodiments, the methods decrease or treat an immune checkpoint inhibitor induced arthritis. In some embodiments, the methods decrease or treat an immune checkpoint inhibitor induced rash. In some embodiments, the methods decrease or treat an immune checkpoint inhibitor induced pemphigoid. In some embodiments, the methods decrease or treat an immune checkpoint inhibitor induced pruritus. In some embodiments, the methods decrease or treat adrenal insufficiency. In some embodiments, the methods decrease or treat an immune checkpoint inhibitor induced muscle inflammation. In some embodiments, the methods decrease or treat an immune checkpoint inhibitor induced hyperthyroidism. In some embodiments, the methods decrease or treat an immune checkpoint inhibitor induced hypothyroidism. In some embodiments, the methods decrease or treat an immune checkpoint inhibitor induced thyroiditis. In some embodiments, the methods decrease or treat an immune checkpoint inhibitor induced colitis. In some embodiments, the methods decrease or treat an immune checkpoint inhibitor induced nephritis. In some embodiments, the methods decrease or treat an immune checkpoint inhibitor induced Guillain-Barré syndrome. In some embodiments, the methods decrease or treat an immune checkpoint inhibitor induced encephalitis. In some embodiments, the methods decrease or treat an immune checkpoint inhibitor induced myasthenic syndrome.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes.

### EXAMPLES

### Example 1

### Methods

### Patient Enrollment and Assessments, Clinico-Demographic Variables, Survival Endpoints and Treatment-Emergent Adverse Events (TRAE)

Patient enrollment: Patients with unresectable stage III or IV melanoma treated at the University of Pittsburgh's Hillman Cancer Center who were receiving frontline anti-PD-1 therapy for treatment of advanced disease were eligible. Patients were treated with single-agent anti-PD-1 immunotherapy (nivolumab, pembrolizumab or investigational anti-PD-1) singly or pembrolizumab in combination with peg-IFN in the context of a separate clinical trial, the results of which have been previously published (HCC 13-105, IRB approval number CR19090075-002) (Davar D. et al., 2018). Stool samples were banked under University of Pittsburgh Institutional Review Board692 approved banking protocols (HCC 96-099 and 20-019, IRB approval numbers MOD19080226-004 and STUDY20010266 respectively). Radiographic response to therapy while receiving PD-1 inhibitor treatment was determined by treated investigators and assessed using response evaluation criteria in solid tumors (RECIST) v1.1 (Davar, D., et al. 2018). Clinical response to therapy was assessed at each visit. Progression was defined based on first documented clinical and/or radiographic progression and confirmed in all instances.

Clinico-demographic variables: Body mass index (BMI), neutrophil-lymphocyte ratio (NLR) and lactate dehydrogenase (LDH) were based on values obtained immediately prior to therapy or on day of anti-PD-1 initiation. PPI exposure was defined as exposure to omeprazole, esomeprazole, lansoprazole, deslansoprazole, pantoprazole, rabeprazole taken by patients for at least 30 days preceding date of stool sample collection.

Survival endpoints: Progression-free survival was defined as time from start of therapy to first confirmed clinical and/or radiographic progression. Overall survival was defined as time from start of therapy to date of death. Patients were censored as of the date of last contact.

Treatment-emergent adverse events (TEAE): TEAEs were defined as any clinical and/or laboratory event that occurred following initiation of anti-PD-1 therapy that based on investigator assessment was definitely linked to the administration of anti-PD-1 therapy. AEs were considered irAEs based on mechanism of action and a prespecified list of terms developed by study investigators and grouped under the following broader terms: pneumonitis, colitis, hepatitis, nephritis, arthritis, thyroid (including hyperthyroidism, hypothyroidism, thyroiditis), adrenal (adrenal insufficiency), dermatologic (skin and subcutaneous disorders including rash, pemphigoid, and pruritus), vitiligo and neurologic (Guillain-Barré syndrome, encephalitis, and myasthenic syndrome). Infusion reactions without immunologic etiology were not included.

### DNA Extraction, Shotgun Metagenomic Sequencing, Metatranscriptomic Sequencing and Analysis

Total metagenomic DNA was extracted from stool samples using the MO BIO PowerSoil DNA Isolation Kit (MO BIO Laboratories, Carlsbad, CA, USA) and Epmotion 5075 liquid handling robot (Eppendorf). The DNA library was prepared using the Nextera DNA Flex Library Prep Kit, quantified using Qbit, and sequenced on the NovaSeq System (Illumina, Inc, San Diego, CA, USA) using the 2×150 base pair (bp) paired-end protocol.

Quality trimming and adapter clipping of raw reads for each metagenomic sample was carried out using Trimmomatic 0.36 (Bolger, A. M., et al. 2014). The reads were then aligned against the human genome with Bowtie2 v2.3.2. (Langmead, B. & Salzberg, S. L. 2012) and unaligned (non-host) reads were then assembled using MEGAHIT v1.2.9 (Li, D., et al., 2015, Li, D., et al. 2016). Assembly contigs smaller than 500 bp were discarded. For the 94 samples, the mean sequencing depth (already discounting host reads) was 10.14 Gbp±4.95 Gbp, yielding a mean assembly rate of 81.94%±5.24%.

Taxonomic classification of contigs was achieved by k-mer analysis using Kraken2 (Wood, D. E., Lu, J. & Langmead, B. 2019) with a custom 96-Gb Kraken2 database built using draft and complete genomes of all bacteria, archaea, fungi, viruses, and protozoa available in the NCBI GenBank in April 2020, in addition to human and mouse genomes. Functional annotation of contigs was done ab initio with Prokka v1.14.6 (Seemann, T 2014). For evaluating the sequencing depth of each contig, reads used for assembly were then aligned back to the assembly contigs. The unassembled reads for each sample were classified individually using Kraken2 on the same database. Taxonomy was expressed as the last known taxon (LKT), being the taxonomically lowest unambiguous classification determined for each query sequence, using Kraken's confidence scoring threshold of 5e-06 (using the —confidence parameter). The relative abundance for each LKT within each sample was obtained by dividing the number of bp covering all contigs and unassembled reads pertaining to that LKT by the total number of non-host base pairs sequenced for that sample. Relative abundances were expressed in PPM.

For comparisons between samples, ordination plots were made with the t-distributed stochastic neighbor embedding (t-SNE) algorithm using the uwot package in R (github.com/jlmelville/uwot) and the ggplot2 library. Permanova values were obtained using the adonis function of the vegan package, with default (999) permutations and pairwise distances calculated using Bray-Curtis distance. Heatmaps were drawn using the ComplexHeatmap package for R (Gu, Z., Eils, R. & Schlesner 216).

All code used for shotgun sequencing analysis can be found within the in-house JAMS_BW package, version 1.5.7, publicly available on GitHub (github.com/johnmcculloch/JAMS_BW). Meta-analysis of microbiome associated with the response was done as follows. Individual Rs were first analyzed using the non-parametric t-test. Then p-values and ratios were combined using Fisher's method and R package meta (github.com/guido-s/meta). Resultant data were visualized using the cladogram feature from package LEfSe (Segata, N., et al. 2011).

### Transkingdom Network Analysis of Multiomic Data

Network Reconstruction: To create a statistical model of robust interactions between the different players, this study created a transkingdom network. Microbial (taxa and genes) and host (genes and phenotypes) nodes significantly different between NPs and Ps were first selected. Next, Spearman rank correlation was calculated between all pairs of nodes. To keep robust relationships independent of a particular group, within-omics interactions were selected if they had the same sign of correlation in: (i) Early Ps, (ii) Early NPs, (iii) Late NPs and if they satisfied principles of causality (i.e., satisfied fold-change relationship between the two partners in NPs vs. Ps). For between-omics interactions, an additional criterion was applied that checked for sign of correlation calculated using all samples should match that of the three within-group correlations. The combined p-value (CP) for meta-analysis of within-group correlations was calculated based on Fisher's z transformation of correlations (metacor in R package meta v4.9-7). For within-omics edges the cutoffs were as follows—Phenotypes: CP<5%, false discovery rate (FDR)<10%; host genes: within-group p-values<35%, CP<10%, FDR<15%; LKTs: within-group p-values<30%, CP<10%, FDR<15%; microbial genes: within-group p-values<3%, CP<1%, FDR<5%. Connections of microbes (taxa/microbial genes) and host genes with phenotypes had FDR<5% and 25%, respectively. Connections of taxa and microbial genes with host genes had FDR<15% and 5%, respectively, whereas taxa and microbial gene connections had within-group p-values<0.25% and FDR<1%. The resulting TK network was visualized with Cytoscape Software 2.6.3 (103). The degree and bipartite betweenness centrality (Bi-BC) were calculated using R 3.6 and compared between the beneficial and detrimental LKTs (two-tail Mann Whitney test p-value<5%).

Determining the Optimal Cutoff Point for Biomarkers with Continuous Values

Analysis was done using the Evaluate Cutpoints software and as described previously (Ogluszka, M., 2019). Briefly, the coxph function from the survival package was used to fit Cox proportional hazard model to the binary outcome (overall survival: dead/alive; progression-free survival: progressed/not) and continuous (overall or progression-free survival time and biomarker value) covariates. Cutpoint was then computed with the cutp function (survMisc package) and the samples were categorized into high and low groups based on biomarker values. Kaplan-Meier plot was generated using a combination of survival, ggplot2 and plotly packages. Finally, uni- and multi-variate Cox regression analyses for categories was done using the coxph function from survival package and hazard ratio, confidence intervals and p-values were calculated.

### Machine Learning Methods

Machine learning analyses were performed on taxonomic counts that were batch corrected by study using ComBat (Johnson, W. E. 2007). Each study was then balanced by randomly removing either responder or non-responder samples so that equal numbers of responders and non-responders were obtained. Machine Learning models were produced for each study with the R package caret (CRAN. R-project.org/package=caret) using a training set of 70% of the count data, with the remaining 30% used to evaluate model's performance. Separately, leave one out validation was performed. Accuracy and AUC were measured using a bootstrap analysis, with the results from the case with median accuracy used. For comparison, several machine learning methods were tested—random forest₁₀₅, support vector machine (SVM) (Boser, B. E., et al., 1992), generalized linear models (GLM) (Nelder, P. M. J. A. 1989) and dropouts meet multiple additive regression trees (DART) (Vinayak, R. K. & Gilad-Bachrach, R. 2015).

### Transcriptomic Analyses of Intestinal Tissues Using Next Generation RNA Sequencing of Stool Samples.

Fecal RNA was isolated with Epmotion 5730 using MO BIO's PowerFecal kit, Qiagen. Sequencing libraries were prepared using Illumina TruSeq Stranded Total RNA sequencing kit following depletion of bacterial ribosomal RNA with RiboZero (Illumina) kit. Libraries were quantified using Qubit and sequenced using Nextseq 500. Sequencing reads quality and adaptor trimmed with Trimmomatic 0.33 (Bolger, A. M., 2014), and analyzed using RSEM package. Resultant count files were further analyzed with in R 4.04. Briefly, genes with counts less than 5 reads, genes encoding ribosomes, ribosomal and mitochondrial genes were removed, counts were log transformed and quantile-normalized. P-values were calculated using Mann-Whitney U-test and visualized using ggplot2 R package. Significant genes were uploaded to Ingenuity Pathway studio, upstream regulator analysis output was used to construct a network using Cytoscape 3.8.0 (Shannon, P., et al. 2003). For cell prediction analysis using GSEA we used a gmx file containing cell-specific gene profiles that we derived from the Immgen database (www.immgen.org). The GSEA analysis was done in R using fgsea package. For difference between NPs and Ps (y axis of the plot) we used ranked values of NPs/Ps ratios. For the prediction of the cell abundance (x axis of the plot) we used ranked values of the individual samples. The resultant values of the leading edge GSEA analysis were used to calculate ratios (for y axis of the plot) to present differences between responders and non-responders and presented as average values of leading edge analysis for all samples to visualize the abundance of the predicted cell type, analogous to a standard MA plot.

### Meta-Analysis Methodologies

Data was analyzed using two meta-analysis methods: Fisher's method for combining p-values and random effects model. Random effects model meta-analysis was calculated using R package meta using default parameters. Taxa with heterogeneity p-values of greater than 0.1 were considered. Fisher's combined p-value was calculated as following: Results of individual analysis of all datasets were compared. Taxa that agreed in directionality of the NPs vs Ps differences in more than four out five datasets were considered. In case, when taxa in one of five datasets failed to agree in directionality, its p-values were assigned as 0.999 and ratio as one. χ² and the combined p-value were calculated as described previously (Rodrigues, R. R., 2018). Data was visualized using heatmap or volcano plot (R 4.04).

### Reference-Independent Binning Analysis of Metagenomic Data From Combined Cohorts

Sequencing reads were quality trimmed using Trimmomatic 0.3396, host RNA was removed using Bowtie2 v2.3.2. (Langmead, B. & Salzberg, S. L. 2012). Reads for all samples from all five cohorts were combined into one large file and larger sequence contigs were assembled using MEGAHIT 1.2.9 (Li, D., et al. 2015; Li, D., et al. 2016). Reads longer than 1,000 bp were used in analysis. Contigs were indexed using bwa package. and reads from individual samples were aligned to the contigs. The resultant dataset was used to calculate clusters using Phenograph R package and tSNE plot. To reduce overlap of clusters, contigs were subdivided into taxonomic units using Kraken package prior to application of Phenograph clustering tool. Clusters were merged into a single fasta files and analyzed again using Kraken. The resultant clusters we considered as independent functional taxonomical units (FTU). To perform comparison between FTUs within specific taxa for every FTU we created a taxa-gene dataset, where existence of the gene was remarked by number “1” and absence by “0”. Beneficial and detrimental taxa were identified using statistical tools described above and top 30% and bottom 30% of taxa were used for statistical comparisons. Significant data were visualized using Cytoscape.

### Clustering of Enteric Microbiotypes Metagenomic Data and American Gut Project (AGP) Data

Stool AGP data was used for the analysis. Only read 1 was used in the analysis. The data was pooled together with 16S data from above mentioned cohorts, Quality trimmed data were combined into one file, and unique sequences with reads more than 10 per read were identified and used as a reference. The unique reads were indexed with bwa; and sequences from individual samples were mapped back to those reads. The data, similar to the analysis above, was clustered using Phenograph R package; the most abundant sequence of the cluster was classified using RDP algorithm in Mothur package; and the data was assigned to the whole cluster. The resultant dataset was filtered to include at least 5,000 reads per samples and clustered using Phenograph R package and t-SNE into bins (or tentative microbiotypes). To determine the beneficial or detrimental status of the microbiotype, every OR was calculated for every cluster. Once the beneficial and detrimental clusters were defined, we used LDA analysis (Lefse package) or ANOVA analysis to identify taxonomic composition of those groups.

### Example 2

### Patients and Treatment

Stool samples were collected from 94 melanoma patients treated with anti-PD-1 under a University of Pittsburgh Institutional Review Board-approved banking protocols (HCC 96-099 and 20-019). Stool samples were collected either pre-treatment or within four months of starting anti-PD-1 (63 patients, Pittsburgh early sample cohort) or after more than four months (4-41 months) from start of anti-PD-1 (31 patients, Pittsburgh late sample cohort). The early sample cohort with samples collected either before treatment start or by the time of the first staging visit was considered representative of baseline microbiome due to the observations that the fecal microbiome of patients treated anti-PD-1, unlike anti-CTLA-4 or chemotherapy, is stable at the early times after therapy initiation (Vetizou, M., et al. 2015; Routy, B., et al. 2018; Derosa, L., et al. 2020; Viaud, S., et al. 2013). Patient demographics and clinical characteristics are detailed in Table 1. The impact of select clinical characteristics upon overall survival (OS) and progression-free survival (PFS) was evaluated using Cox regression hazards model (Table 2). The multivariate analysis of the early sample cohort showed that body mass index and NLR were independently and negatively associated with OS, while use of proton pump inhibitors (PPI) and NLR were negatively associated with PFS. PD-1 naïve patients received either anti-PD-1 singly (nivolumab, pembrolizumab or investigational PD-1 inhibitor) or in combination with pegylated interferon α2b (pegIFN) in the context of a separate clinical trial (HCC 13-105) (Davar, D., et al. 2018). The early sample cohort included 49 patients treated with anti-PD-1 alone and 14 patients treated with pembrolizumab/pegIFN. The late sample cohort comprised 31 patients, 24 of whom received anti-PD-1 alone and 7 received pembrolizumab/pegIFN (Table 1). While 82% patients (78/94) were treated in the front-line setting, 17% patients (16/94) received prior therapy with CTLA-4 inhibitor, BRAF/MEK inhibitors, cytokines and/or vaccines. Grade 1-4 irAEs were observed in 62% patients (58/94).

The data from the Pittsburgh melanoma cohort were also combined in a meta-analysis with four previously reported cohorts of anti-PD-1 treated melanoma patients summarized in Table 3 (Gopalakrishnan, V., et al. 2018; Matson, V., et al. 2018; Frankel, A. E., et al. 2017; Peters, B. A., et al. 2019).

### Example 3

### Compositional Differences in the Fecal Microbiome Predicts Objective Response and PFS in a New Cohort of PD-1 Treated Melanoma Patients

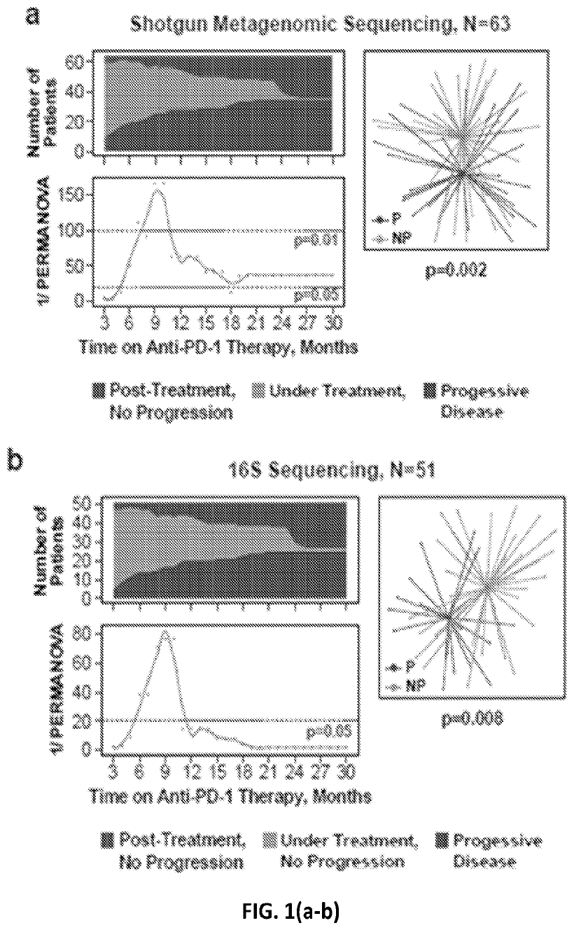

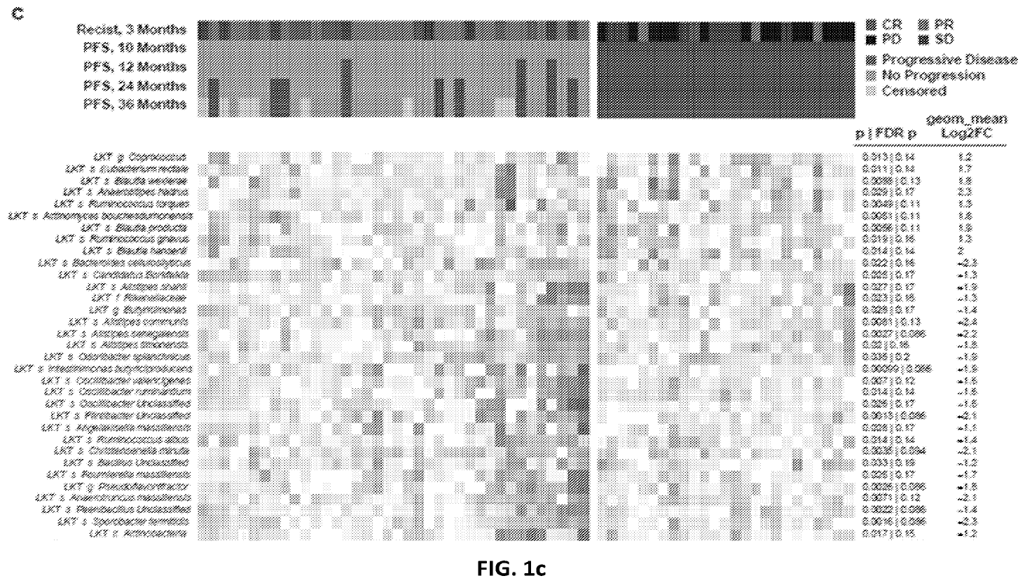

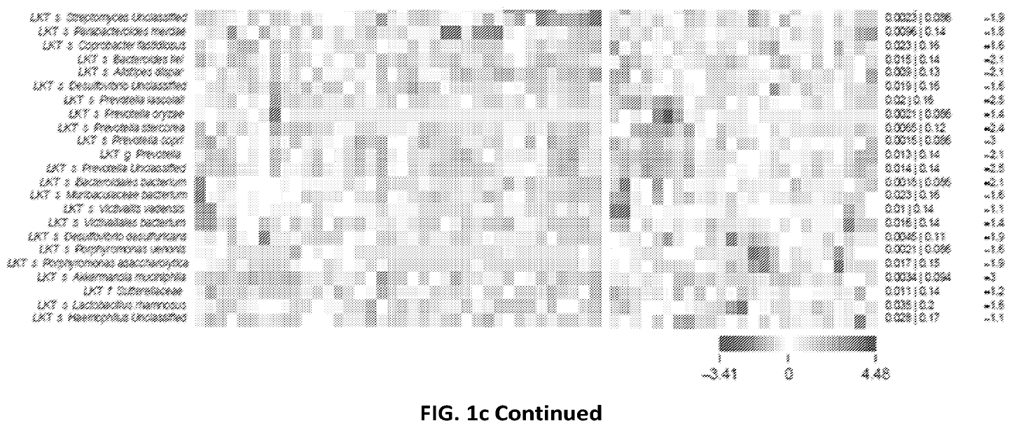

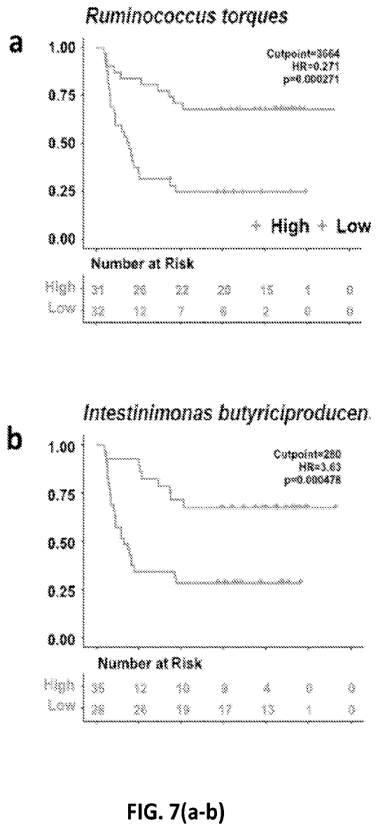

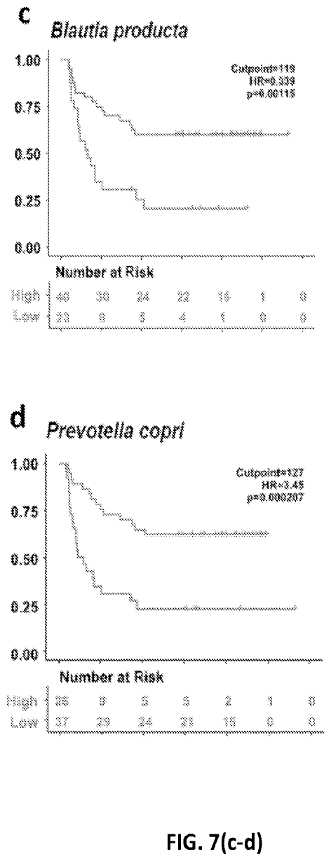

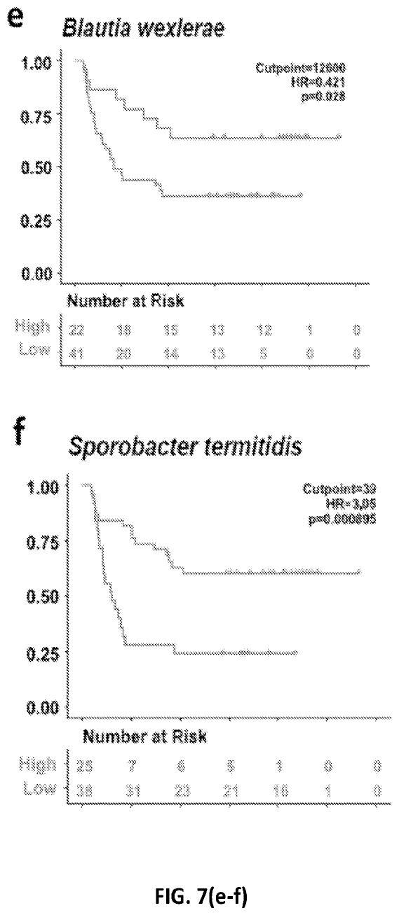

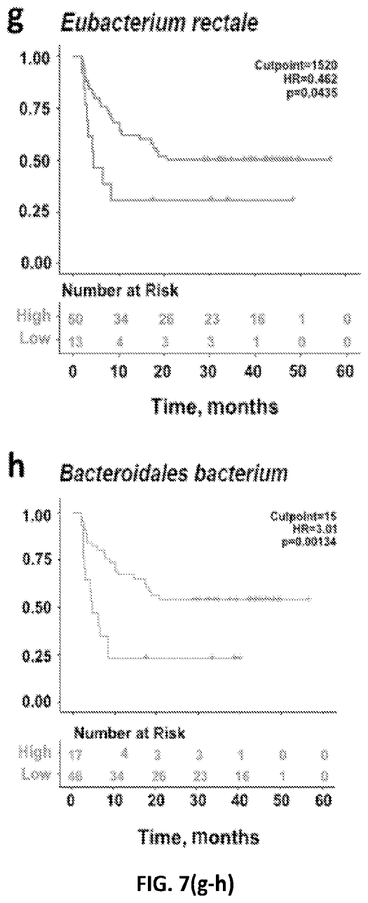

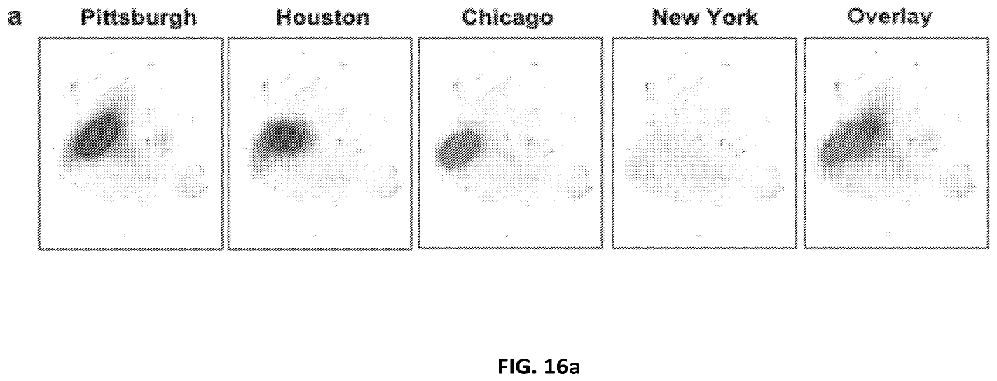

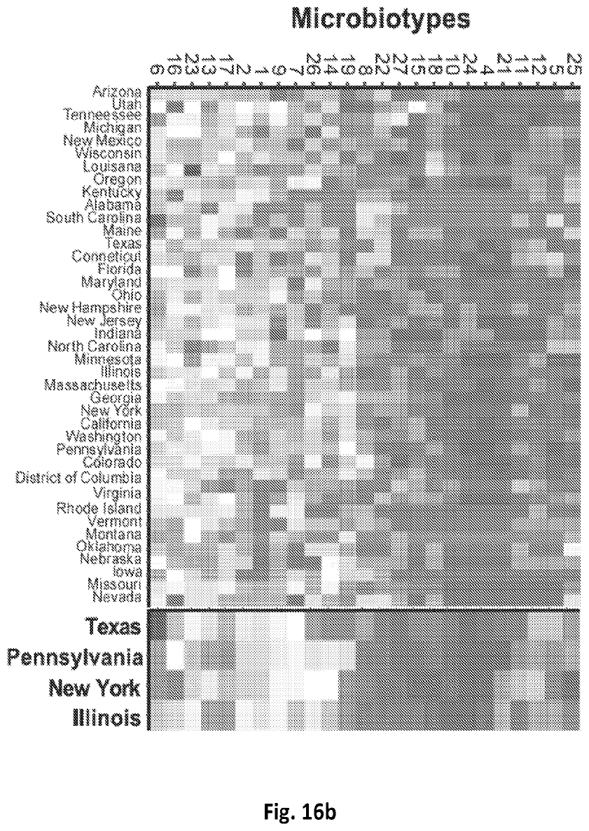

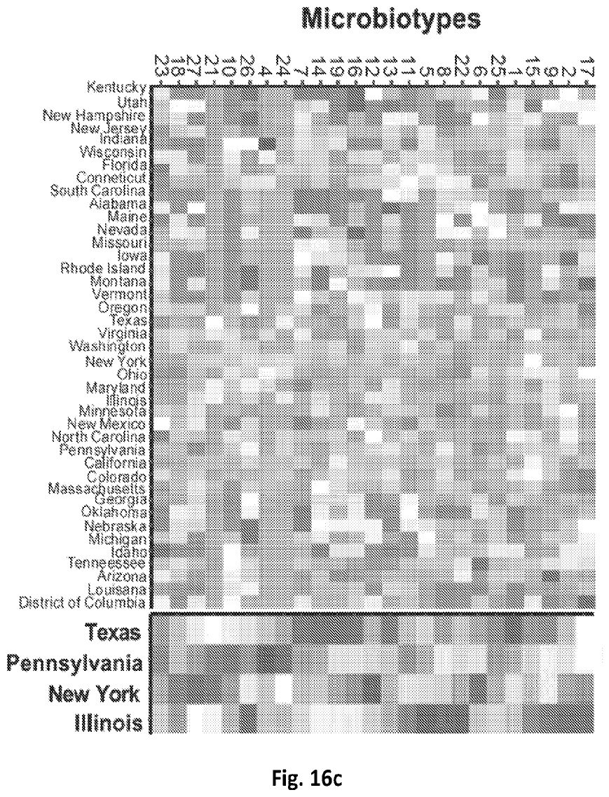

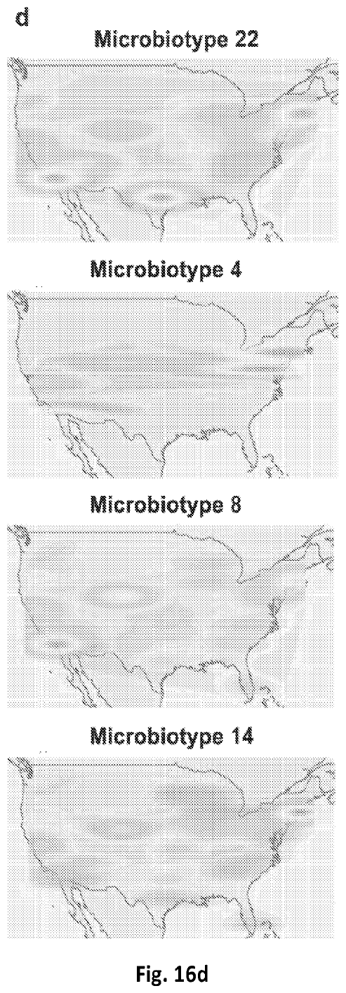

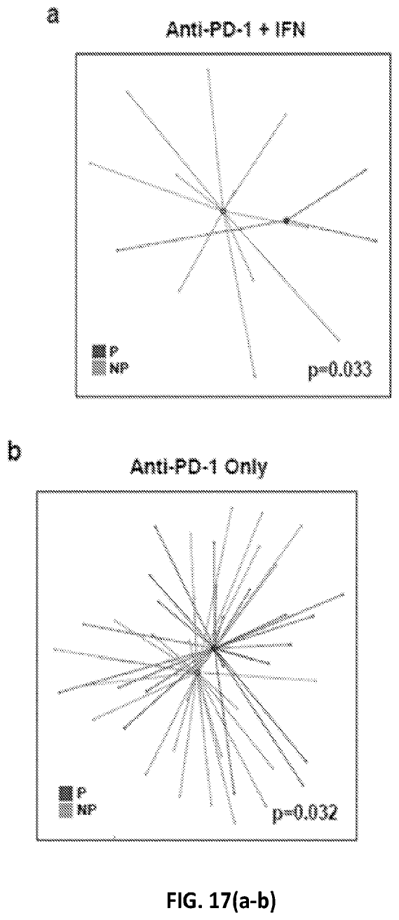

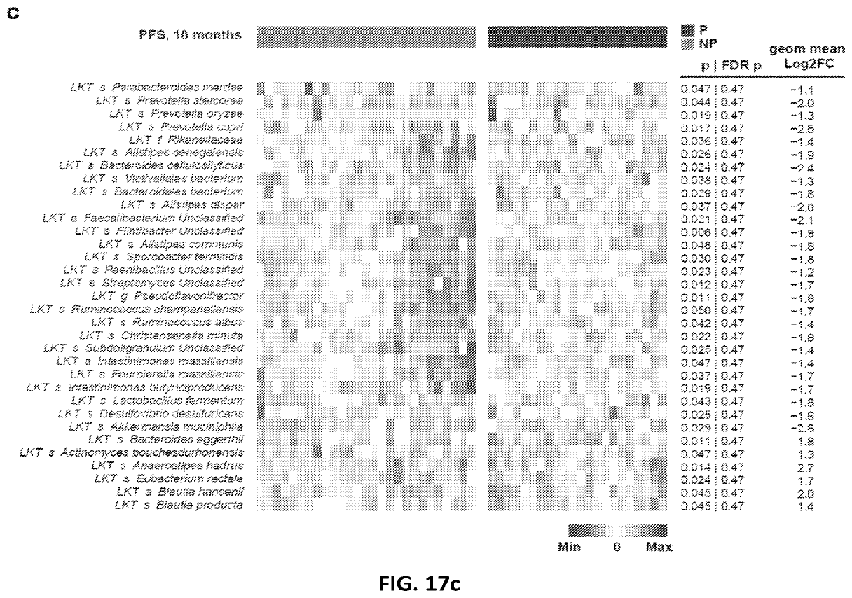

Objective radiographic response to therapy was assessed using RECIST v1.1 every 3 months (Eisenhauer, E. A., et al. 2009). To estimate the timepoint at which baseline intestinal microbiome composition (collected at pretherapy or up to 4 months) had maximal predictive power of clinical outcome, we computed PERMANOVA p-values of taxonomic compositional abundances using shotgun (FIG. 1a) and 16S rRNA gene amplicon (FIG. 1b) sequencing between non-progressors (NPs) and progressors (Ps) at various timepoints. The baseline intestinal microbiota composition reflected maximal separation between the clinical outcomes at 9-10 months, whether by shotgun (FIG. 1a, p=0.006) or 16S (FIG. 1b, p=0.013) sequencing. Identification of responder (R) or non152 responder (NR) patients by RECIST 1.1 [partial or complete response (PR or CR)] at 3 months resulted in a PERMANOVA p of 0.027 and 0.185 for shotgun and 16S sequencing, respectively (FIG. 1a, 1b). To identify differentially abundant taxa in NPs versus Ps at 10 months, metagenomic sequencing data of baseline fecal microbiota samples were compared using Mann-Whitney U test (FIG. 1c). The most differentially abundant species in NPs were Ruminococcus (Mediterraneibacter) torques, Blautia producta, Blautia wexlerae, Blautia hansenii, Eubacterium rectale, Ruminococcus (Mediterraneibacter) gnavus, and Anaerostipes hadrus. The fecal microbiome of Ps exhibited increased abundance of Prevotella spp., Oscillibacter spp., Alistipes spp. and Sutterellaceae spp. For each differentially abundant bacterial taxon, we evaluated the optimal cutoff of abundance by fitting Cox proportional hazard model to the clinical outcome (progression or not), PFS time, and fecal bacterial abundance and calculated the Kaplan Meier curves (representative plots for bacterial species most significantly correlated either with non-progression or progression at 10 months are shown in FIG. 7). Differential microbiota composition between Ps and NPs was compared between patients treated either with anti-PD-1 alone (49 patients) or in combination with pegIFN (14 patients) (FIGS. 17a and 17b). The gut microbial compositions associated with progression or non-progression in the combined analysis of the 63 patients treated with either anti-PD-1 alone or anti-PD-1 and pegIFN, were mostly similar (FIG. 1c and FIG. 17c). Therefore, the addition of pegIFN did not significantly alter metagenomic results while increasing sample size and significance of the observed differences.

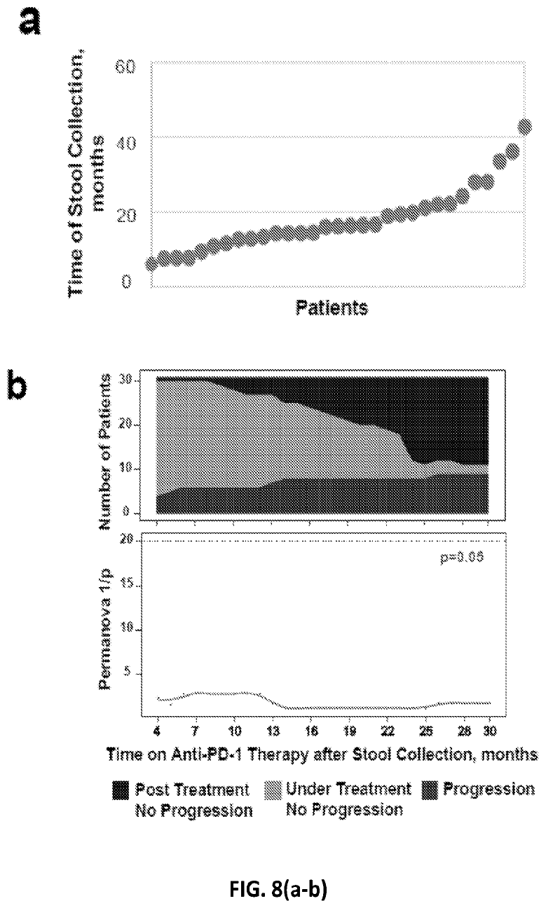

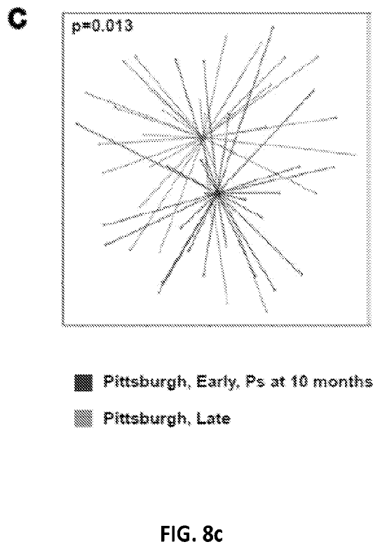

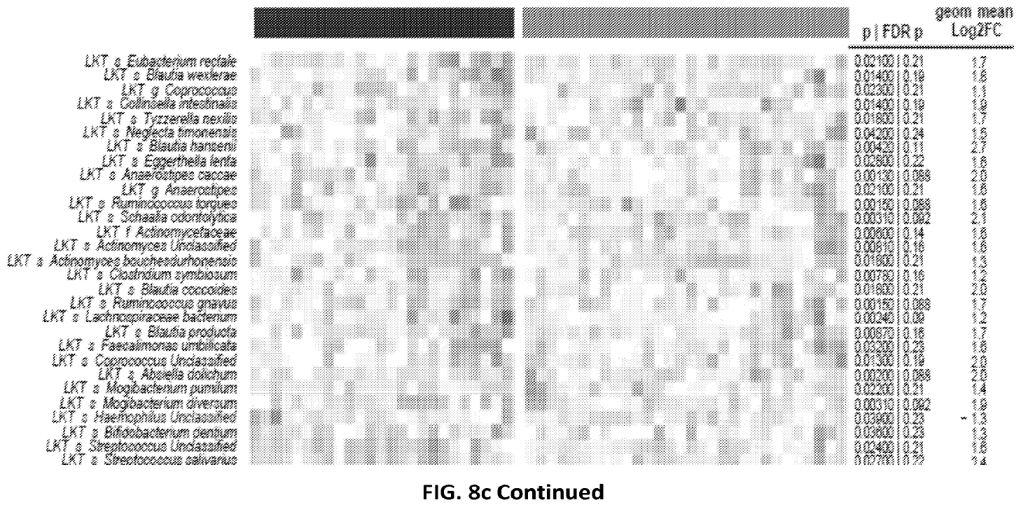

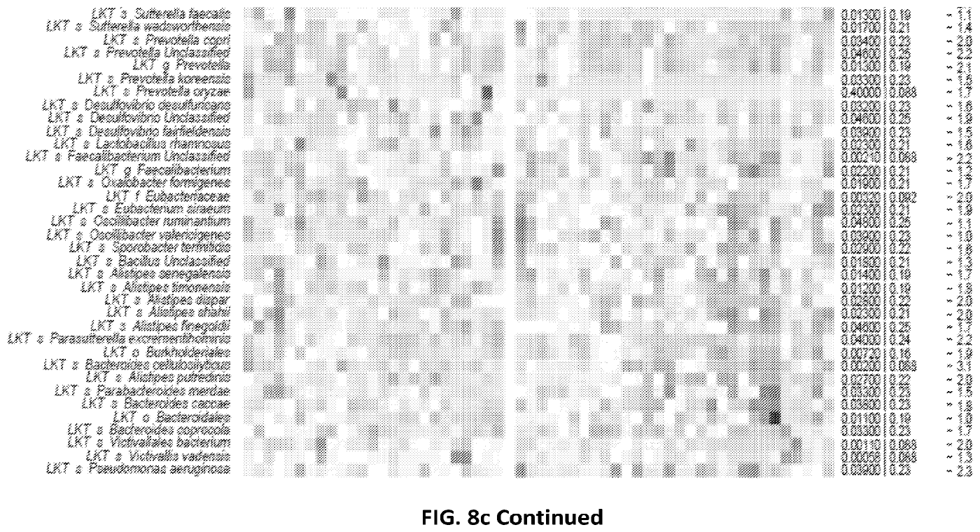

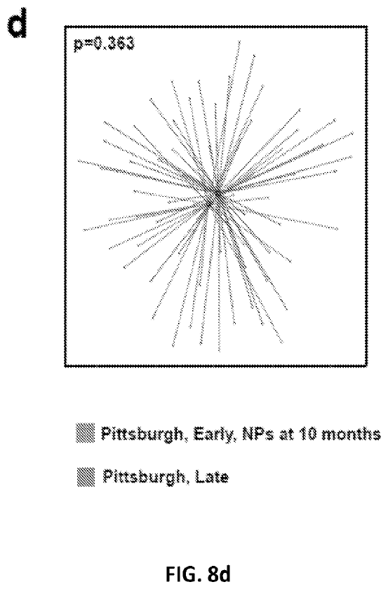

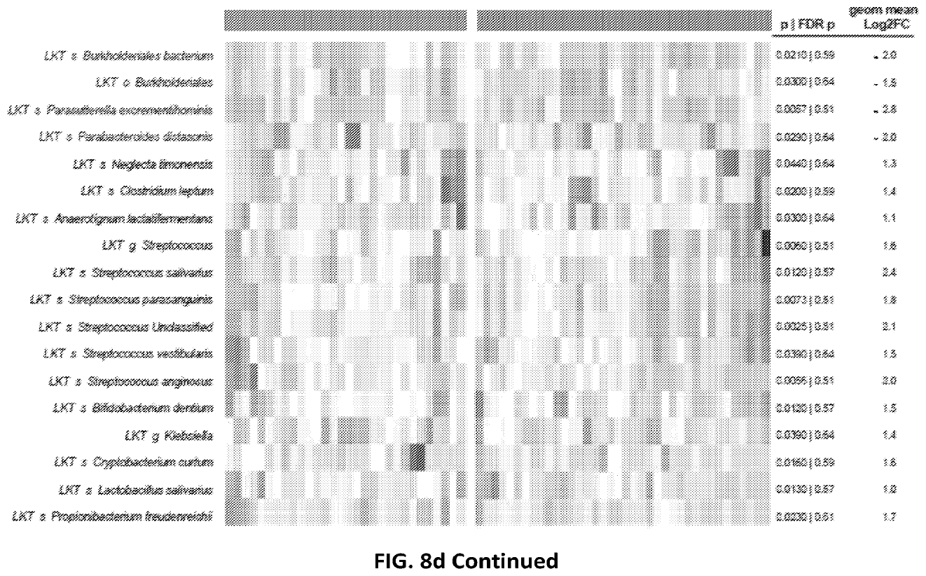

To evaluate the role of the microbiome during the entire course of therapy, this study separately evaluated the composition of the fecal microbiome (stool samples collected at 4 to 41 months from start of anti-PD-1) obtained from the late sample cohort (FIG. 8a). All but one patient in the late sample cohort were NPs at the time of collection, limiting our ability to define the role of on-treatment intestinal microbiome composition upon response to anti-PD-1 over the duration of therapy (FIG. 8b). The microbiome composition collected from long-term NPs did not predict subsequent failure of anti-PD-1 observed in a subset of these patients (FIG. 8b). Long-term NPs of the late sample cohort exhibited a fecal microbiome composition that was significantly distinct from Ps in the early sample cohort (FIG. 8c, p=0.013), albeit largely similar to NPs (10 months) in the early sample cohort (FIG. 8d, p=0.363). The fecal microbiota of long-term NPs in the late sample cohort differed from Ps in the early sample cohort due to increased abundance of Lachnospiraceae spp. and decreased abundance of Bacteroidetes spp. of the genera Prevotella, Alistipes and others. Beneficial microbiome signatures are thus largely preserved over time in long-term NPs (heatmaps in FIG. 1c and FIG. 8c).

### Example 4

Time-to-Event Analyses Identified Additional Taxa Associated with Clinical Response in the New Cohort of PD-1 Treated Melanoma Patients

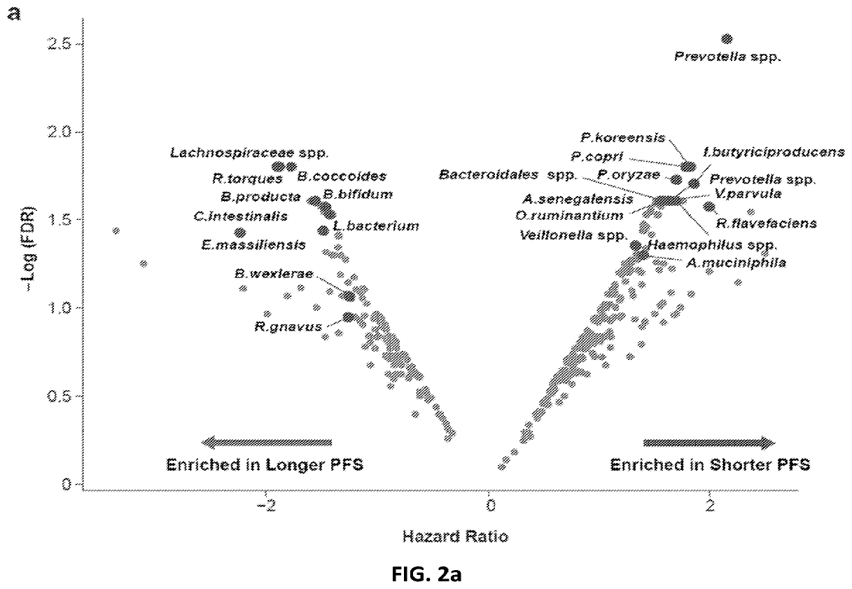

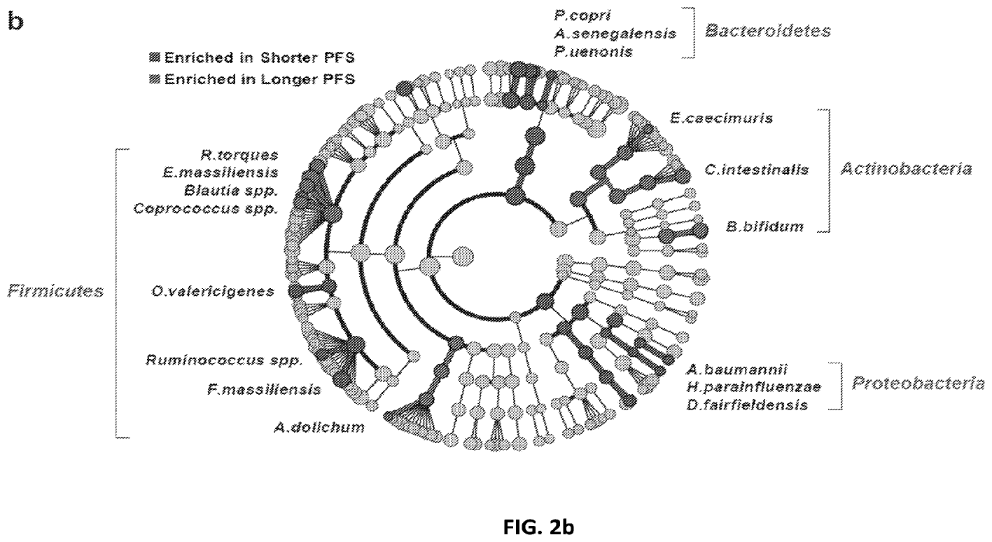

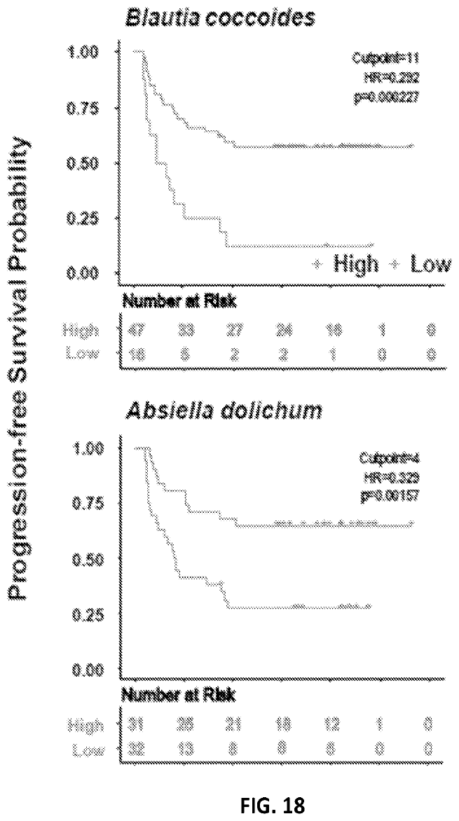

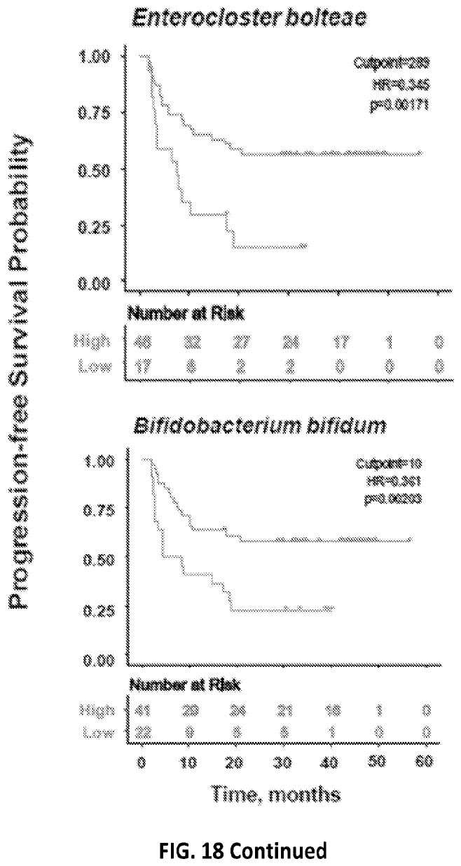

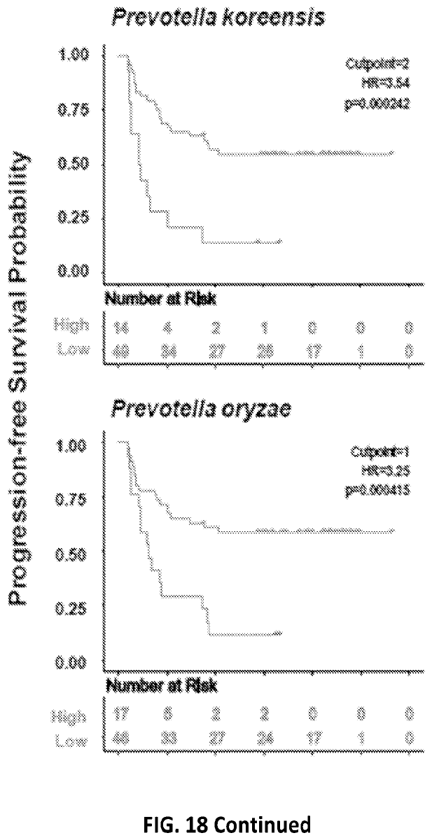

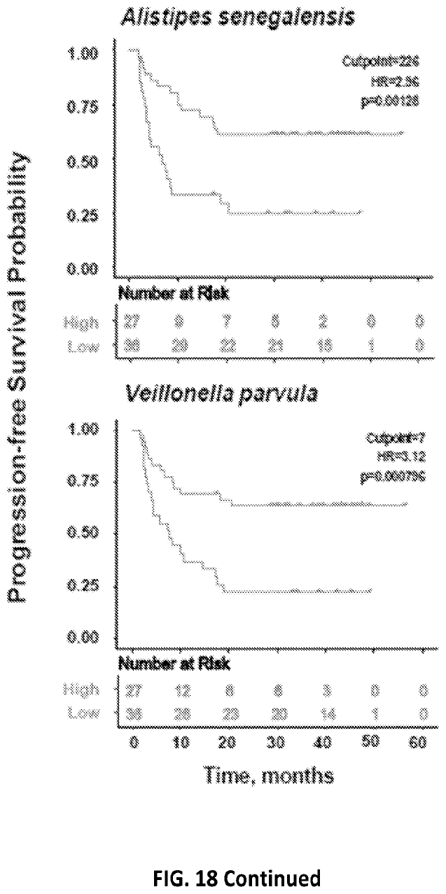

Considering the longitudinal impact of the gut microbiome upon clinical outcome to anti-PD-1, a separate analysis was performed wherein the beneficial or detrimental impact of bacterial species was evaluated by determining fecal abundance cut points for all organisms and calculating hazard ratios (HR) in relation to PFS using Evaluate Cutpoints (Ogluszka, M., 2019). This approach identified the same taxa previously described in FIG. 1c, along with many additional taxa associated with either improved or decreased PFS. These taxa are summarized in a volcano plot [HR vs. false discovery rate (FDR)] in FIG. 2a and in a cladogram in FIG. 2b. The complete lists of significant taxa associated with improved and decreased PFS are tabulated in Tables 4 and 5, respectively. The organisms with a beneficial role (longer PFS) include Coriobacteriaceae, Lachnospiraceae, Bifidobacteriaceae and Erysipelotrichaceae families, and those with a detrimental role (shorter PFS) include members of families of the Bacteroidetes phylum such as Prevotellaceae and Rikenellaceae as well as select members of Firmicutes phylum such as Streptococcus spp. (Gopalakrishnan, V., et al. 2018; Matson, V., et al. 2018). Kaplan Meier plots for selected significant taxa associated with either improved or decreased PFS are shown in FIG. 18.

### Example 5

Bacterial Metagenomic and Host Transcriptomic Analyses Identify Unique Pathways Associated with Beneficial and Detrimental Intestinal Microbiome Signatures in Melanoma Patients Treated with Anti-PD-1

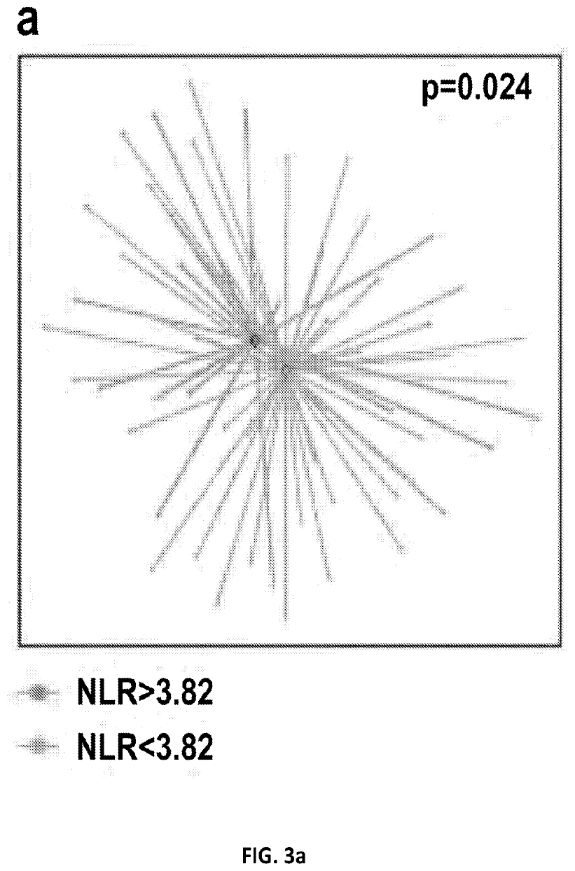

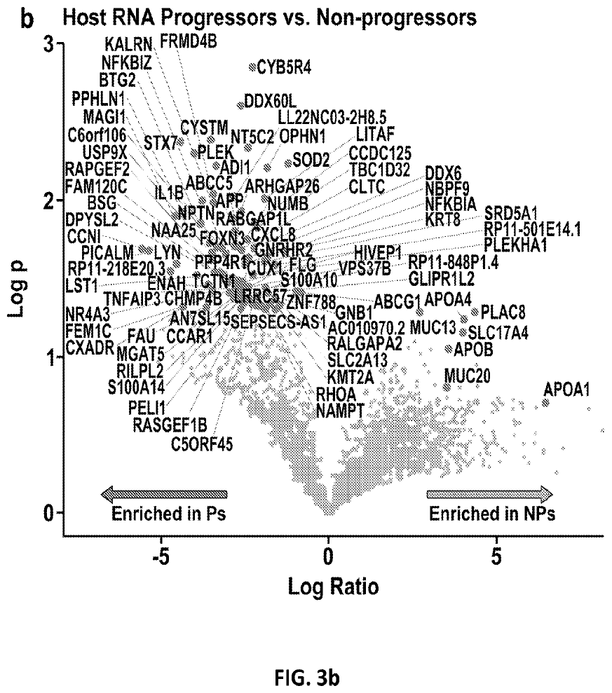

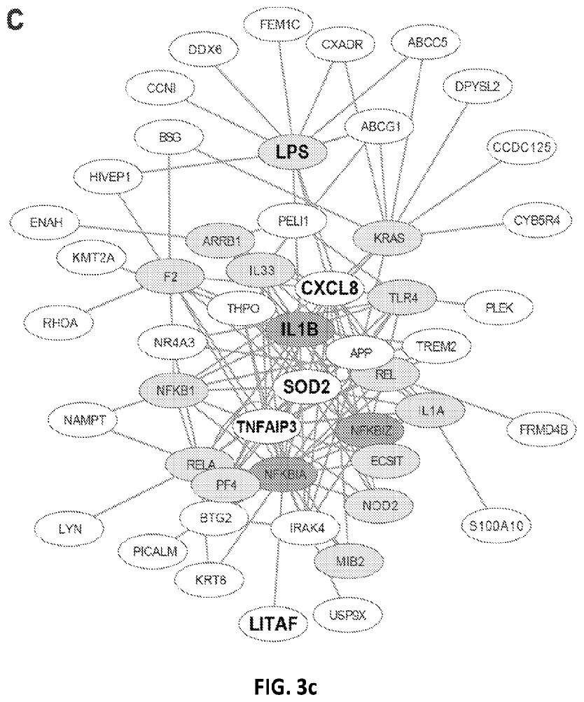

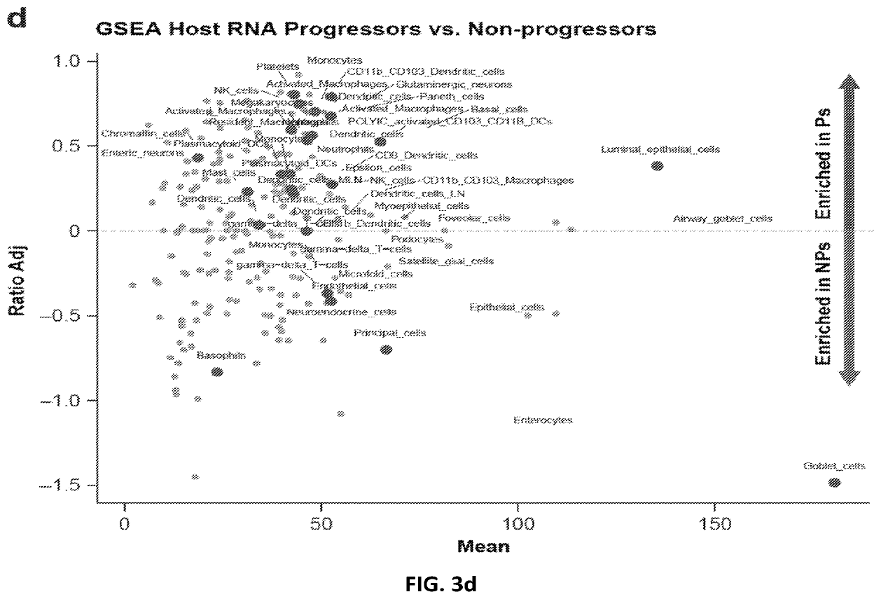

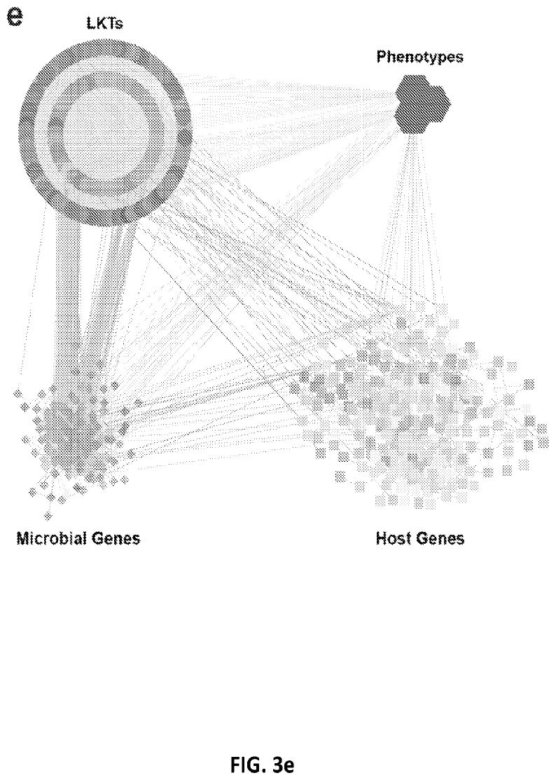

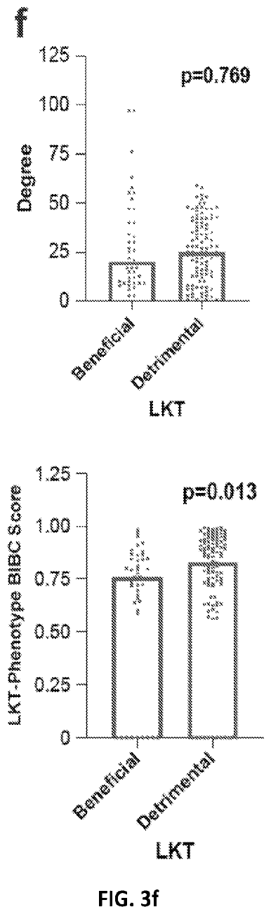

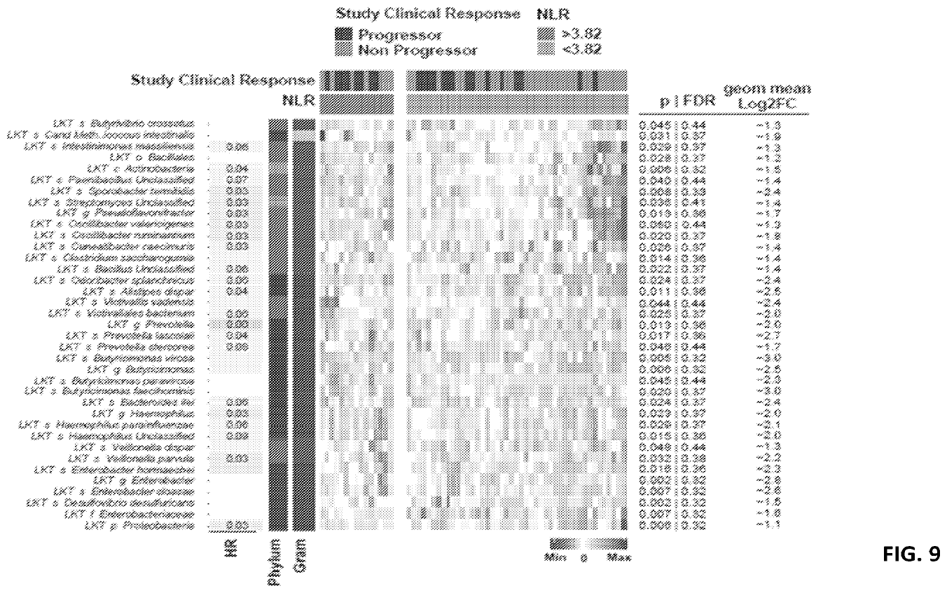

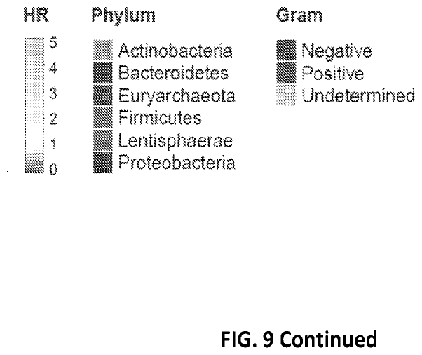

The next experiment sought to evaluate the impact of gut microbiome composition upon NLR—a biomarker of systemic inflammation with well characterized adverse impact upon response to anti-PD-1 in melanoma and other solid cancers (Capone, M., et al. 2018; Valero, C., et al. 2021; Ascierto, P. A., et al. 2019). This experiment first confirmed that increased NLR correlated with worse clinical outcome in the Pittsburgh melanoma cohort Table 2). Baseline fecal microbiome composition was significantly different in patients with high and low NLR (FIG. 3a, p=0.024). The taxa significantly associated with high NLR included a high proportion of gram-negative organisms and were predominantly more abundant in Ps (FIG. 9).

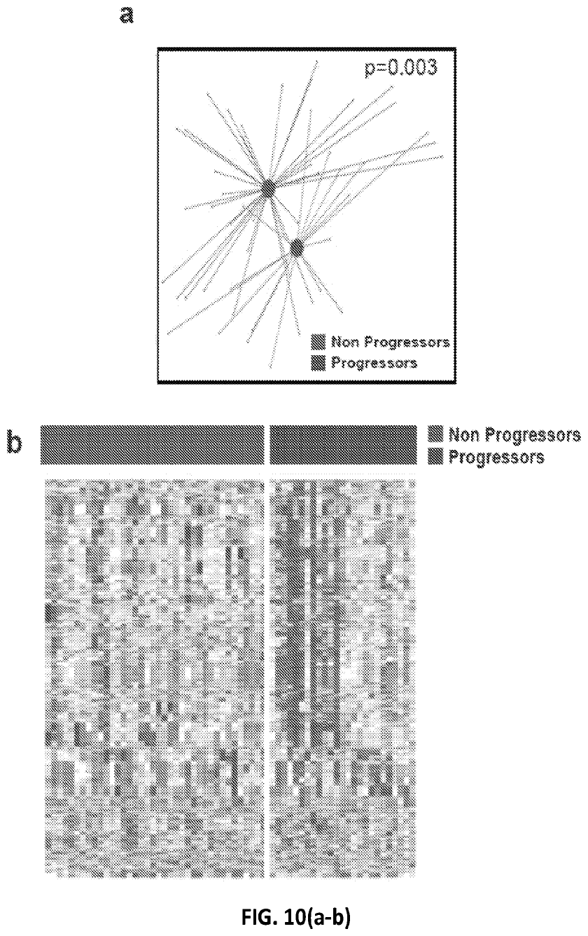

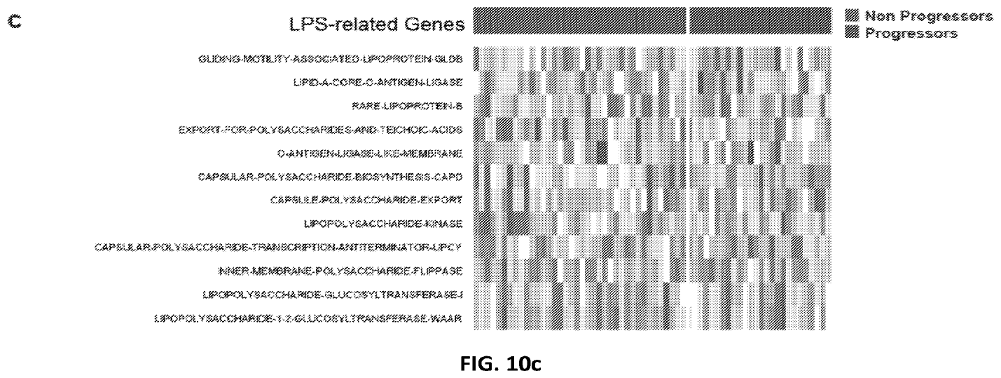

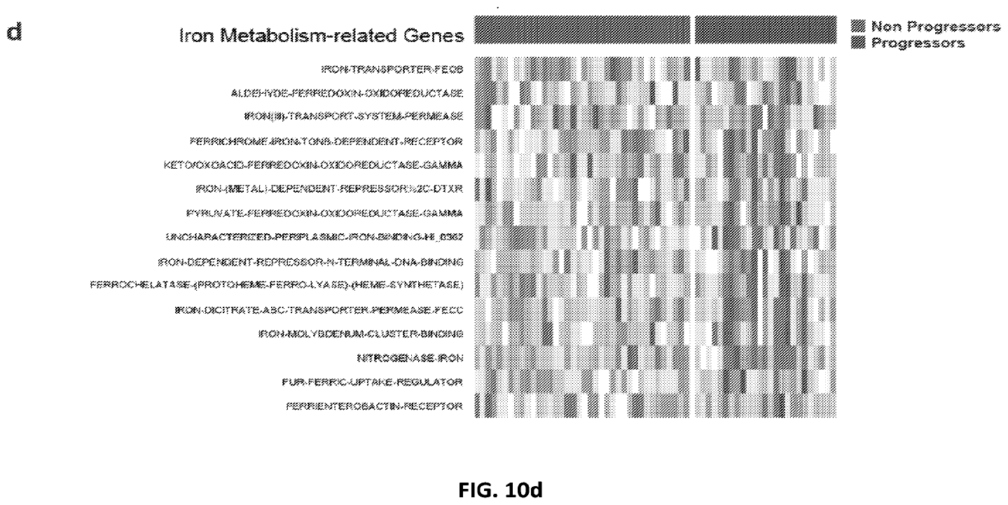

To delineate potential mechanisms used by the gut microbiota to influence patients' clinical outcome, we first performed analysis of microbial gene signatures of stool metagenomic data. Out of approximately 39,000 bacterial genes identified, 1,200 genes were differentially abundant between Ps and NPs [FDR<0.2 and fold change (FC)>1.5; FIGS. 10a and 10b]. Although most differentially abundant genes may reflect taxonomic rather than functional differences, we identified microbial signatures such as lipopolysaccharide (LPS) synthesis in Ps and iron bioavailability in NPs that may affect the host response (FIGS. 10b, 10c, and 10d). To evaluate the impact of this baseline intestinal microbiome composition upon host tissues, non-invasive transcriptomic analyses of cells shed into the intestinal lumen were performed using next generation RNA sequencing of stool samples as described previously (Knight, J. M., et al. 2014). The results show approximately 2000 host genes expressed at more than 10 reads per sample in 26 samples that were further compared between Ps and NPs. Genes encoding pro-inflammatory cytokines (IL1B and CXCL8), transcription factors (NFKB1Z, NFKB1A, TNFA1P3, and L1TAF), and superoxide dismutase (SOD2) were increased in Ps (FIG. 3b). NPs exhibited increased expression of genes encoding membrane associated mucins (MUC13, and MUC25) and apolipoproteins (APOA1, APOA4, and APOB) (FIG. 3b). Ingenuity pathway analysis (IPA) of upstream regulators of differentially expressed host genes identified LPS as a major contributor of pro-inflammatory gene signature identified in Ps (FIG. 3c). Gene set enrichment analysis (GSEA) analysis of predicted cell types based on genes identified above indicated that the most abundant cell type in stool samples from both Ps and NPs were intestinal epithelial cells (enterocytes and goblet cells). Fecal samples of Ps had also increased inflammatory cells (dendritic cells, monocytes, macrophages and neutrophils) (FIG. 3d).

To investigate regulatory relationships between host and microbes in PD-1 treated melanoma patients, we created a statistical model of robust interactions, so-called transkingdom network (Davar, D., et al. 2021; Rodrigues, R. R., et al., 2018; Yambartsev, A., 2016) between NLR, PFS, host gene expression and microbial taxa and genes, consisting of 684 nodes and 10,040 edges (FIG. 3e). Detrimental microbes had a greater influence upon PFS and NLR as illustrated by higher bipartite betweenness centrality (Bi-BC) scores (FIG. 3f, right panel) (Mann Whitney U p-value<0.05) between all microbes and human phenotypes (i.e., PFS and NLR). Average node degree did not differ significantly between beneficial and detrimental microbes (FIG. 3f, left panel). Collectively, our findings show that a detrimental gut microbiome enriched in gram-negative bacteria promotes an LPS-dominated inflammatory signature in the gut, resulting in systemic inflammation manifested by elevated NLR and poor response to anti-PD-1.

### Example 6

Intestinal Microbial Signatures are Associated with Distinct irAE Profiles in PD-1 Treated Melanoma Patients

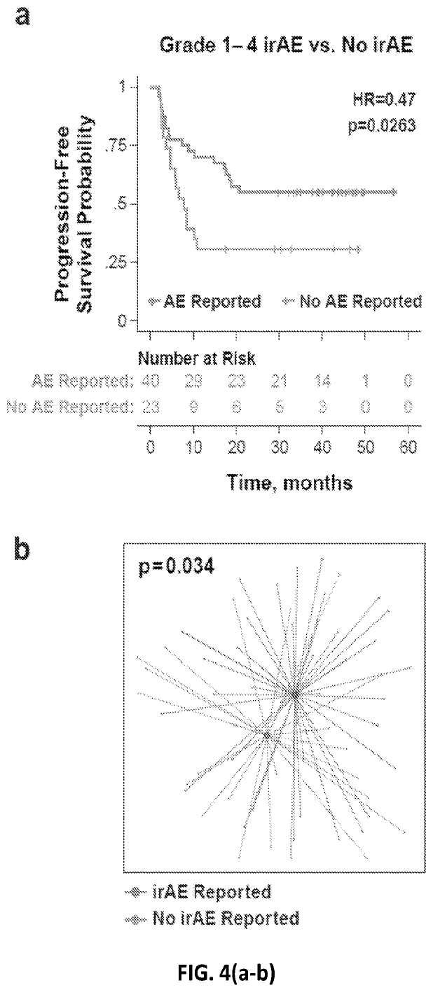

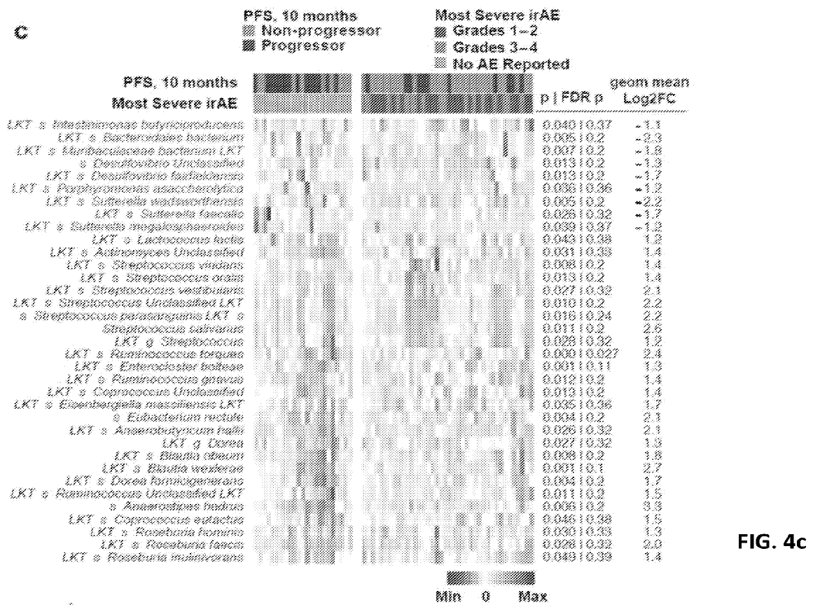

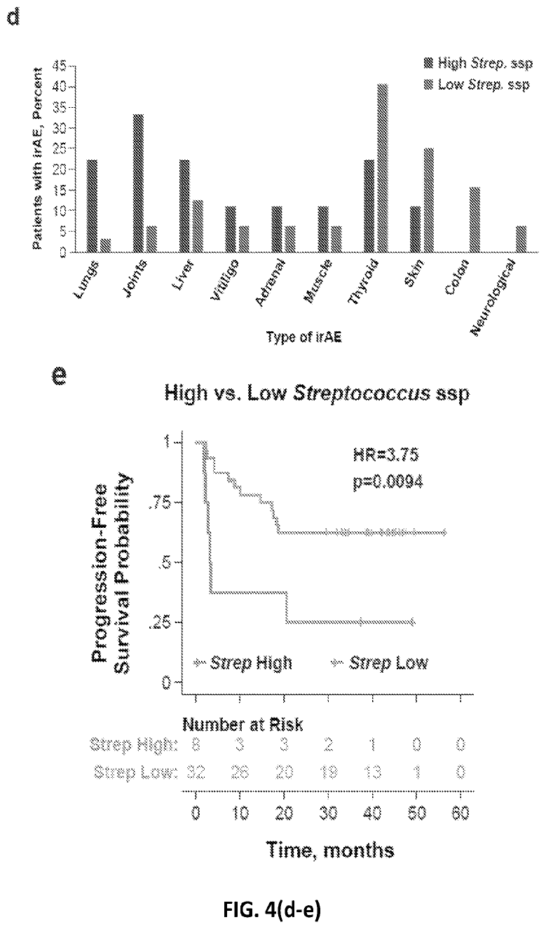

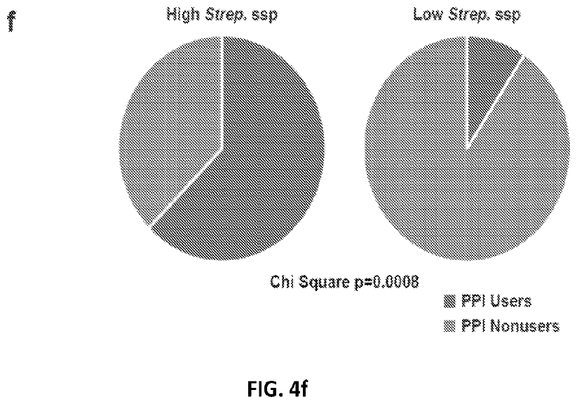

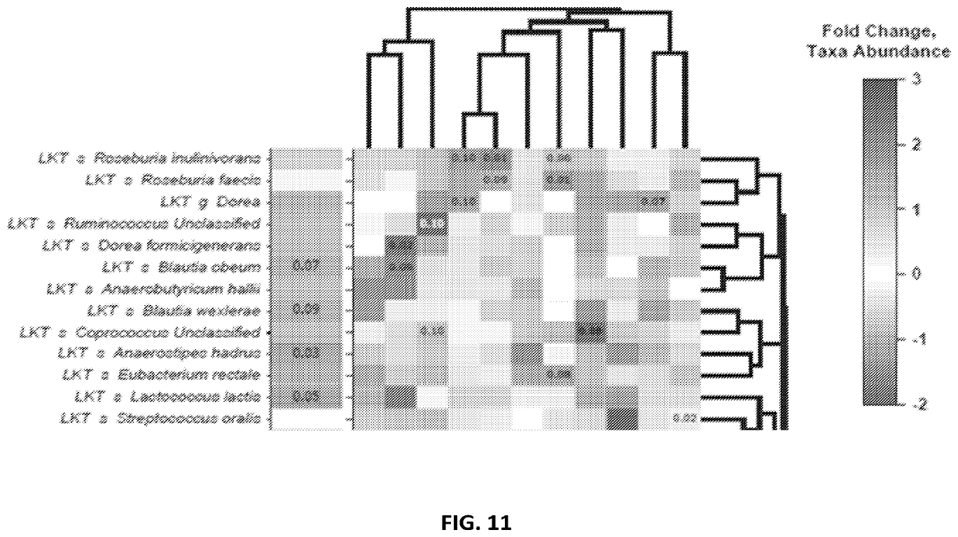

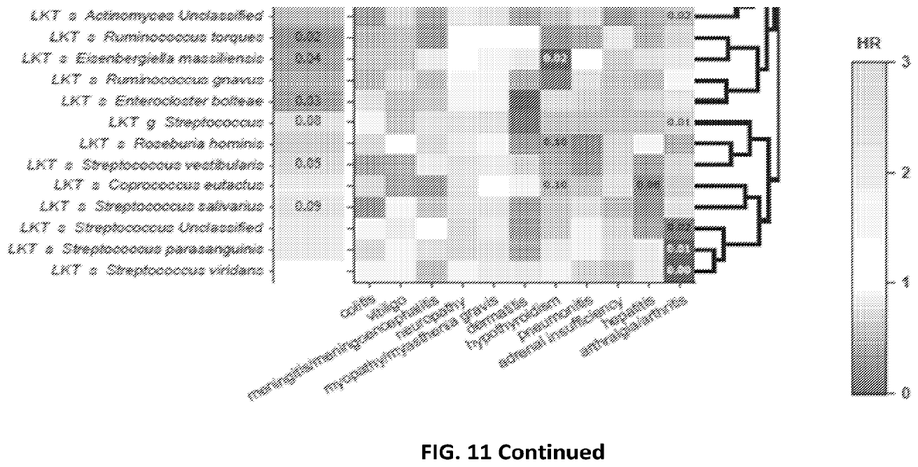

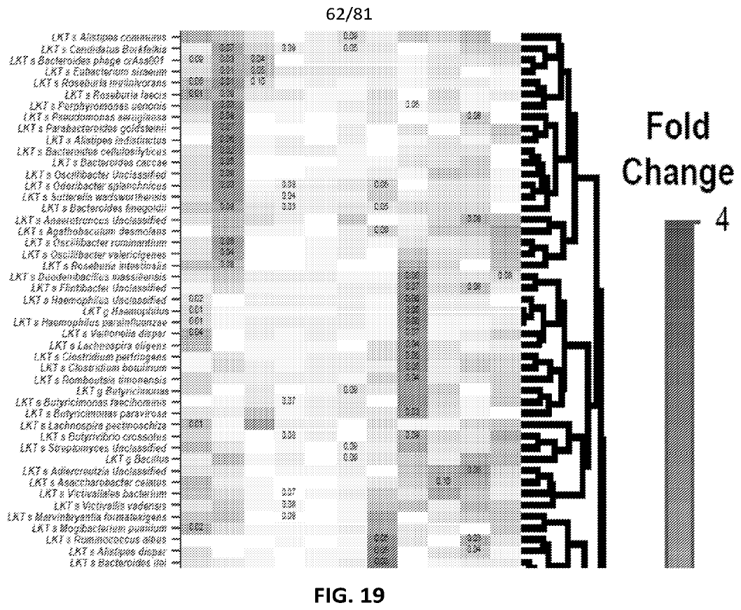

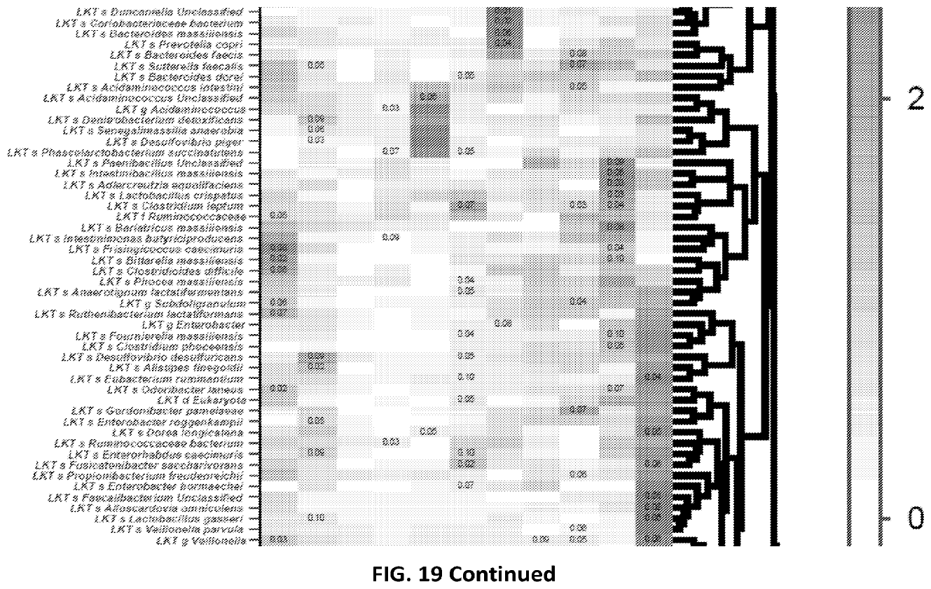

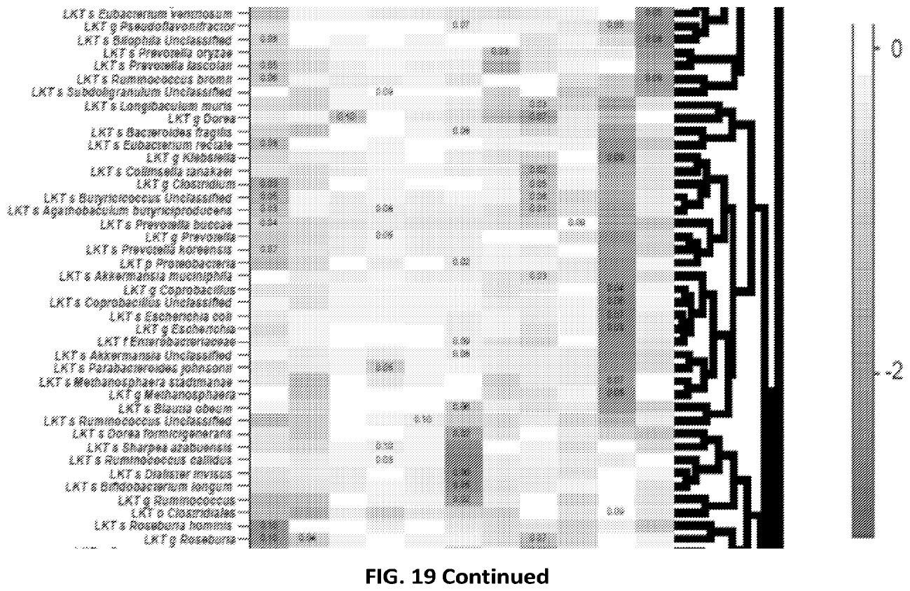

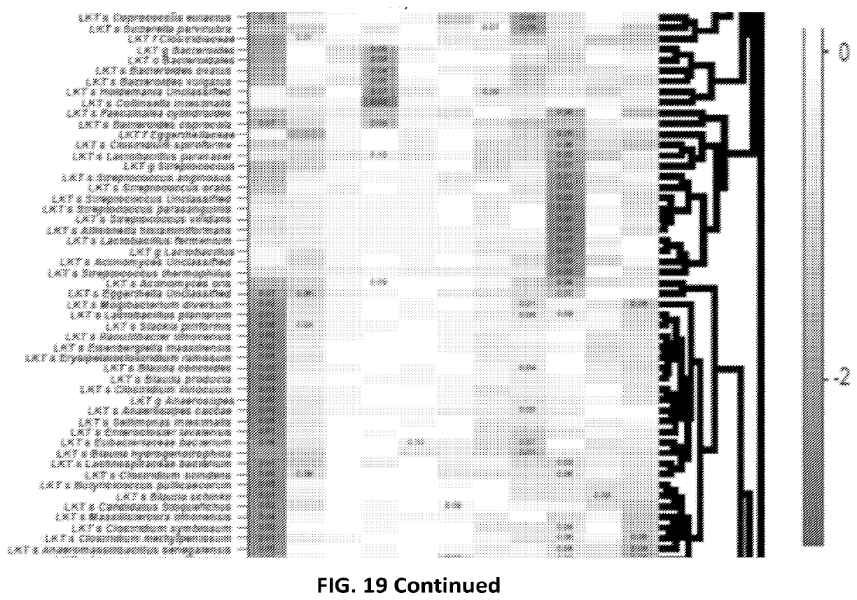

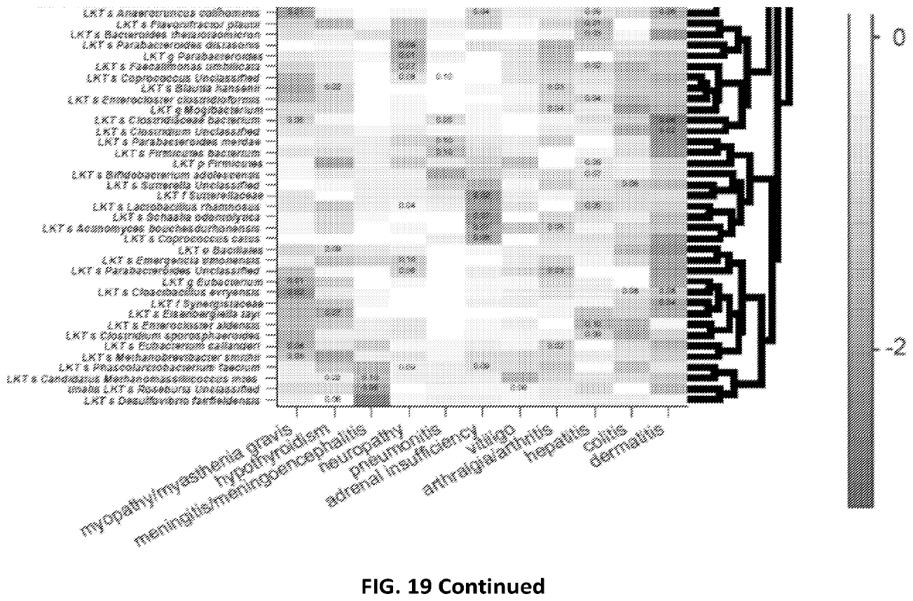

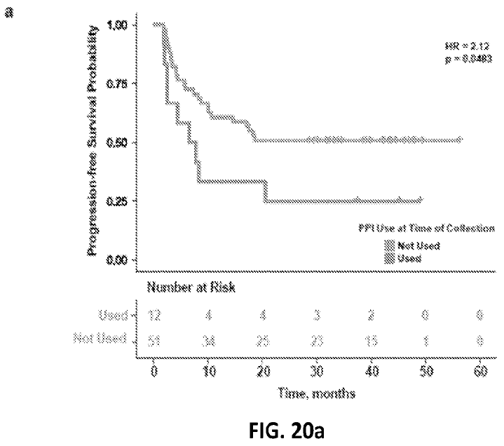

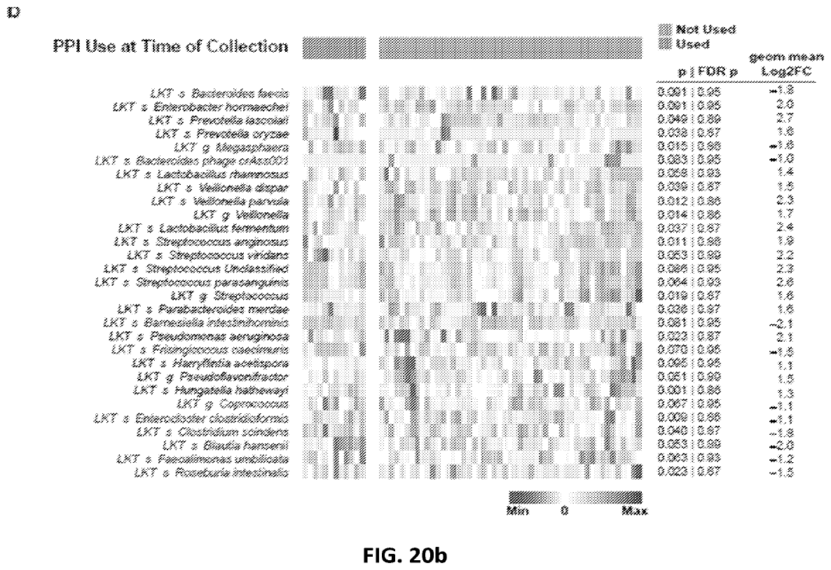

To determine the effect of intestinal microbiome composition on irAE occurrence, we analyzed data from patients in the early sample cohort. Concordant with previously published reports, any grade irAE was associated with improved PFS (Table 6 and FIG. 4a, p=0.0263) (Das, S., et al. 2020; Matsuoka, H., et al. 2020; Suo, A., et al. 2020). irAE occurrence was also significantly associated with baseline fecal microbiota composition (FIG. 4b, p=0.034). Two major bacterial taxa groups with opposite effects upon the anti-PD-1 response correlated with irAEs, including Lachnospiraceae spp. that mostly correlated with improved response and Streptococcus spp. that preferentially associated with poor response (FIG. 4c, FIG. 11). Patients developed distinct irAE profiles depending upon abundance of Streptococcus spp. in pre-treatment microbiome samples (FIG. 4d, FIG. 11). A cluster of 8 patients with the highest average abundance of the seven Streptococcus spp. that were significantly associated with irAEs (FIG. 4c) preferentially developed pulmonary, joint and hepatic manifestations; while patients with low Streptococcus spp. developed thyroid, cutaneous, gastrointestinal and neurologic irAEs (FIG. 4d). A detailed analysis of the individual bacterial species associated with the occurrence of individual irAEs uncovered specific microbial signatures linked to the occurrence of different types of irAEs (FIG. 19). These findings indicate that gut microbes regulate toxicity through distinct pathological mechanisms (FIG. 11, FIG. 19). Patients with lower Streptococcus spp abundance had significantly longer PFS compared to those with high Streptococcus spp. abundance (FIG. 4e, p=0.00935). The differential association of Streptococcus spp with various irAEs may account for the conflicting observations linking distinct irAEs with response across multiple published studies. High Streptococcus spp. abundance was correlated with PPI use during therapy (FIG. 4f, χ² p=0.0008), which in turn was associated with worse PFS (FIG. 20a, p=0.0483). PPI use was also correlated with the presence of intestinal bacteria associated with oralization, such as Streptococcus spp. and Veillonella spp. (FIG. 20b). Collectively, our findings have identified two distinct microbial signatures associated with two distinct irAE profiles in PD-1 treated melanoma. In line with prior reports, they also independently link PPI use with oralization (Horvath, A., et al. 2019), and adverse outcomes to anti-PD-147.

### Example 7

Meta-Analysis of Intestinal Microbial Sequencing Data in PD-1 Treated Melanoma Patients Identifies Compositional Differences Associated with Clinical Outcome

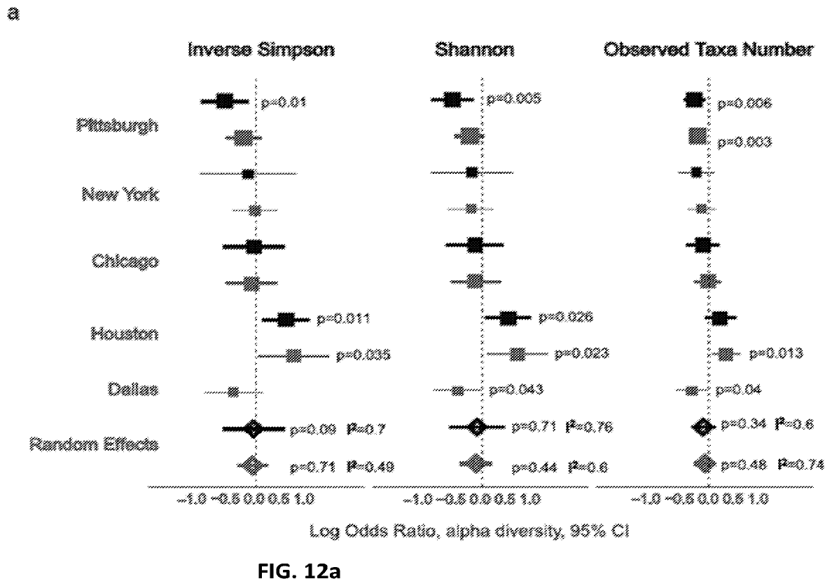

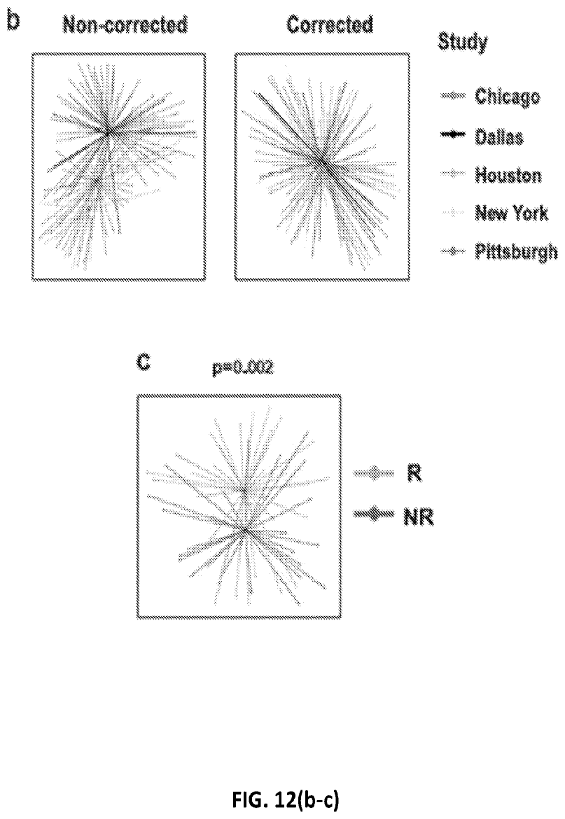

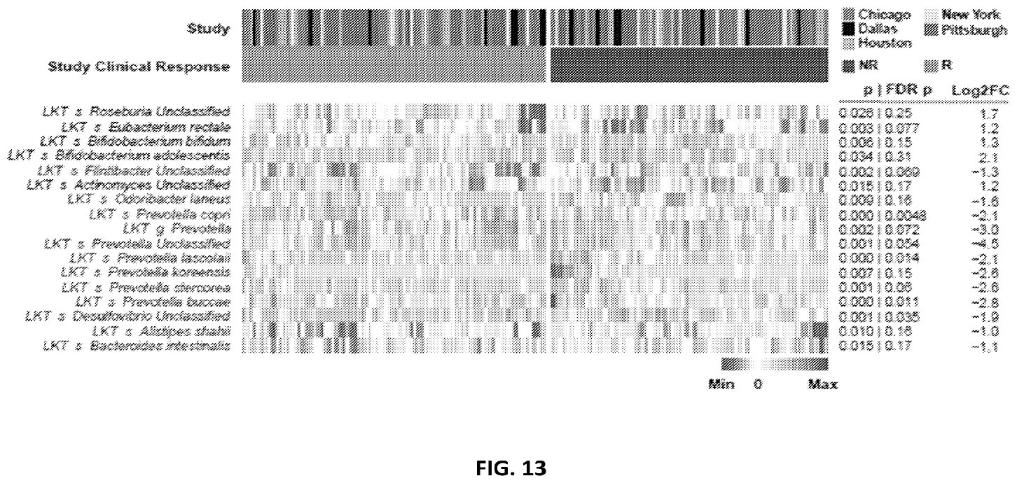

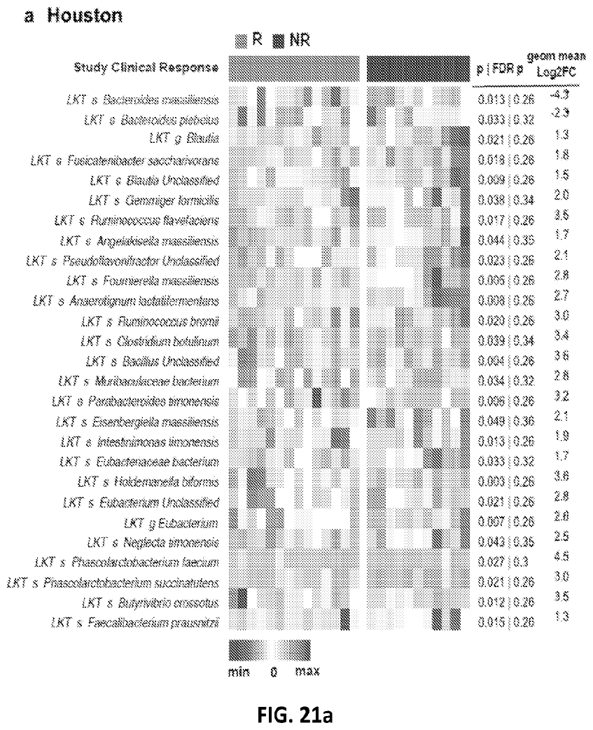

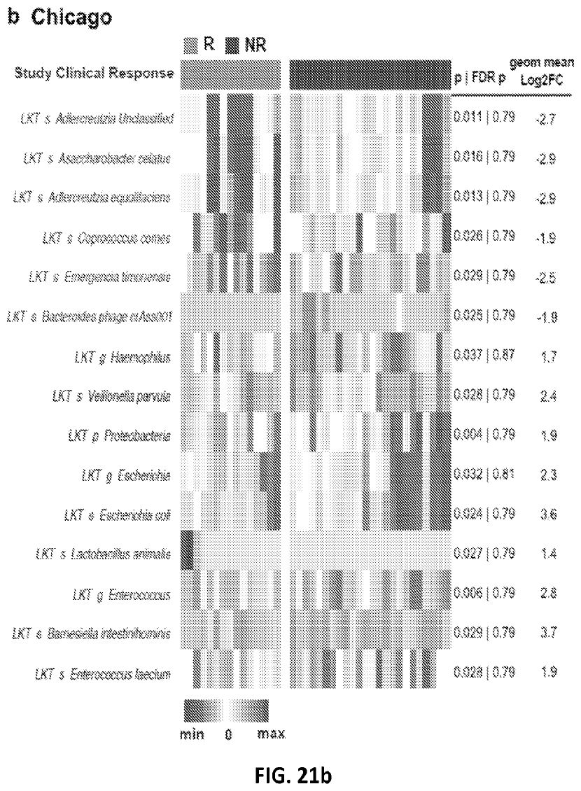

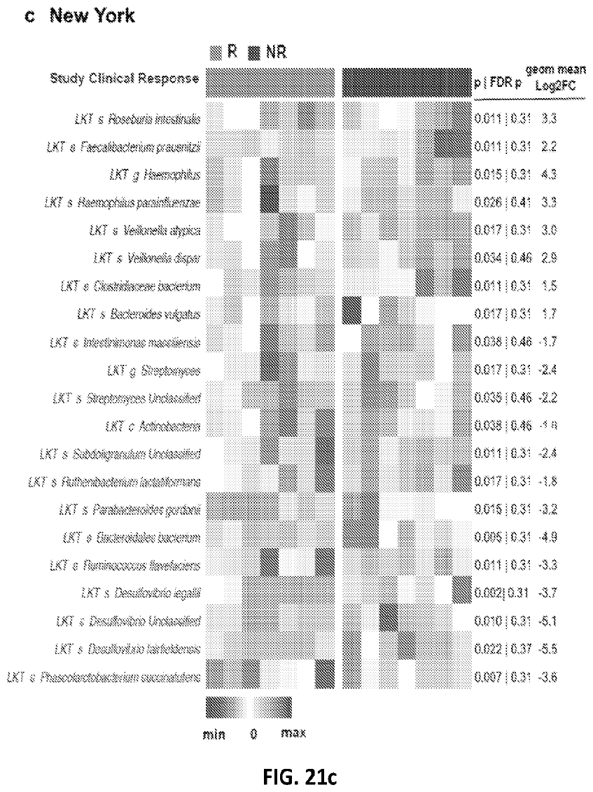

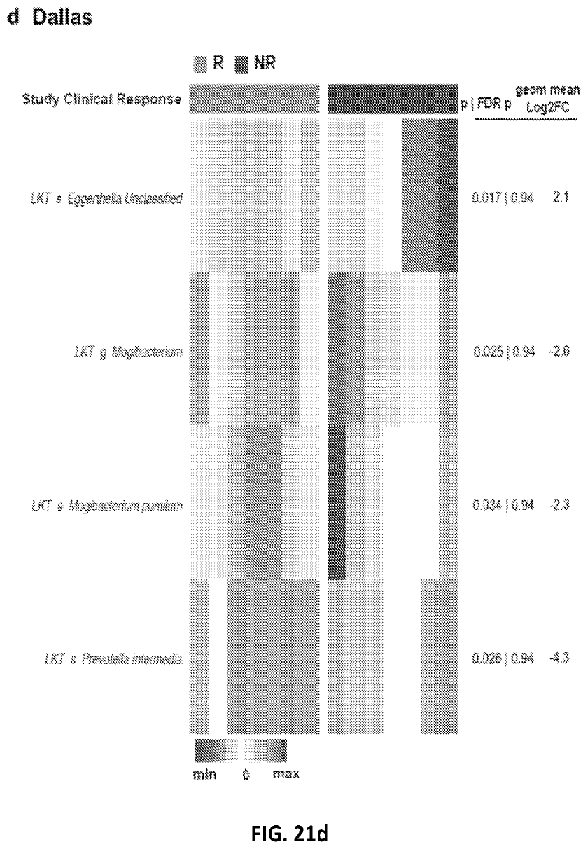

To reconcile disparities among published studies regarding the association of compositional differences and bacterial diversity of the gut microbiome with clinical outcomes to anti-PD-1 in melanoma patients, we performed a meta-analysis of four earlier published intestinal microbiota datasets of melanoma patients undergoing anti-PD-1 along with the Pittsburgh melanoma cohort (Table 3) (Gopalakrishnan, V., et al. 2018; Matson, V., et al. 2018; Frankel, A. E., et al. 2017; Peters, B. A., et al. 2019). The combined dataset comprised 155 shotgun and 150 16S amplicon sequenced samples. The definition of Rs and NRs patients was maintained as published in each individual study and summarized in Table 3. We observed that the alpha diversity in Rs compared to NRs for both shotgun (red, five cohorts) and 16S amplicon (black, four cohorts) sequenced samples was either decreased (Pittsburgh, shotgun/16S; Dallas, shotgun), unchanged (New York/Chicago; shotgun/16S) or increased (Houston; shotgun/16S) as measured by inverse Simpson, Shannon and observed taxa methods (FIG. 12a). Meta-analysis using a random-effects model indicated that none of the alpha diversity metrics were significant. The high 12 statistic (heterogeneity p-value<0.05) indicated that the studies were highly heterogenous. Reanalysis of the datasets using the same bioinformatics pipeline (Davar, D., et al. 2021; Stacy, A., et al. 2021; Kelsey, C. M., et al. 2021), revealed that only one of the four melanoma cohorts exhibited significant differences between Rs and NRs using PERMANOVA (Houston, p=0.011) (FIG. 12a). The evaluation of the different datasets mostly reproduced previously reported findings regarding taxonomic differences between Rs and NRs with minimal overlap between datasets (FIG. 21a-21d); however, we still observed significant inter-study heterogeneity (FIG. 12b, left panel). Batch correction using an empirical Bayesian framework implemented in ComBat R package (Benito, M., et al. 2004; Lazar, C., et al. 2013), strongly reduced this heterogeneity (FIG. 12b, right panel). The comparison of pooled batch effect corrected metagenomic data clearly distinguished Rs from NRs (FIG. 12c, p=0.002). The top taxa identified with nonparametric Mann-Whitney U test associated with Rs belonged to Actinobacteria phylum (Bifidobacterium bifidum, Bifidobacterium adolescentis and Actinomyces spp.) and Lachnospiraceae family (Roseburia unclassified, Eubacterium rectale) in the Firmicutes phylum (FIG. 13). Conversely, the top taxa associated with NRs belonged to either the Bacteroidetes (Prevotella spp., Bacteroides intestinalis, Alistipes shahii and Odoribacterium laneus) or the Proteobacteria (Desulfovibrio spp.) phyla (FIG. 13).

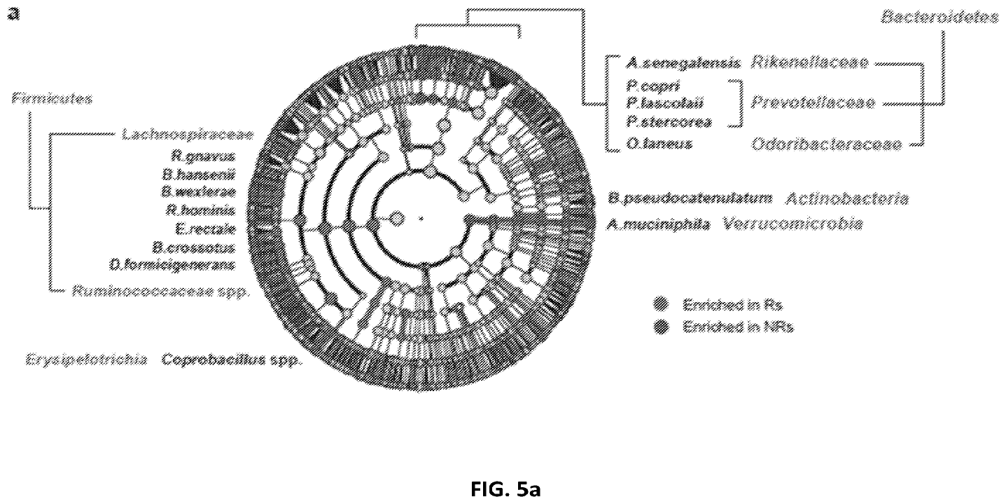

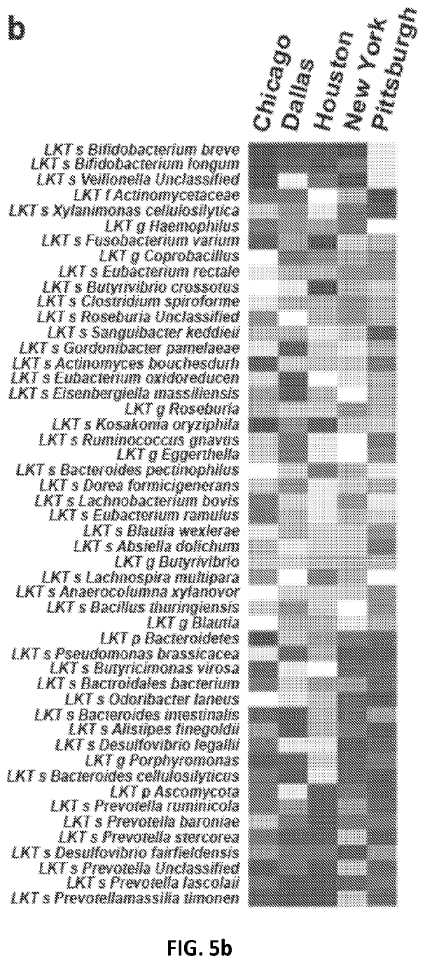

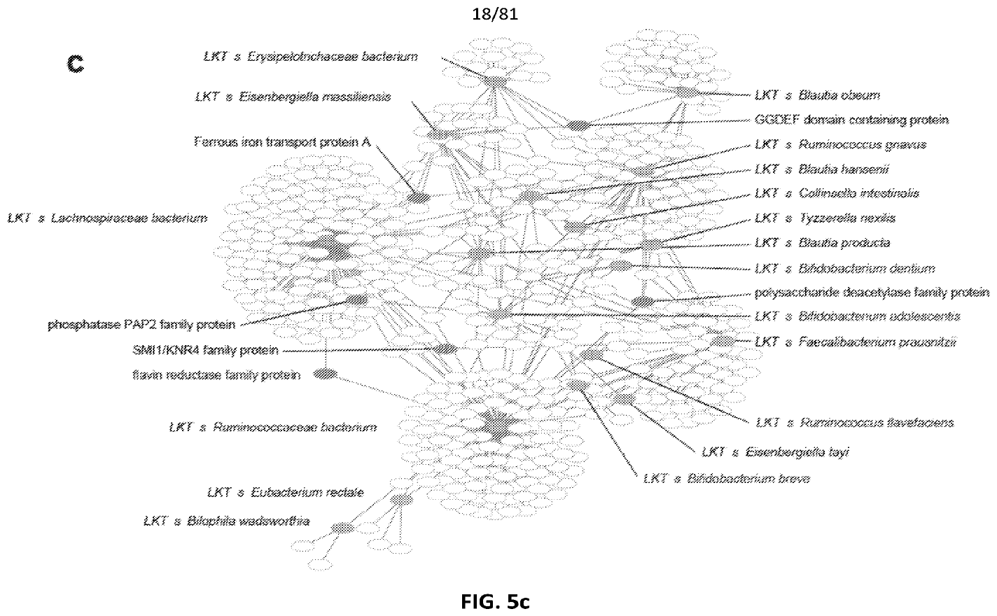

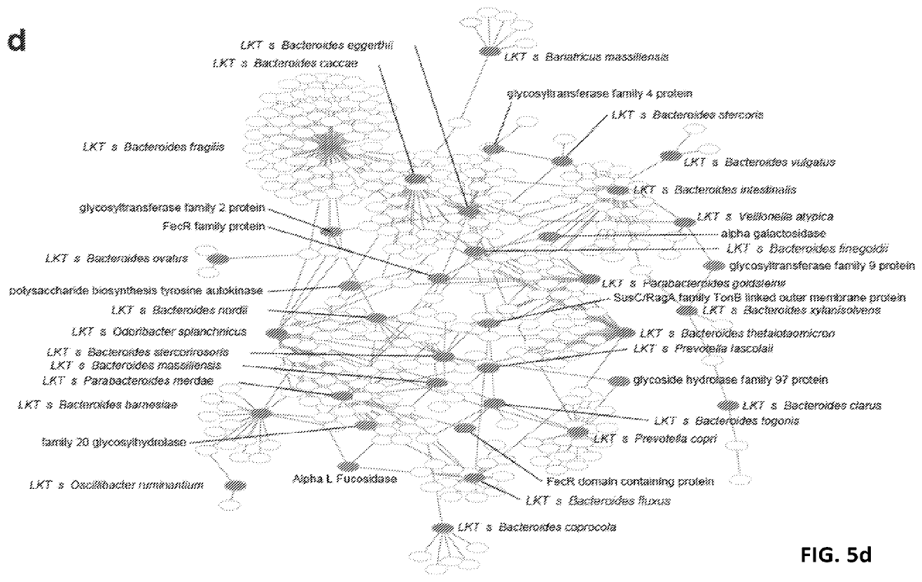

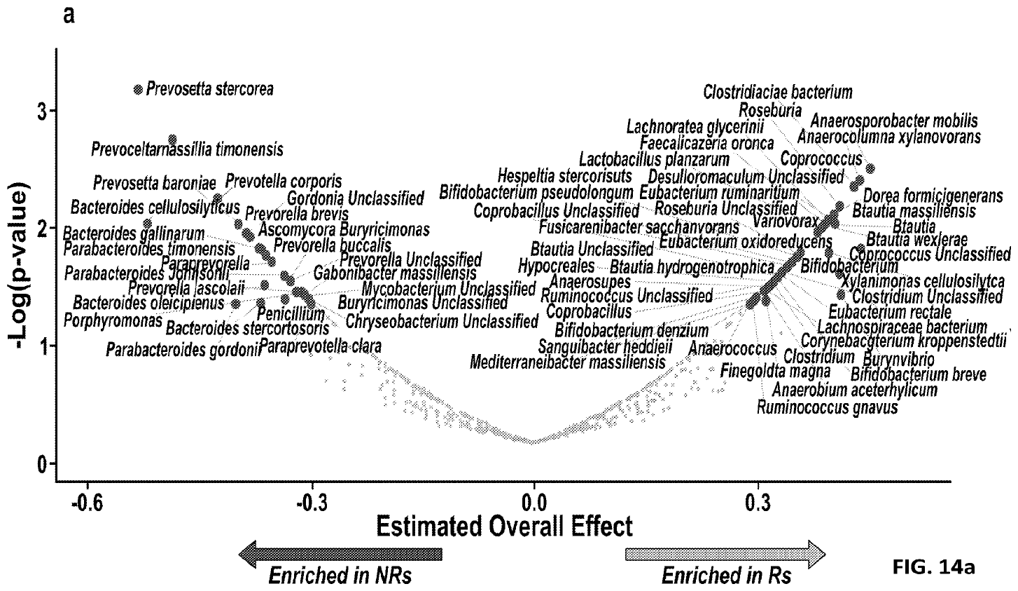

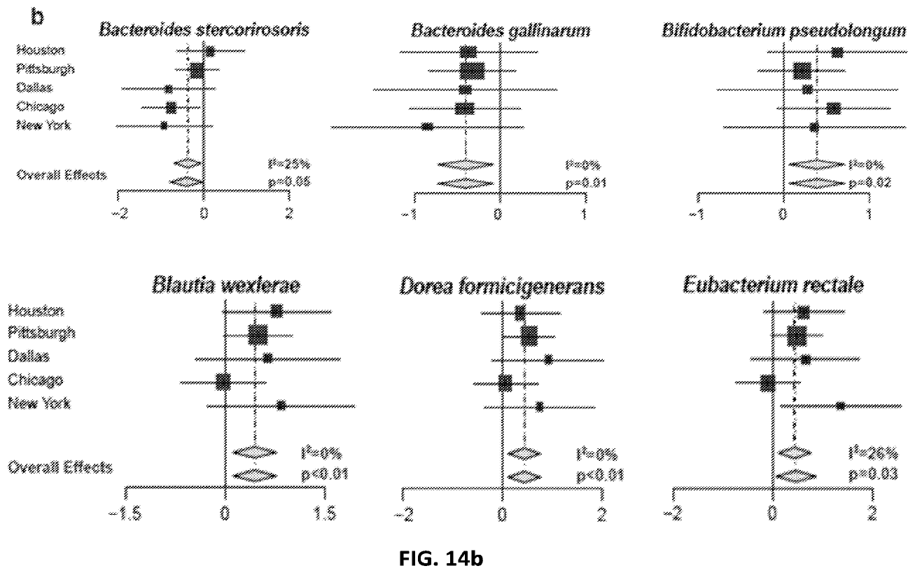

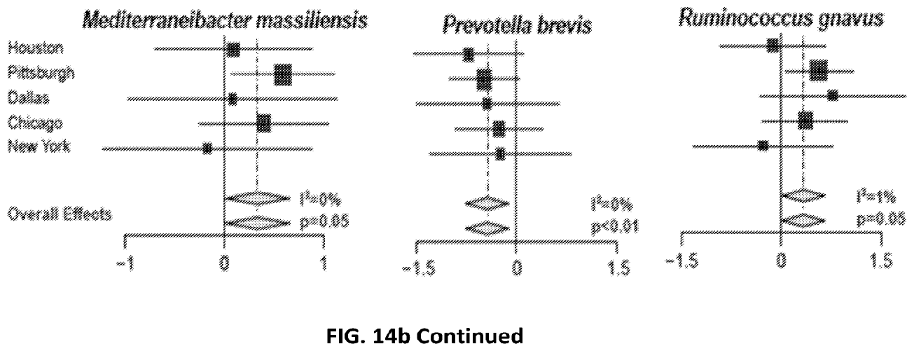

To dissect the contribution of different taxa at various taxonomic levels in determining response to anti-PD-1, we performed supervised comparisons of batch-corrected pooled microbiota data by linear discriminant analysis (LDA) effect size (LEfSe) analysis (Segata, N., et al. 2011). The analysis used the default logarithmic LDA score cutoff of 2.0 to identify taxonomic differences between Rs and NRs. The relative abundances of organisms belonging to Lachnospiraceae [Eubacterium rectale, Blautia spp., Ruminococcus (Mediterraneibacter) gnavus, Ruminococcus (Mediterraneibacter) torques, Eisenbergiella spp., Anaerostipes caccae, Sellimonas intestinalis] and unclassified species of the Erysipelotrichia class within Firmicutes phylum, and Actinobacteria (Bifidobacterium spp., Actinomyces spp., Schaalia odontolytica, Gordonibacter spp.) phylum were enriched in Rs compared to NRs (Table 7, LDA scores; FIG. 5a, LEfSe cladogram). Organisms selectively enriched in NRs primarily belonged to Prevotellaceae, Rikenellaceae, Porphyromonadaceae and Bacteroidaceae families within Bacteroidetes phylum, along with select members of Firmicutes (Lactobacillus spp.) and Proteobacteria (Desulfovibrio spp.) phyla. To evaluate whether these associations remained true in non-batch-corrected data, we performed two types of meta-analyses by combining study-level analyses between select bacteria in Rs and NRs from five cohorts (n=155) including the Pittsburgh early sample cohort (n=63) (Fisher's method, FIG. 5b; random effects model, FIG. 14). Across all studies, we observed that Bifidobacterium spp. Represented the taxa most significantly associated with clinical response (FIG. 14). In line with previous published findings (Gopalakrishnan, V., et al. 2018), Faecalibacterium spp. was also associated with positive outcome to anti-PD-1 while multiple Prevotella spp. within the Bacteroidetes phylum were enriched in NRs (FIG. 14).

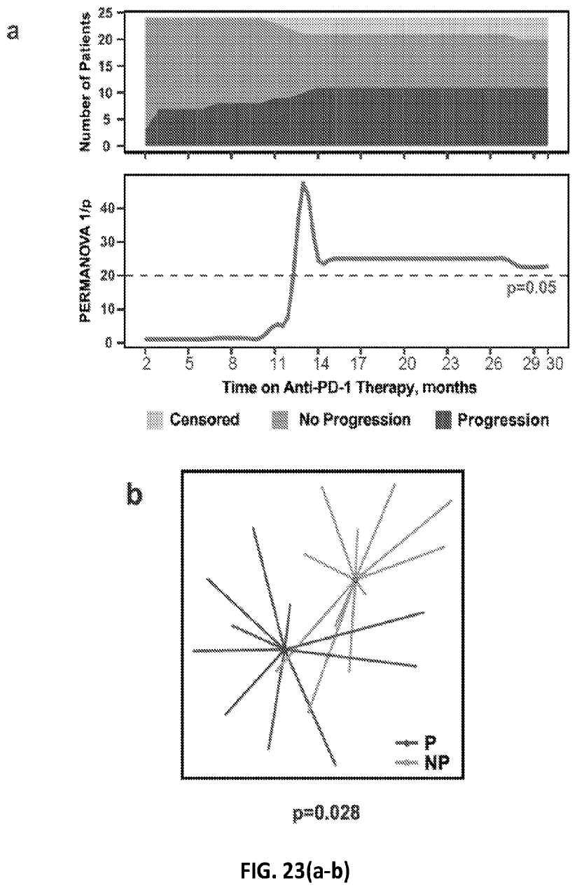

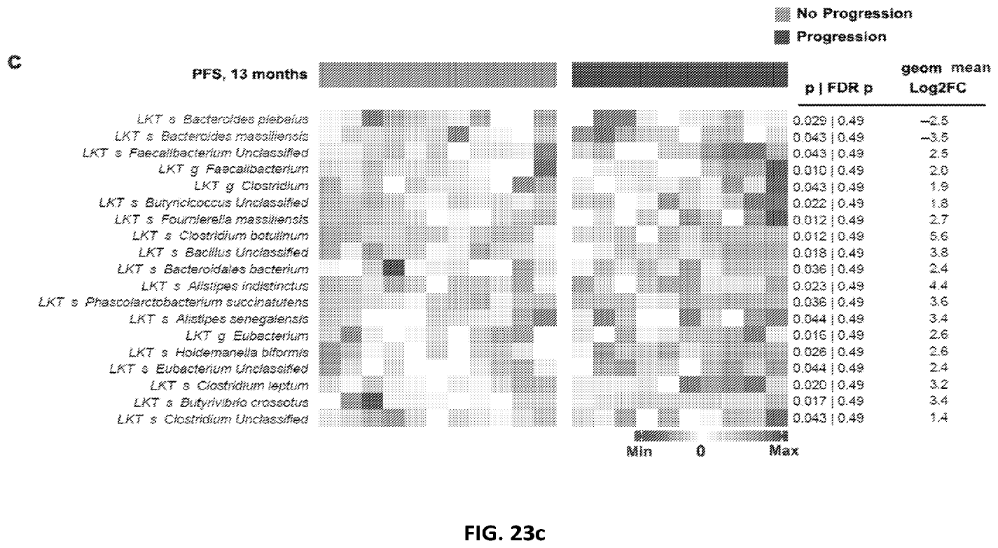

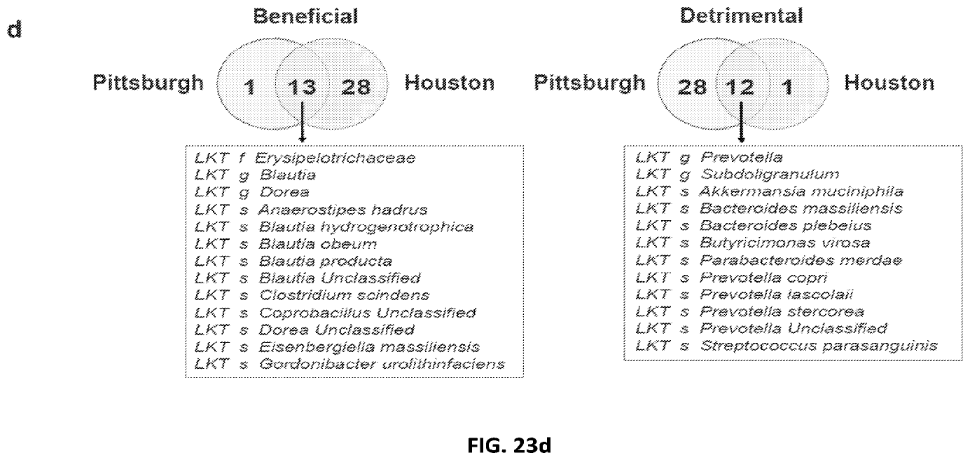

To independently validate the predictive power of gut microbiota composition upon PFS observed in the Pittsburgh cohort to anti-PD-1 in an independent PD-1 treated melanoma cohort (Houston) with available time-to-event information (Gopalakrishnan, V., et al. 2018). Like the Pittsburgh early sample cohort, baseline fecal microbiota composition as evaluated by shotgun sequencing showed maximal separation of Ps from NPs at 13 months (FIGS. 23a and 23b). At this timepoint, we observed microbial signatures that resembled other datasets, including increased abundances of Bacteroides massilliensis in Ps, and Faecalibacterium spp. and Eubacterium spp. in NPs (FIG. 23c). As previously reported for the early sample cohort, we also performed a separate analysis wherein we evaluated the beneficial or detrimental impact of bacterial species by determining fecal abundance cut points for all organisms and calculating hazard ratios (HR) in relation to PFS using Evaluate Cutpoints (Ogluszka, M., 2019). This approach identified the same taxa identified by Mann-Whitney U test (FIG. 23c), along with many additional taxa associated with either improved or decreased PFS (Tables 8 and 9). The taxa significantly associated with either improved or decreased PFS in both the Pittsburgh early sample and Houston cohorts comprise many of the species identified in several studies as modulators of clinical outcome to anti-PD-1 (FIG. 23d).

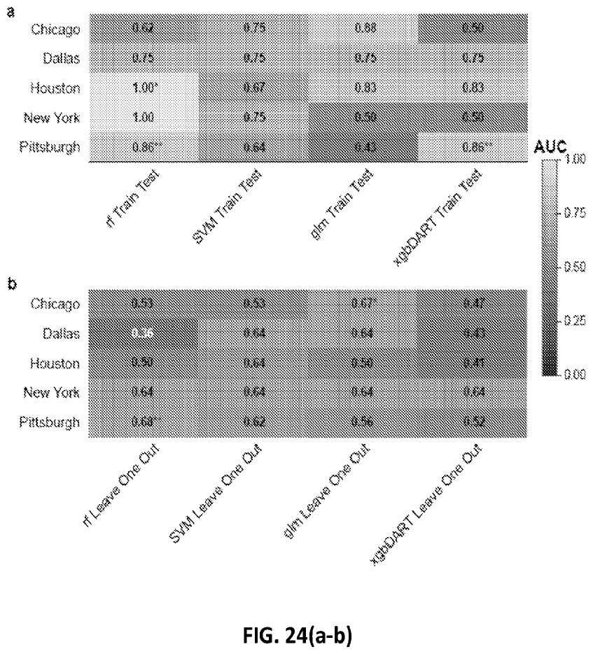

To use features of existing metagenomic datasets to detect hard-to-discern patterns, several machine learning models were utilized to predict clinical outcome. four separate algorithms were independently evaluated using one of two validation techniques: 70:30 train-test splitting, wherein 70% of the particular dataset was used to train and 30% was used to test the model (FIG. 24a); and leave-one-out validation, wherein the model was trained on the combined dataset excluding a particular cohort and tested on the particular cohort (FIG. 24b) using area-under-curve (AUC) as a scale-invariant metric of classifier performance. Using train-test splitting, it was observed that most AUC values were non-random (>0.50) although smaller cohorts (New York and Dallas) did not achieve statistical significance while larger cohorts (Pittsburgh and Houston) did (FIG. 24a). This study also evaluated the generalizability of microbial signatures across all cohorts using leave-one-out validation (FIG. 24b). Here, the overall performance of all models dropped was observed although Pittsburgh cohort performed significantly better than chance. The reduction in predictive power despite the increase in cohort size suggested the presence of an unknown factor that would need to be identified to improve the performance of the model.

### Example 8

### Beneficial and Detrimental Intestinal Taxa Exhibit Distinct Functional Gene Signatures

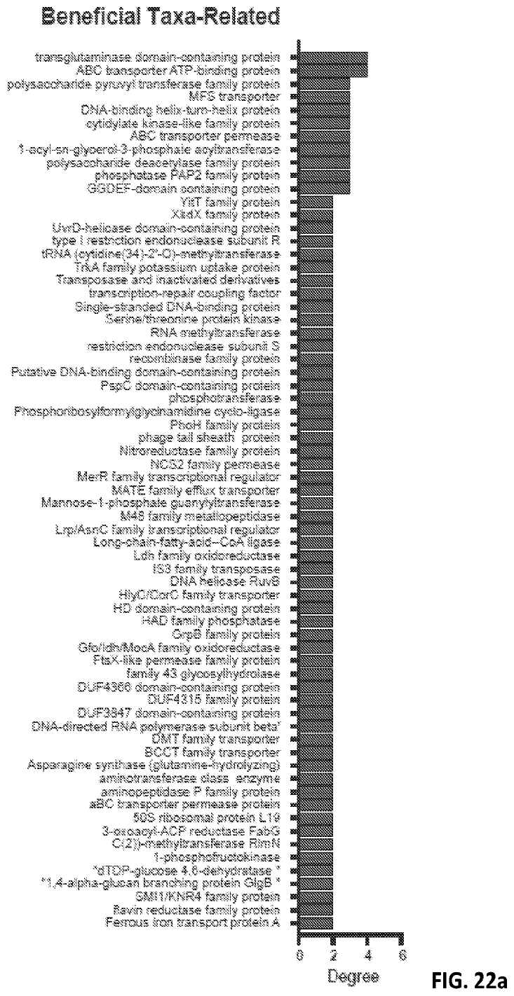

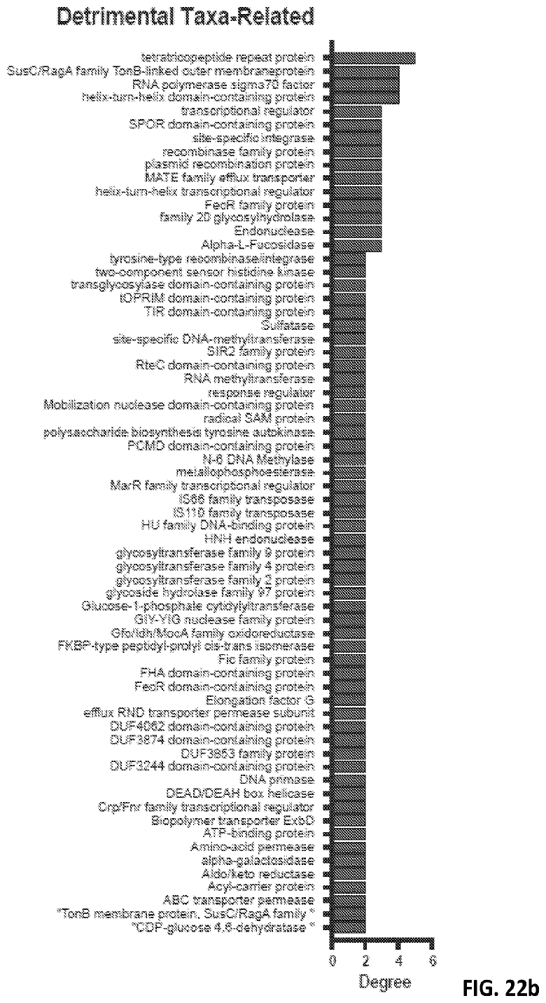

As noted above, differentially expressed microbial genes in Rs compared to NRs are expected to mostly reflect taxonomic differences, resulting in the identification of genes specific for entire taxa (passenger genes) rather than genes that directly impacted host response status (driver genes). Notably, we identified similar gene signatures associated with host response across different taxa, suggesting that unknown variance in complex microbial communities precluded robust signature identification. Therefore, to comprehensively identify microbial signatures, we performed a reference-independent binning analysis of the assembled contigs from the five melanoma patient cohorts similar to MetaBAT approach (Kang, D. D., 2015; Kang, D. D., et al. 2019). 13,237 different clusters were found that belonged to 730 different taxonomic units and identified 39,480 genes. This approach permitted strain-level gene composition comparisons, and the nonparametric statistical comparisons are presented as species-gene networks of beneficial (FIG. 5c) and detrimental (FIG. 5d) taxa, along with the “degree” statistics (number of taxa shared by a gene) (FIG. 22). Within beneficial taxa, we identified increased abundance of genes related to polysaccharide deacetylation known to play a role in immune evasion (Balomenou, S., et al. 2013) as well as iron transport and iron-induced production of reactive oxygen species (ROS), which affect mucosal healing (Bessman, N. J., et al. 2020). Increased abundance of genes encoding flavin and riboflavin metabolism was observed, byproducts of which can be presented by MR1, a non-polymorphic major histocompatibility complex class I-related molecule that is recognized by mucosal-associated invariant T (MAIT) cells and possibly MR1-restricted T cells (Crowther, M. D., et al. 2020; Salio, M., et al. 2020). Conversely, within detrimental taxa, this study identified increased expression of genes related to LPS synthesis and mucus degradation such as alpha-L-fucosidase (Fan, S., et al. 2016) and alpha-galactosidase (Wright, D. P., 2000). Collectively, the findings herein indicate that beneficial and detrimental intestinal taxa in PD-1 treated melanoma exhibit distinct species-gene network signatures.

### Example 9

### Phylogenetic Profile Similarities Identify Beneficial and Detrimental Enteric Microbiotypes in PD-1 Treated Melanoma Patients

Several lines of evidence indicate that human gut microbiome composition includes multiple discrete ecologically balanced communities—hereafter referred to as “enteric microbiotypes”—that tend to be resilient although still modifiable by diet, drugs and lifestyle (Arumugam, M., et al. 2011; Brooks, A. W., 2018; Dwiyanto, J., et al. 2021; Gorvitovskaia, A., 2016; Knights, D., et al. 2014; Zhang, R., 2021). While much work has linked composition of individual taxa to outcome to anti-PD-1, little is known about the relationship between distinct enteric microbiotypes and response to anti-PD-1.

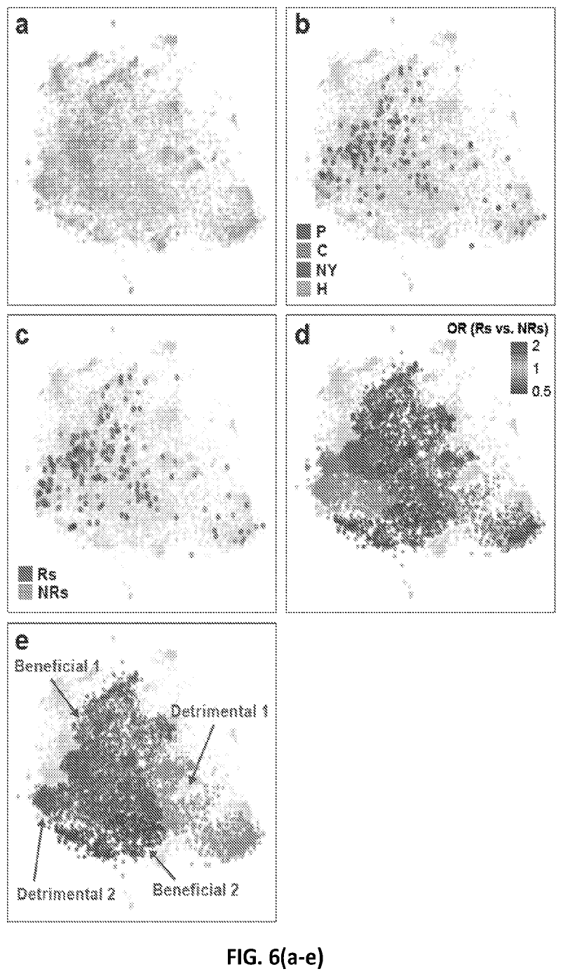

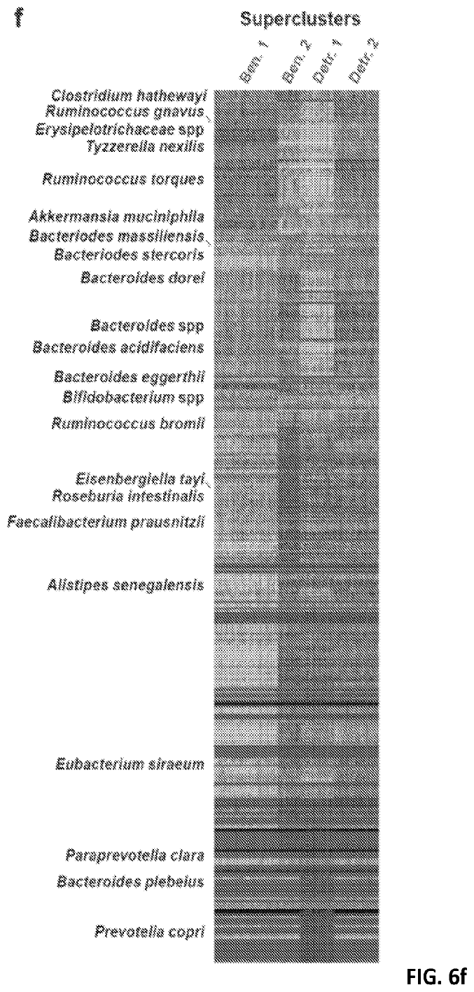

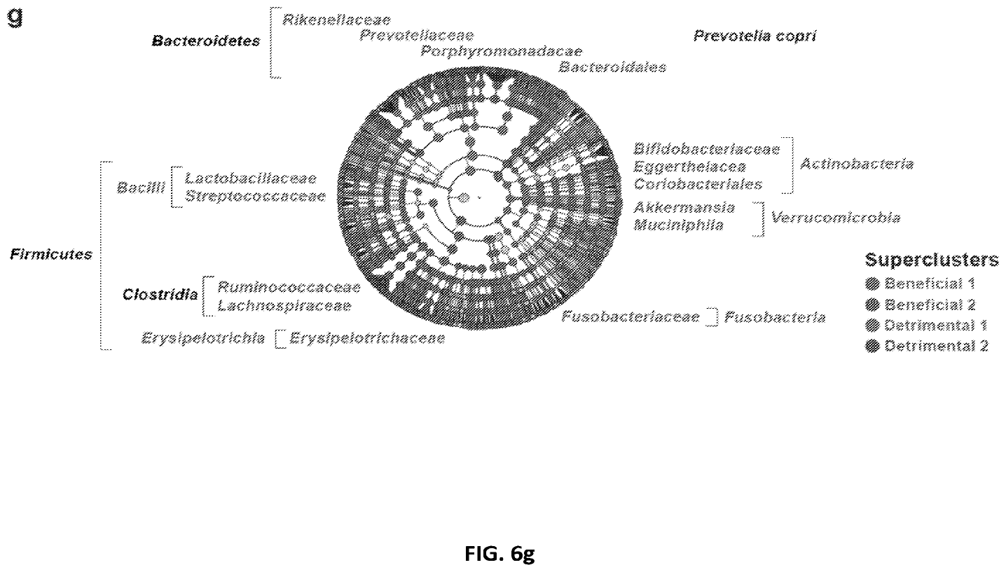

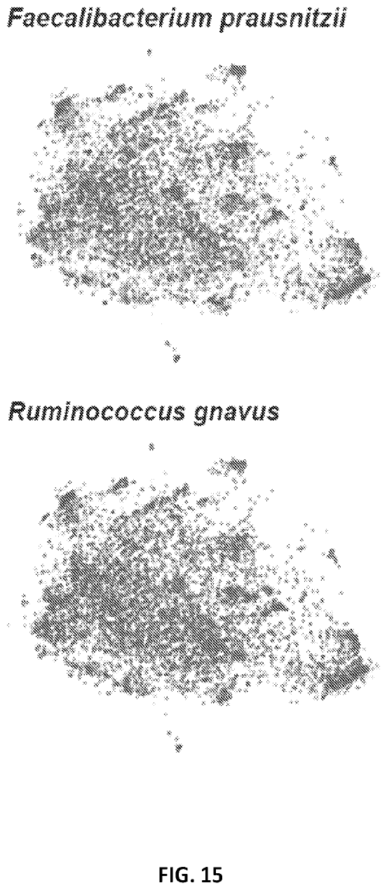

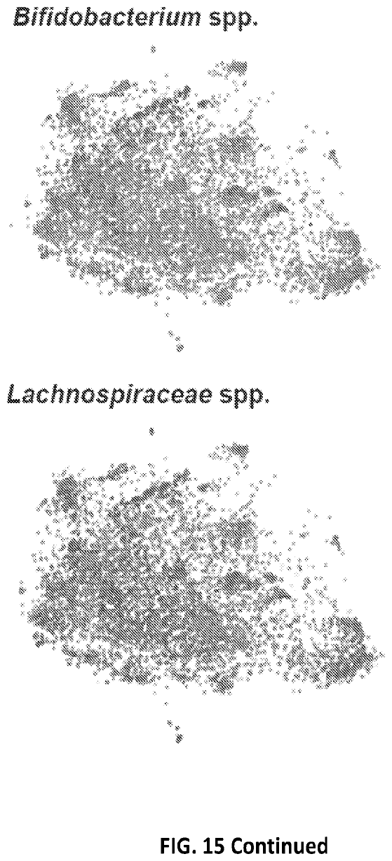

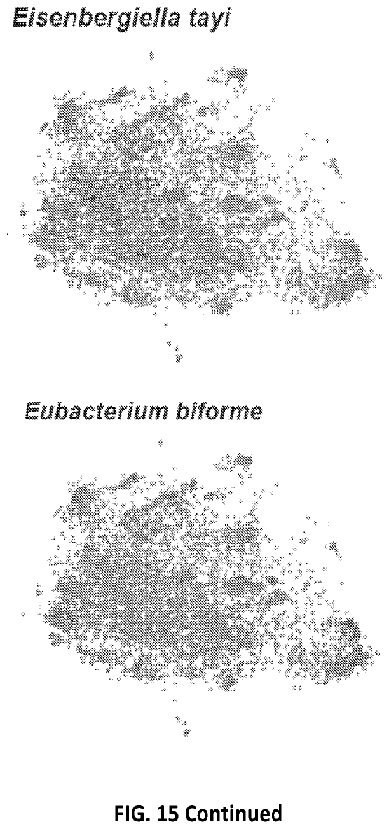

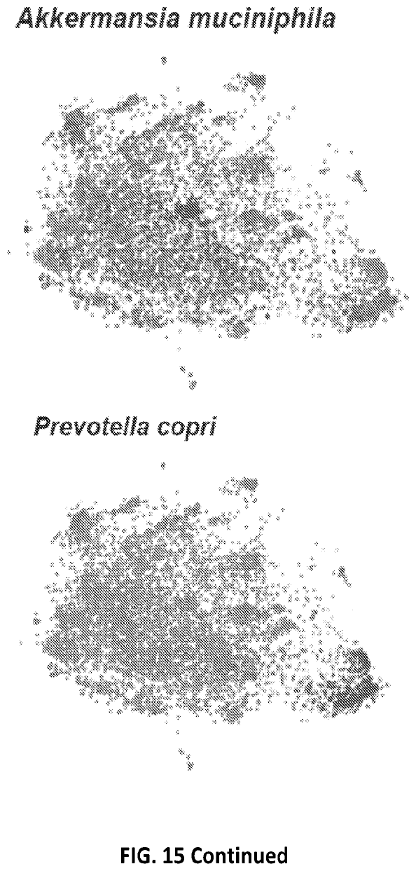

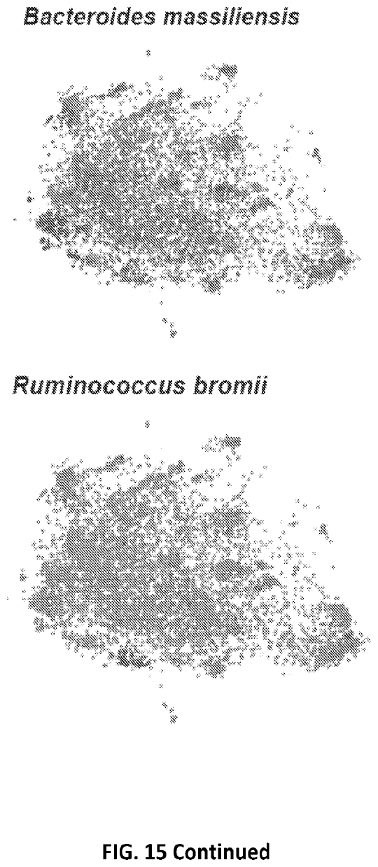

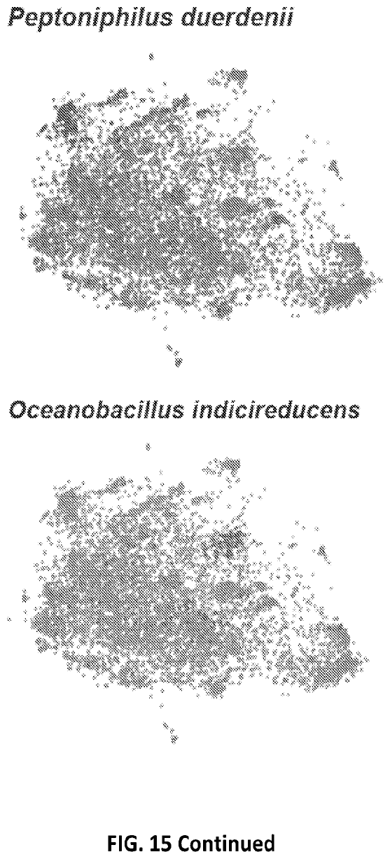

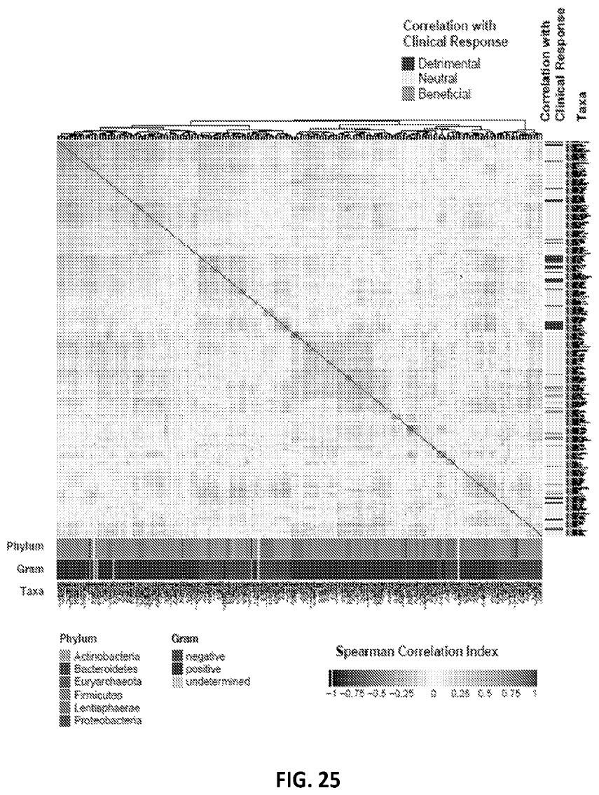

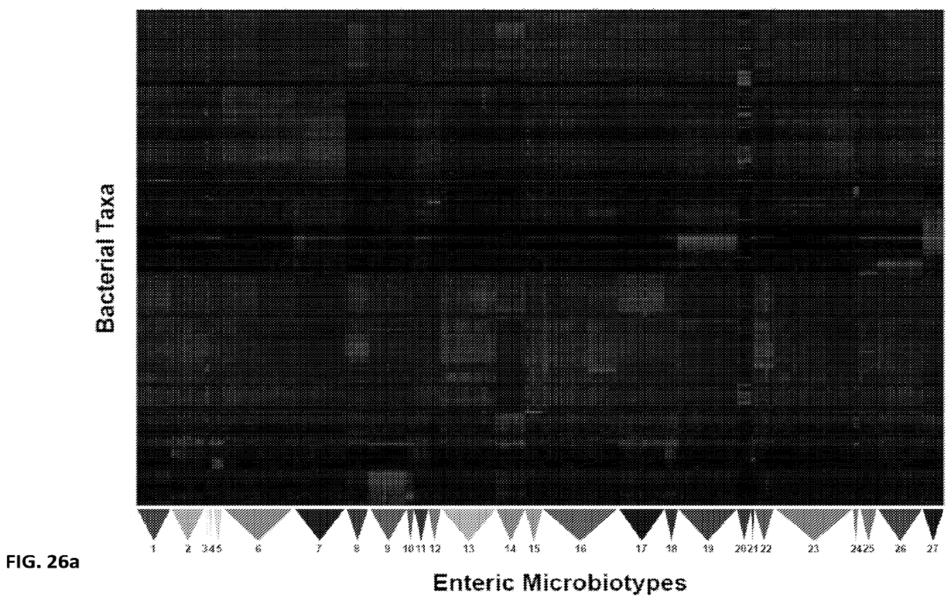

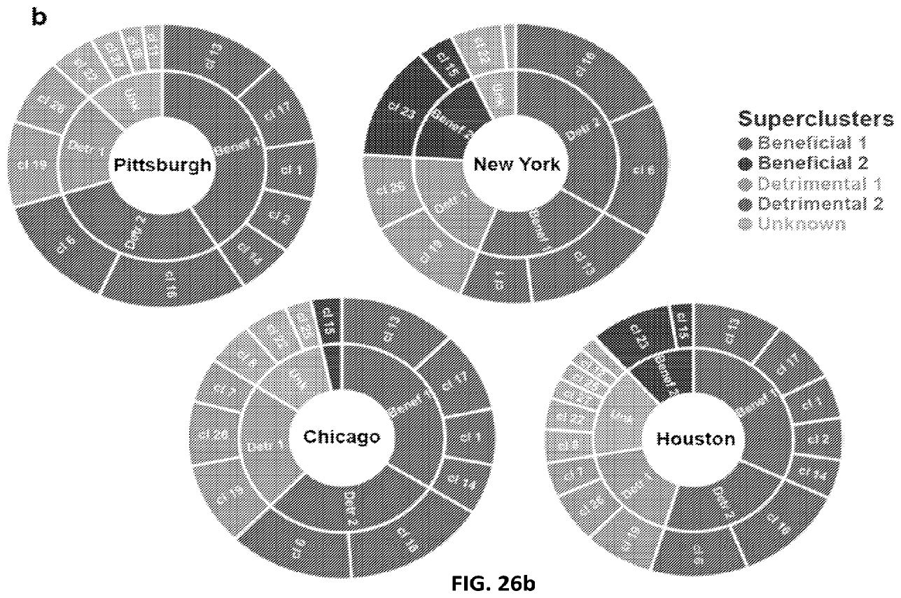

Investigating the inter-taxa relationships using a correlation matrix of metagenomic data from all cohorts, we observed that beneficial and detrimental bacteria identified in the five melanoma cohorts segregated in several distinct clusters (FIG. 25). To better define these clusters, this study uniformly analyzed 16S rRNA gene amplicon data from PD-1 treated melanoma patients available from four independent melanoma cohorts and the American Gut Project (AGP, FIG. 6a) (McDonald, D., et al. 2018). Using Phenograph clustering software, 27 distinct microbiotypes were identified (FIG. 6a). Some of these microbiotypes were quite distinct (e.g. clusters 9 and 19), while others (e.g. clusters 13 and 27) had overlapping features with neighboring clusters (FIG. 26a) (Levine, J. H., et al. 2015). Concordant with initial reports of enterotypes in human gut microbiome, we identified many enteric microbiotype-defining taxa including previously reported Prevotella spp. Along with other organisms including Akkermansia spp., Bifidobacterium spp. and others (FIG. 15). Mapping 16S amplicon data (tSNE plots) from PD-1 treated melanoma patients to references sequences obtained from the AGP shows that each patient of the four cohorts was differentially distributed within the AGP map (FIG. 6b, FIG. 16a). By mapping melanoma patients onto AGP tSNE by response status, we observed that Rs and NRs were distributed unequally across the AGP map (FIG. 6c). However, by calculating odds ratios of Rs to NRs in each cluster, we observed that Rs and NRs segregated into compositionally-distinct beneficial (blue) and detrimental (red) enteric microbiotypes that had differential probabilities of response (FIG. 6d). This study identified four superclusters that broadly grouped together microbiotypes with similar correlation between microbial composition and clinical response: two were enriched in beneficial taxa and two others were enriched in detrimental taxa (FIG. 6e). ANOVA (FIG. 6f) and LDA plot (FIG. 6g and Table 10) were used to evaluate the most differentially abundant taxa within each supercluster. Beneficial superclusters included Faecalibacterium prausnitzii and Ruminococcus bromii (beneficial 1), as well as Bifidobacterium spp., Streptococcus spp., Erysipelatoclostridium spp., Blautia wexlerae, Ruminococcus (Mediterraneibacter) gnavus, Ruminococcus (Mediterraneibacter) torques, Tyzzerella nexilis, and Eisenbergiella tayi (beneficial 2). Conversely, detrimental microbiotypes included Bacteroides massiliensis, Bacteroides stercoris (detrimental 1) as well as Prevotella copri, and Bacteroides plebeius (detrimental 2) (FIG. 6F, 6G and Table 10). Taken together, these findings indicate that distinct enteric microbiotypes comprising taxonomically unrelated organisms in melanoma patients are associated with differential probability of response to anti-PD-1.

Given the significant differences in microbial taxa abundances observed in the multiple cohorts from different cities within the United States and well-established contribution of host location to microbiota variation (Gopalakrishnan, V., et al. 2018; Matson, V., et al. 2018; Frankel, A. E., et al. 2017; Peters, B. A., et al. 2019; He, Y., et al. 2018 we sought to define the effect of geography upon enteric microbiotype distribution. Using geolocation data associated with each individual stool sample in AGP cohort, every sample was mapped and proportional distribution of each cluster at the county-level was calculated using three individuals per county per cluster as a cutoff. The data was further scaled by number of individuals per state and per cluster and is depicted in relation to the 28 states that met the cutoff and, separately on the right, in relation to the 4 states from which the 4 studied cohorts originated (FIGS. 16b and 16c). Concordant with a prior report in different districts in Southern China (He, Y., et al. 2018), preponderances of distinct clusters were observed at the US state level (examples of 4 representative microbiotypes with uneven geographical distribution are shown in FIG. 16d)—another possible mean by which inter-study differences could be reconciled. The distribution of the microbiotypes and supercluster within the melanoma patients was studied in the 4 cohorts analyzed and we confirmed an uneven distribution at this level (FIG. 26b). In particular, the beneficial supercluster 2, characterized by abundance of Ruminococceae was present in the Houston and New York cohorts in which members of this family and Faecalibacterium prausnitzzii in particular were observed to be associated with anti-PD-1 benefit, whereas it was almost absent in the Pittsburgh and Chicago cohorts. The beneficial supercluster 1, characterized by members of the Lachnospiraceae and Bifidobacteriaceae families, was abundant in the Pittsburgh and Chicago cohorts in which Lachnospiraceae and Bifidobacteriaceae spp., respectively, were found to be associated with clinical response.

### Example 10

In addition to many tumor-intrinsic mechanisms of response or resistance to anti-PD-1 (Zarour, H. M. 2016), intestinal microbiome composition appears to play a critical role in regulating responses to anti-PD-1 in cancer patients. Strikingly, different microbial species were implicated across various studies. Despite these apparent inconsistencies, responder-derived FMT have demonstrated efficacy in two independent proof-of-concept studies in anti-PD-1-refractory melanoma patients (Baruch, E. N., et al. 2021; Davar, D., et al. 2021). Many variables may explain the discordant results published so far, including population-specific characteristics, inter-individual variability, and the use of different molecular and bioinformatic approaches. To this end, the largest-to-date sampled cohort from the Hillman Cancer Center of the University of Pittsburgh was analyzed, along with a meta-analysis of publicly available data using the same assembly-based bioinformatic pipeline (JAMS) and restricted our analysis to PD-1-tretade melanoma patients.

The study herein evaluated whether baseline fecal microbiome composition predicted objective response at 3 months or progression free survival (PFS) at multiple timepoints during the first 30 months in melanoma patients treated anti-PD-1. Using time serial PERMANOVA, it was observed that maximal separation of Ps and NPs in the Pittsburgh cohort occurred at 9-10 months from start of therapy while at earlier or later time points a less significant separation was observed. An independent cohort from MD Anderson Cancer Center in Houston with available time-to-event data provided a similar optimal separation of the fecal microbiota of Ps and NPs at 13 months. Overall, these data indicate that gut microbial signatures predict PFS at 9-13 months across two independent cohorts of melanoma patients treated with anti-PD-1. This is consistent with the clinical findings that the majority of melanoma patients treated with first line anti-PD-1 alone may show disease progression up to approximately a year from start of therapy then approaching a plateau with a decreasing risk of subsequent relapse after 1 to 2 years (Robert, C., et al. 2018; Hamid, O., et al. 2019). Thus, these findings regarding the association of the clinical response at the time of most significant association with baseline microbiome composition, together with time-to-event analysis for the duration of patients' observation as well as the maintenance of a favorable microbiota composition in long-term NPs, show that the intestinal microbiota can influence both initial and sustained response to anti-PD-1. Whether the fecal microbiota composition changes at the time of the eventual progression observed in some long-term NPs remains to be determined. The precise effect size of the contribution of the microbiota to anti-PD-1 response and its relation to other biomarkers of primary and secondary resistance to anti-PD-1 in melanoma patients will need to be further validated in larger prospective studies.

This study has identified beneficial and detrimental signatures in baseline fecal microbiota composition using two separate biostatistical approaches on metagenomic sequencing data from the Pittsburgh cohort. Using Mann-Whitney U test in Ps and NPs at 10 months, the optimal time established by serial PERMANOVA, it is shown that increased abundance of Ruminococcus (Mediterraneibacter) torques, Ruminococcus (Mediterraneibacter) gnavus, Blautia wexlerae, Blautia hansenii, Eubacterium rectale, and Actinomyces bouchesdurhonensis was associated with favorable outcomes. In contrast, increased abundance of Alistipes spp., Prevotella spp., Bacteroides spp. and Oscillibacter spp. was associated with progression on anti-PD-1. To utilize the statistical power of the entire time-to-event data in the Pittsburgh cohort, this study has also identified beneficial and detrimental taxa by an alternative method using Cox regression analysis in Evaluate Cutpoints36 software that determines optimal fecal abundance cut points for all organisms and calculate their HRs in relation to PFS. The latter method, in addition to the taxa also identified by Mann-Whitney U test, identified many additional taxa associated with either improved or decreased PFS. Altogether, these data show that a beneficial gut microbiota signature, including multiple members of Actinobacteria phylum and Lachnospiraceae family of Firmicutes phylum, is associated with a favorable clinical outcome in a large cohort of melanoma patients treated with anti-PD-1. They also show that a detrimental signature—including multiple members of Bacteroidetes and Proteobacteria phyla as well as, within the Firmicutes phylum, members of the Streptococcaceae and Veillonellaceae families—is associated with progression upon anti-PD-1.

Microbial genome composition performed similarly to bacterial taxonomy in segregating Ps and NPs. The detailed interrogation of individual genes differentially abundant in Ps and NPs showed that the majority of these were responsible for bacterial housekeeping functions, such as DNA replication and basic metabolic processes. However, specific pathways, such as iron uptake and LPS metabolism with potential connections to host, were identified through careful gene-by-gene manual analysis of significantly differentially enriched genes in Ps and NPs. These findings can be explained by the fact that when bacterial taxa are associated with an outcome, all genes in their genome are likely to be as well. Thus, it is important to identify candidate “bacterial driver” genes, the products of which may directly affect the ability of the host to respond to the therapy. This can be accomplished by comparing genetically closely related bacteria taxa, such as strains, that have differential association with favorable or unfavorable anti-PD-1 clinical response to filter out most passenger genes and to focus on putative driver genes. To overcome the limitations inherent in reference databases that preclude efficient strain level analyses, a novel binning approach was developed for unbiased segregation of sequencing data into taxonomic units. Using this method, enrichment of comparable genes were observed in bacterial strains that belong to different parental species but were similarly associated with modulation of clinical outcome upon anti-PD-1. These included: genes associated with reduction of polysaccharide immunogenicity (polysaccharide deacytylase (Balomenou, S., et al. 2013)) in NPs; genes associated with mucus degradation (alpha-1-fucosidase (Fan, S., et al. 2016) and alpha-galactosidase (Wright, D. P., 2000)), and LPS synthesis (glycosyltransferases (Luke, N. R., et al. 2021)) in Ps. Collectively, these findings show that unrelated intestinal bacterial species exert similar functions, contributing to immune responses to anti-PD-1 in melanoma.

In agreement with published findings, baseline NLR was adversely associated with PFS and OS in the Pittsburgh melanoma cohort. NLR was independently associated with baseline gut microbiota composition. The taxa connected with high NLR were predominantly gram-negative and associated with disease progression. To understand how the intestinal microbiome correlated with host systemic inflammation, intestinal gene expression was evaluated via RNA sequencing of host cells in stool samples to interrogate host intestinal cell gene expression (Knight, J. M., et al. 2014). Among the most differentially expressed host genes in Ps were genes associated with an inflammatory and oxidative burst signature (IL1B, NFKB1Z, NFKB1A, CXCL8, L1TAF, TNFAP3, and SOD2). GSEA analysis indicated that these genes in addition to epithelial cells were expressed in host myeloid cells shed in the gut lumen such as neutrophils, dendritic cells, macrophages and monocytes. Conversely, NPs exhibited transcriptomic gene signatures associated with resolution of mucosal inflammation and encoding membrane associated mucins (MUC13, MUC20) and apolipoproteins (APOA1, APOA4, and APOB). Genes encoding several cytokines either directly or indirectly implicated in immune suppression were also upregulated in Ps, including CXCL8/IL-874-76, IL-1P77 and TNF78. Transkingdom network analyses indicated that the microbiota governed changes in host phenotype (PFS and NLR) and linked the presence of detrimental taxa to an LPS-dependent inflammatory signature.

This study reproduced previously reported associations between irAEs and improved PFS79-81, and found that baseline gut microbiome composition distinctly segregated PD-1-treated patients who developed irAEs from those who did not. Two distinct microbial signatures were associated with irAEs. The first is represented by members of Lachnospiraceae family that are largely associated with a favorable response to anti-PD-1 and are predominantly linked to thyroid, cutaneous, gastrointestinal and neurologic irAEs. The second, dominated by Streptococcus spp., is linked with pulmonary, joint and hepatic manifestations and associated with shorter PFS than the former. Streptococcus spp. are generally found in the upper gastrointestinal tract in humans. These observations regarding association of PPI use and Streptococcus spp. abundance in patients with irAEs suggest that the former increases survival rate of oral bacteria during gastric passage (i.e. oralization) resulting in a shift in gut microbiota composition. Streptococcus spp. has been related to multiple inflammatory joint disorders including osteomyelitis (Murillo, O., et al. 2014), osteoarthritis (Boer, C. G., et al. 2019), rheumatic fever (Cunningham, M. W. 2014; Tandon, R., et al. 2013), and post-streptococcal reactive arthritis (Barash, J. 2013). Pathogenic group A Streptococcal antigens induce cross-reactive antibodies that mediate joint inflammation underlying autoimmunity (Tandon, R., et al. 2013). However, the intestinal Streptococcus spp. Associated with colitis in this study are typically commensals that may acquire pathobiont characteristics in patients. irAE occurrence in melanoma patients with high fecal abundance of Streptococcus spp. indicates loss of disease tolerance to pathobionts (McCarville, J. L. & Ayres, J. S. 2018). Intestinal oralization with commensal microbes induced by PPI use conjoined to ICB-amplified anti-commensal immune responses can result in autoimmune pathology in part due to molecular mimicry (Rose, N. R. 2017; Yumoto, H., et al. 2019). Altogether, these findings can explain the discordant results linking irAEs and clinical response to anti-PD-1. While certain bacteria enhance the immunostimulatory effect of anti-PD-1 both against cancer and self antigens, others types induce organ pathology while suppressing cancer immunity. Depending on the predominant intestinal microbial composition in PD-1-treated melanoma patients, an association between irAEs and clinical response may or may not be observed.

Prior attempts at consolidating discrepant findings in studies of microbiome modulation of ICB response focused on combining multiple datasets across existing ICB-treated cohorts to increase analytic power. These meta-analyses included patients from different countries, multiple histologies and/or treatment with anti-PD-1, anti-CTLA-4 or their combination (Shaikh, F. Y., et al. 2021; Gharaibeh, R. Z. & Jobin, C. 2019; Limeta, A., 2020). Thus, such studies were hindered by significant between-study heterogeneity. In order to overcome these difficulties and to increase homogeneity, this study evaluated carefully selected cohorts of melanoma patients from the continental United States, treated with anti-PD-1, and used the same taxonomic classification tools along with sequence assembly to increase the robustness of analysis.

It was observed that higher alpha-diversity correlated with improved outcomes in only one of the five cohorts and failed to identify any discernable differences between several diversity metrics in the other cohorts. These findings indicate that general features of alpha-diversity may not play a major role in mediating response to anti-PD-1 in melanoma patients.

To increase the reproducibility in the identification of bacterial taxa associated with clinical response, four separate statistical methods of analysis of the combined data were used. Two approaches (Mann-Whitney and LDA) involved analyses of the five combined datasets after removal of study-related batch effects. Two meta-analytic techniques were also used: Fisher's method for combining p-values and a random-effects model. All these approaches revealed similar signatures of beneficial and detrimental microbiota. In particular, members of Lachnospiraceae and Ruminococcaceae families of Firmicutes phylum and several members of Actinobacteria phylum, including Bifidobacterium spp. were associated with favorable outcome to anti-PD-1 therapy. Conversely, members of Bacteroidetes phylum, especially Prevotellaceae, Bacteroidaceae and Rikenellaceae families were associated with unfavorable outcomes to anti-PD-1 in melanoma patients. These findings recapitulated observations made in the context of a clinical trial testing FMT to treat PD-1 refractory melanoma (Davar, D., et al. 2021). In general, beneficial bacterial taxa tended to be gram-positive while detrimental taxa tended to be gram-negative, supporting the idea that higher levels of LPS production can be associated with adverse outcomes. Machine learning analyses showed that models trained to predict response from microbial taxa abundance often worked well within a dataset but were poorly generalizable across datasets.

The inter-individual variation of the human gut microbiome is large (He, Y., et al. 2018; Gevers, D., et al. 2012; Human Microbiome Project, 2012). Multiple factors significantly affect its composition, including geography, diet, infections and drug usage (Asnicar, F., et al. 2021). Large multinational studies have shown that intestinal microbiota variation is stratified into discrete microbial clusters or enterotypes (Arumugam, M., et al. 2011). However, studies utilizing different clustering methods have questioned the concept of enterotypes and indicated that their distribution is continuous and their composition can vary widely within an individual (Gorvitovskaia, A., 2016; Knights, D., et al. 2014). Complexity of highly dimensional gut microbiota can be better comprehended by organization into discrete classes of ecologically balanced communities that consider their functional and ecological context—“enteric microbiotypes” (Arumugam, M., et al. 2011; Costea, P. I., et al. 2018). Enteric microbiotypes are distinctive of different geographical areas as the human gut microbiome is affected by ethnicity and geographical origin (Brooks, A. W., 2018; Dwiyanto, J., et al. 2021; Zhang, R., 2021). To overcome the limitation of a relatively small sample set, we constructed an “AGP enteric microbiotype map” using approximately 7,000 16S sequenced stool samples from AGP cohort limited to continental United States (McDonald, D., et al. 2018), and identified many different enteric microbiotypes. Delineating enteric microbiotypes by geography clarified that nearly all microbiotypes were unequally distributed in different States. While some of the enteric microbiotypes, were reported earlier, such as those dominated by Prevotella copri, others were not, such as those dominated by Akkermansia muciniphila, Bacteroides massiliensis, Ruminococcus bromii, Bifidobacterium spp., Peptoniphilus duerdenii, and Oceanobacillus indicireducens. Placement of 16S sequenced samples from four anti-PD-1 treated melanoma cohorts, for which 16S data were available, onto the AGP microbiotype map revealed that the representation of different microbiotypes in the 4 cohorts was unequal and this may explain, at least in part, inconsistencies in the identification of bacterial taxa associated with PD-1 response in the various studies. By calculating odds ratios (ORs) of response to non-response for the samples within each microbiotype, four distinct superclusters were identified grouping enteric microbiotypes with similar association between microbiome composition and clinical outcome. Two main beneficial superclusters were identified—one dominated by Lachospiraceae and Bifidobacteriaceae families of bacteria and Akkermansia muciniphila, and another by Ruminococcaceae including Faecalibacterium prausnitzii. Conversely, the two detrimental superclusters were characterized by bacteria in the Bacteriodales order with members of the Bacteroidaceae/Rickenellaceae and Prevotellaceae families dominating the first and second detrimental superclusters, respectively. The distribution of the different microbiotypes and superclusters in the 4 melanoma patient cohorts studied is compatible with a possible bias of observing a more significant association with a positive clinical response of members of the Ruminococcaceae family in Houston and New York, of the Bifidobacteriaceae family in Chicago and of the Lachnospiraceae family in Pittsburgh. Overall, the contextualization of gut microbiota samples through the lens of enteric microbiotypes permitted the identification of distinct beneficial and detrimental microbiota signatures in relation to anti-PD-1 and provides a roadmap to account for microbiota variability in the cohorts analyzed in future studies.

This study has identified in five separate cohorts of PD-1-treated melanoma shared bacterial families associated with either beneficial or detrimental effects on therapy response. The identification of different representatives from these families across different studies shows that they can dominate the enteric microbiotypes present in patients from different geographical origins, and that effective responses to anti-PD-1 can require the presence only few of the beneficial taxa. Beneficial bacteria, whether from the same or distinct families, can modulate the anti-cancer response by different mechanisms. However, the observation that beneficial strains across different species may share driver genes also indicates common mechanisms of action. The association of a bacterial taxa species with therapeutic response does not necessarily identify causality (Tjalsma, H. 2012). Bacterial species exist in an ecological equilibrium being co-expressed in the same patients, and consequently neutral “passenger” bacterial taxa may be identified together with bacterial taxa that have a casual association with the clinical response. Alternatively, neutral “keystone” bacterial taxa can impact therapeutic response by regulating the colonization of other detrimental or beneficial taxa. In support of this hypothesis, PD-1 refractory melanoma patients who responded to FMT and anti-PD-1 exhibited not only changes of the gut microbiota due to the successful colonization of donor species, but also expansion or contraction of taxa of both donor and recipient origin, supporting that FMT induced changes in intestinal ecology (Davar, D., et al. 2021). In this study, a systemic inflammatory signature with myeloid-derived chemokines, such as IL-8, which is associated with PD-1 failure, was also suppressed or enhanced by beneficial and detrimental species, respectively (Davar, D., et al. 2021). This focused meta-analysis, which uncovered shared patterns of bacterial dominance in PD-1 Rs and NRs, can explain some of the apparently discordant results reported by various published studies. For example, Bacteroidetes spp. were associated with poor response in PD-1 treated melanoma patients, but with favorable responses in CTLA-4 treated melanoma patients and in tumor-bearing mice17. This observation indicates that specific tumors and ICBs are differentially affected by disparate bacterial taxa. In addition, the meta-analysis of PD-1 treated melanoma patients showed significant species-level variation, and the ability to predict response in each cohort trained on the microbiome data from other cohorts by machine learning was modest. The transkingdom analysis indicated that the detrimental taxa exerted more control over the therapeutic response and associated inflammatory/immunological profiles compared to beneficial taxa. This observation is consistent with a recent report that a machine learning approach that train taxa associated with a detrimental signature rather than those associated with a beneficial signature or with both is slightly more effective in predicting patients' response to therapy (Shaikh, F. Y., et al. 2021).

In summary, the findings herein provide a comprehensive evaluation of baseline gut microbial association with clinical response and occurrence of irAEs in PD-1 treated melanoma patients. Given the differential ability of bacterial strains to modulate response to ICB, via mechanisms that are either individual or shared among strains from different parental species, future studies should include high-resolution strain-level or subspecies-level analyses to capture this dimension. Further, given the reported variability of metagenomic composition by geography and lifestyle factors including diet, and drug usage, it is also be essential to account for these factors in determining enteric microbiotypes and their temporal modulation. The recent clinical demonstration that modifying the intestinal microbiome by FMT can improve patients' response to anti-PD-1 provides compelling evidence that the gut microbiome can be manipulated to improve current immunotherapy of cancer. The identification of bacterial taxa modulating the anti-cancer response and their mechanisms of action will provide the rationale for the development of novel microbial-based therapies of cancer (Baruch, E. N., et al. 2021; Davar, D., et al. 2021).

TABLE 1
Patient Characteristics (Pittsburgh cohorts)

| | | | Early Sample | | Late Sample | |
|---|---|---|---|---|---|---|
| Characteristics | Total (n = 94) | | Cohort (n = 63) | | Cohort (n = 31) | |
| Age (Years): Median, range | 70 | (32-90) | 67 | (32-90) | 74 | (41-89) |
| Sex: | | | | | | |
| Male | 66 | (70%) | 42 | (67%) | 24 | (77%) |
| Race: | | | | | | |
| Caucasian | 94 | (100%) | 63 | (100%) | 31 | (100%) |
| Body Mass Index: | | | | | | |
| Males: Median, range | 29 | (19-43) | 28 | (19-43) | 29 | (20-42) |
| Females: Median, range | 30 | (18-42) | 32 | (18-42) | 29 | (25-42) |
| Melanoma Stage IV Grouping: | | | | | | |
| M1a | 29 | (31%) | 18 | (29%) | 11 | (36%) |
| M1b | 20 | (21%) | 14 | (22%) | 6 | (19%) |
| M1c | 34 | (36%) | 24 | (38%) | 10 | (32%) |
| M1d | 1 | (12%) | 7 | (11%) | 4 | (13%) |
| Lactate Dehydrogenase: | | | | | | |
| Median, range (U/ml) | 202 | (136-1108) | 202 | (141-603) | 202 | (136-1108) |
| Ratio to high normal (171 u/ml) | 1.2 | (0.8-6.5) | 1.8 | (0.8-3.5) | 1.2 | (0.8-6.5) |
| Neutrophil Lymphocyte Ratio: | | | | | | |
| Median, range | 2.7 | (1.1-17.3) | 2.7 | (1.3-17.3) | 2.7 | (1.1-11.3) |
| Prior therapy: | | | | | | |
| None | 78 | (83%) | 60 | (95%) | 18 | (58%) |
| Anti-CTLA-4 | 13 | (14%) | 3 | (5%) | 10 | (32%) |
| High Dose IL-2 | 3 | (3%) | 0 | | 3 | (10%) |
| BRAF/MEK Inhibitors | 4 | (4%) | 1 | (2%) | 3 | (10%) |
| Melanoma Vaccine | 1 | (1%) | 0 | | 1 | (3%) |
| Immunotherapy type: | | | | | | |
| Nivolumab | 23 | (25%) | 18 | (29%) | 5 | (16%) |
| Pembrolizumab | 48 | (51%) | 29 | (46%) | 19 | (61%) |
| Pembrolizumab/PEG-Interferon | 21 | (22%) | 14 | (22%) | 7 | (23%) |
| Anti-PD-1 (investigational) | 2 | (2%) | 2 | (3%) | 0 | |
| Treatment-emergent Adverse Events | | | | | | |
| None | 36 | (38%) | 23 | (37%) | 13 | (42%) |
| Any | 58 | (62%) | 40 | (63%) | 18 | (58%) |
| Highest Grade Event | | | | | | |
| Grade 1 | 4 | (4%) | 2 | (3%) | 2 | (7%) |
| Grade 2 | 27 | (29%) | 21 | (33%) | 6 | (19%) |
| Grade 3-4 | 27 | (29%) | 17 | (27%) | 10 | (32%) |

TABLE 2
Cox Regression Models on Overall Survival and Progression-
Free Survival (Pittsburgh Early Sample Cohort)

| | Overall Survival | | | Progression Free Survival | |
|---|---|---|---|---|---|
| | Univariate | Multivariate | | Univariate | Multivariate |
| | HR, 95% CI, | HR, 95% CI, | | HR, 95% CI, | HR, 95% CI, |
| Factors | p-value | p-value | Factors | p-value | p-value |
| Sex (M vs F) | 1.31 | | Sex (M vs F) | 1.36 | |
| | (0.578-2.99), | | | (0.649-2.85), | |
| | P = 0.513 | | | P = 0.413 | |
| Age in years | 1.71 | | Age in years | 0.55 | |
| (<76.51 vs ≥76.51) | (0.799-3.65), | | (<56.02 | (0.238-1.27), | |
| | P = 0.162 | | vs ≥56.02) | P = 0.154 | |
| BMI | 0.388 | 0.424 | BMI | 0.552 | |
| (<26.4 vs ≥26.4) | (0.184-0.82), | (0.198-0.909), | (<26.4 | (0.278-1.09), | |
| | P = 0.010 | P = 0.028 | vs ≥26.4) | P = 0.084 | |
| Lines of treatment | 1.79 | | Lines of | 1.25 | |
| (0 vs ≥1) | (0.423-7.56), | | treatment | (0.299-5.22), | |
| | P = 0.423 | | (0 vs ≥1) | P = 0.759 | |
| PPI usage | 2.07 | | PPI usage | 2.12 | 2.197 |
| | (0.907-4.71), | | | (0.988-4.56), | (1.01-4.80), |
| | P = 0.078 | | | P = 0.048 | P = 0.0483 |
| LDH ratio | 2.24 | | LDH ratio | 1.43 | |
| (<1.69 vs ≥1.69) | (0.946-5.28), | | (<1.368 | (0.706-2.89), | |
| | P = 0.060 | | vs ≥1.368) | P = 0.318 | |
| Neutrophil to | 2.4 | 2.227 | Neutrophil to | 2.53 | 2.443 |
| lymphocyte ratio | (1.11-5.19), | (1.025-4.841), | lymphocyte ratio | (1.23-5.17), | (1.19-5.01), |
| (≥3.75 vs <3.75) | P = 0.022 | P = 0.043 | (≥3.75 | P = 0.009 | P = 0.015 |
| | | | vs <3.75) | | |

TABLE 3
Characteristics of melanoma patient cohorts from previously published studies

| | | Patient Characteristics | | Database | Duration of | Definition of |
|---|---|---|---|---|---|---|
| Study | Region | (response breakdwon) | Analytic Pipeline | Corrections | Follow-up | response |
| Peters et | New | 14 metastatic melanoma | 16S | Taxa | Start of | PD or death |
| al., 2019 | York | patients on anti- PD1 | sequencing: | unidentified at | therapy to | from any |
| | | therapy (7 non- | QIIME 2, | the genus or | progression, | cause vs non |
| | | progressors, 7 | Greengenes | species level | death, or loss | progression |
| | | progressors) | database | removed | to follow up | during |
| | | Fecal samples analyzed | Shotgun | | (10-25 | follow-up |
| | | by 16S and shotgun | sequencing: | | months) | period |
| | | metagenomic + | MetaPhlAn2, | | | |
| | | metatranscriptomic | HUMAnN2 | | | |
| | | sequencing | | | | |
| Frankel et | Dallas | 14 non-resectable or | MetaPhlAn, | | Not reported | Response by |
| al., 2017 | | metastatic melanoma | HUMAN and | | | RECIST |
| | | patients on anti-PD1 | FMAP | | | v1.1 |
| | | therapy (7 | | | | (CR/PR/SD |
| | | nonprogressors, 7 | | | | vs. PD, |
| | | progressors) | | | | evaluated at |
| | | Fecal samples analyzed | | | | 3-6 month |
| | | by shotgun metagenomic | | | | intervals) |
| | | sequencing | | | | |
| Matson et | Chicago | 38 metastatic melanoma | 16S | Composite | Not reported | Response by |
| al., 2018 | | patients on anti- PD1 | sequencing: | analysis- | | RECIST |
| | | therapy +4 patients on | QIIME, NCBI | Species | | v1.1 (CR/PR |
| | | anti-CTLA-4 therapy (16 | database | identified in | | any time |
| | | responders, 26 non- | Shotgun | 16S, qPCR, | | during the |
| | | responders) | sequencing: | and shotgun | | study vs. no |
| | | Fecal samples from 42 | MetaPhlAn2 | were retained | | response) |
| | | patients analyzed by 16S | | | | |
| | | sequencing | | | | |
| | | Fecal samples from 39 | | | | |
| | | patients analyzed by | | | | |
| | | shotgun metagenomic | | | | |
| | | sequencing (15 | | | | |
| | | responders, 24 non- | | | | |
| | | responders) | | | | |
| Gopalakrishnan | Houston | 43 metastatic melanoma | 16S | 16S restricted | Not reported | Response by |
| et al., 2018 | | patients on anti- PD1 | sequencing: | to family level | | RECIST |
| | | therapy (30 responders, | QIIME, | | | v1.1 (CR/PR |
| | | 13 non-responders) | Greengenes, | | | or SD > 6 |
| | | Fecal samples from 43 | RDP, and Silva | | | months vs. |
| | | patients analyzed by 16S | databases | | | PD or SD < 6 |
| | | sequencing | Shotgun | | | months) |
| | | Fecal samples from 25 | sequencing: | | | |
| | | patients analyzed by | MetaOMineR | | | |
| | | shotgun metagenomic | | | | |
| | | sequencing (14 | | | | |
| | | responders, 11 non- | | | | |
| | | responders) | | | | |

CR = complete response, PR = partial response, PD = progressive disease, SD = stable disease, FMAP = Functional Mapping and Analysis Pipeline, RECIST = Response Evaluation Criteria In Solid Tumors-version 1.1., QIIME = Quantitative Insights Into Microbial Ecology, MetaPhlAn: Metagenomic Phylogenetic Analysis, HUMAnN = HMP Unified Metabolic Analysis Network, NCBI = National Center for Biotechnology Information, RDP = Ribosomal Database Project, MetaOMineR = Mining Metaomics Data.

TABLE 4
Taxa Associated with Improved PFS (Pittsburgh Early Sample Cohort)

| | | Biomarker < | | Biomarker >= | | | | P- | |
|---|---|---|---|---|---|---|---|---|---|
| LKT | Cutpoint | Cutpoint | | Cutpoint | | HR | CI | value | FDR |
| LKT_s_Blautia_coccoides | 11 | 16 | (25%) | 47 | (75%) | 0.292 | (0.146-0.585) | 0 | 0.016 |
| LKT_f_Lachnospiraceae | 7515 | 11 | (17%) | 52 | (83%) | 0.268 | (0.124-0.575) | 0 | 0.016 |
| LKT_s_Ruminococcus_torques | 3664 | 32 | (51%) | 31 | (49%) | 0.271 | (0.128-0.573) | 0 | 0.016 |
| LKT_s_Blautia_producta | 119 | 23 | (37%) | 40 | (63%) | 0.339 | (0.171-0.671) | 0.001 | 0.025 |
| LKT_s_Absiella_dolichum | 4 | 32 | (51%) | 31 | (49%) | 0.329 | (0.16-0.679) | 0.002 | 0.025 |
| LKT_s_Enterocloster_bolteae | 289 | 17 | (27%) | 46 | (73%) | 0.345 | (0.173-0.692) | 0.002 | 0.026 |
| LKT_s_Bifidobacterium_bifidum | 10 | 22 | (35%) | 41 | (65%) | 0.361 | (0.183-0.709) | 0.002 | 0.027 |
| LKT_f_Erysipelotrichaceae | 247 | 18 | (29%) | 45 | (71%) | 0.351 | (0.175-0.702) | 0.002 | 0.027 |
| LKT_s_Anaerostipes_hadrus | 3690 | 29 | (46%) | 34 | (54%) | 0.358 | (0.178-0.719) | 0.003 | 0.029 |
| LKT_s_Lachnospiraceae_bacterium | 8746 | 23 | (37%) | 40 | (63%) | 0.372 | (0.189-0.733) | 0.003 | 0.03 |
| LKT_s_Clostridium_scindens | 77 | 20 | (32%) | 43 | (68%) | 0.373 | (0.189-0.736) | 0.003 | 0.03 |
| LKT_s_Enterorhabdus_caecimuris | 39 | 52 | (83%) | 11 | (17%) | 0.0975 | (0.0133-0.714) | 0.005 | 0.037 |
| LKT_s_Eisenbergiella_massiliensis | 220 | 34 | (54%) | 29 | (46%) | 0.357 | (0.17-0.75) | 0.005 | 0.037 |
| LKT_s_Collinsella_intestinalis | 22 | 48 | (76%) | 15 | (24%) | 0.212 | (0.0646-0.695) | 0.005 | 0.038 |
| LKT_s_Actinomyces_oris | 15 | 24 | (38%) | 39 | (62%) | 0.392 | (0.199-0.772) | 0.005 | 0.039 |
| LKT_g_Coprococcus | 49 | 26 | (41%) | 37 | (59%) | 0.393 | (0.199-0.776) | 0.005 | 0.04 |
| LKT_s_Enterocloster_clostridioformis | 726 | 17 | (27%) | 46 | (73%) | 0.391 | (0.194-0.788) | 0.007 | 0.046 |
| LKT_s_Lactococcus_lactis | 4 | 12 | (19%) | 51 | (81%) | 0.363 | (0.168-0.783) | 0.007 | 0.049 |
| LKT_s_Blautia_hydrogenotrophica | 45 | 15 | (24%) | 48 | (76%) | 0.398 | (0.196-0.809) | 0.008 | 0.051 |
| LKT_s_Bacteroides_eggerthii | 323 | 35 | (56%) | 28 | (44%) | 0.383 | (0.183-0.804) | 0.008 | 0.051 |
| LKT_s_Blautia_hansenii | 48 | 12 | (19%) | 51 | (81%) | 0.377 | (0.178-0.799) | 0.008 | 0.051 |
| LKT_s_Candidatus_Stoquefichus | 3 | 17 | (27%) | 46 | (73%) | 0.41 | (0.204-0.821) | 0.009 | 0.054 |
| LKT_s_Dorea_longicatena | 17720 | 53 | (84%) | 10 | (16%) | 0.116 | (0.0158-0.849) | 0.011 | 0.057 |
| LKT_s_Coprococcus_comes | 251 | 12 | (19%) | 51 | (81%) | 0.397 | (0.184-0.86) | 0.015 | 0.066 |
| LKT_g_Blautia | 965 | 17 | (27%) | 46 | (73%) | 0.433 | (0.215-0.873) | 0.016 | 0.068 |
| LKT_s_Blautia_obeum | 1024 | 13 | (21%) | 50 | (79%) | 0.417 | (0.198-0.878) | 0.018 | 0.073 |
| LKT_s_Firmicutes_bacterium | 4698 | 47 | (75%) | 16 | (25%) | 0.31 | (0.109-0.881) | 0.02 | 0.078 |
| LKT_s_Clostridiaceae_bacterium | 2698 | 25 | (40%) | 38 | (60%) | 0.458 | (0.233-0.902) | 0.021 | 0.079 |
| LKT_s_Bacteroides_thetaiotaomicron | 22330 | 52 | (83%) | 11 | (17%) | 0.216 | (0.0517-0.902) | 0.021 | 0.079 |
| LKT_s_Blautia_Unclassified | 3331 | 15 | (24%) | 48 | (76%) | 0.438 | (0.212-0.902) | 0.021 | 0.08 |
| LKT_s_Anaerostipes_caccae | 7 | 25 | (40%) | 38 | (60%) | 0.462 | (0.235-0.908) | 0.022 | 0.08 |
| LKT_s_Adlercreutzia_equolifaciens | 621 | 44 | (70%) | 19 | (30%) | 0.372 | (0.154-0.901) | 0.023 | 0.082 |
| LKT_s_Lactobacillus_crispatus | 4 | 33 | (52%) | 30 | (48%) | 0.451 | (0.223-0.914) | 0.023 | 0.083 |
| LKT_s_Clostridium_methylpentosum | 149 | 40 | (63%) | 23 | (37%) | 0.415 | (0.188-0.92) | 0.026 | 0.085 |
| LKT_s_Fusicatenibacter_saccharivorans | 3315 | 40 | (63%) | 23 | (37%) | 0.415 | (0.188-0.919) | 0.025 | 0.085 |
| LKT_s_Adlercreutzia_Unclassified | 358 | 40 | (63%) | 23 | (37%) | 0.417 | (0.188-0.923) | 0.026 | 0.085 |
| LKT_s_Coprobacillus_Unclassified | 12 | 10 | (16%) | 53 | (84%) | 0.402 | (0.174-0.928) | 0.027 | 0.087 |
| LKT_s_Enterocloster_aldensis | 236 | 51 | (81%) | 12 | (19%) | 0.285 | (0.087-0.935) | 0.027 | 0.087 |
| LKT_s_Blautia_wexlerae | 12600 | 41 | (65%) | 22 | (35%) | 0.421 | (0.19-0.933) | 0.028 | 0.088 |
| LKT_s_Mogibacterium_diversum | 9 | 35 | (56%) | 28 | (44%) | 0.456 | (0.221-0.938) | 0.029 | 0.089 |
| LKT_s_Streptococcus_thermophilus | 16 | 17 | (27%) | 46 | (73%) | 0.468 | (0.231-0.951) | 0.032 | 0.096 |
| LKT_s_Parabacteroides_johnsonii | 338 | 49 | (78%) | 14 | (22%) | 0.342 | (0.12-0.974) | 0.035 | 0.101 |
| LKT_g_Dorea | 26 | 22 | (35%) | 41 | (65%) | 0.497 | (0.251-0.981) | 0.04 | 0.11 |
| LKT_s_Dorea_Unclassified | 2357 | 53 | (84%) | 10 | (16%) | 0.251 | (0.06-1.05) | 0.041 | 0.11 |
| LKT_s_Eubacterium_rectale | 1520 | 13 | (21%) | 50 | (79%) | 0.462 | (0.214-0.996) | 0.044 | 0.113 |
| LKT_s_Gordonibacter_urolithinfaciens | 91 | 27 | (43%) | 36 | (57%) | 0.507 | (0.258-0.995) | 0.044 | 0.114 |
| LKT_s_Schaalia_odontolytica | 44 | 44 | (70%) | 19 | (30%) | 0.436 | (0.189-1) | 0.045 | 0.115 |
| LKT_s_Ruminococcusgnavus | 5102 | 45 | (71%) | 18 | (29%) | 0.417 | (0.172-1.01) | 0.046 | 0.115 |
| LKT_f_Akkermansiaceae | 17 | 36 | (57%) | 27 | (43%) | 0.489 | (0.238-1) | 0.047 | 0.116 |
| LKT_f_Eggerthellaceae | 97 | 15 | (24%) | 48 | (76%) | 0.492 | (0.239-1.01) | 0.048 | 0.117 |
| LKT_s_Parabacteroides_distasonis | 5275 | 29 | (46%) | 34 | (54%) | 0.515 | (0.261-1.02) | 0.051 | 0.118 |
| LKT_s_Asaccharobacter_celatus | 358 | 43 | (68%) | 20 | (32%) | 0.449 | (0.195-1.03) | 0.054 | 0.12 |
| LKT_s_Sellimonas_intestinalis | 279 | 35 | (56%) | 28 | (44%) | 0.509 | (0.252-1.03) | 0.056 | 0.123 |
| LKT_s_Mogibacterium_pumilum | 3 | 44 | (70%) | 19 | (30%) | 0.457 | (0.199-1.05) | 0.058 | 0.127 |
| LKT_s_Eggerthella_lenta | 286 | 22 | (35%) | 41 | (65%) | 0.525 | (0.266-1.04) | 0.059 | 0.127 |
| LKT_s_Erysipelatoclostridium_ramosum | 21 | 33 | (52%) | 30 | (48%) | 0.52 | (0.26-1.04) | 0.06 | 0.129 |
| LKT_s_Eubacteriaceae_bacterium | 280 | 16 | (25%) | 47 | (75%) | 0.515 | (0.254-1.04) | 0.061 | 0.13 |
| LKT_s_Catenibacterium_mitsuokai | 4 | 32 | (51%) | 31 | (49%) | 0.528 | (0.266-1.05) | 0.063 | 0.134 |
| LKT_s_Collinsella_stercoris | 8 | 40 | (63%) | 23 | (37%) | 0.494 | (0.23-1.06) | 0.065 | 0.135 |
| LKT_s_Actinomyces_bouchesdurhonensis | 3 | 31 | (49%) | 32 | (51%) | 0.529 | (0.266-1.05) | 0.065 | 0.135 |
| LKT_g_Anaerostipes | 256 | 50 | (79%) | 13 | (21%) | 0.393 | (0.138-1.12) | 0.069 | 0.141 |
| LKT_s_Anaeromassilibacillus_Unclassified | 307 | 40 | (63%) | 23 | (37%) | 0.503 | (0.234-1.08) | 0.072 | 0.146 |
| LKT_s_Tyzzerella_nexilis | 39 | 19 | (30%) | 44 | (70%) | 0.536 | (0.268-1.07) | 0.073 | 0.147 |
| LKT_s_Blautia_schinkii | 25 | 10 | (16%) | 53 | (84%) | 0.474 | (0.205-1.09) | 0.074 | 0.147 |
| LKT_s_Enterocloster_lavalensis | 80 | 40 | (63%) | 23 | (37%) | 0.506 | (0.236-1.09) | 0.075 | 0.148 |
| LKT_s_Faecalibacterium_prausnitzii | 52400 | 42 | (67%) | 21 | (33%) | 0.498 | (0.225-1.1) | 0.079 | 0.149 |
| LKT_s_Eubacterium_limosum | 10 | 44 | (70%) | 19 | (30%) | 0.48 | (0.209-1.1) | 0.077 | 0.149 |
| LKT_s_Anaerotruncus_Unclassified | 490 | 52 | (83%) | 11 | (17%) | 0.361 | (0.11-1.18) | 0.079 | 0.149 |
| LKT_s_Duodenibacillus_massiliensis | 1 | 20 | (32%) | 43 | (68%) | 0.55 | (0.278-1.09) | 0.082 | 0.149 |
| LKT_s_Faecalimonas_umbilicata | 79 | 43 | (68%) | 20 | (32%) | 0.5 | (0.226-1.11) | 0.082 | 0.149 |

TABLE 5
Taxa Associated with Decreased PFS (Pittsburgh Early Sample Cohort)

| | | Biomarker < | | Biomarker >= | | | | P- | |
|---|---|---|---|---|---|---|---|---|---|
| LKT | Cutpoint | Cutpoint | | Cutpoint | | HR | CI | value | FDR |
| LKT_g_Prevotella | 304 | 50 | (79%) | 13 | (21%) | 4.46 | (2.18-9.14) | 0 | 0.003 |
| LKT_s_Prevotella_koreensis | 2 | 49 | (78%) | 14 | (22%) | 3.54 | (1.73-7.26) | 0 | 0.016 |
| LKT_k_Bacteria | 8095 | 48 | (76%) | 15 | (24%) | 3.72 | (1.84-7.51) | 0 | 0.016 |
| LKT_s_Prevotella_copri | 127 | 37 | (59%) | 26 | (41%) | 3.45 | (1.72-6.89) | 0 | 0.016 |
| LKT_s_Prevotella_oryzae | 1 | 46 | (73%) | 17 | (27%) | 3.25 | (1.63-6.47) | 0 | 0.019 |
| LKT_s_Intestinimonas_butyriciproducens | 280 | 28 | (44%) | 35 | (56%) | 3.63 | (1.68-7.84) | 0 | 0.02 |
| LKT_s_Bacteroidales_bacterium | 15 | 46 | (73%) | 17 | (27%) | 3.01 | (1.49-6.09) | 0.001 | 0.025 |
| LKT_s_Alistipes_senegalensis | 226 | 36 | (57%) | 27 | (43%) | 2.96 | (1.48-5.9) | 0.001 | 0.025 |
| LKT_s_Subdoligranulum_Unclassified | 2806 | 50 | (79%) | 13 | (21%) | 3.22 | (1.54-6.7) | 0.001 | 0.025 |
| LKT_s_Sporobacter_termitidis | 39 | 38 | (60%) | 25 | (40%) | 3.05 | (1.53-6.08) | 0.001 | 0.025 |
| LKT_s_Streptomyces_Unclassified | 59 | 31 | (49%) | 32 | (51%) | 3.39 | (1.61-7.13) | 0.001 | 0.025 |
| LKT_g_Pseudoflavonifractor | 124 | 37 | (59%) | 26 | (41%) | 2.97 | (1.49-5.91) | 0.001 | 0.025 |
| LKT_s_Clostridium_perfringens | 218 | 52 | (83%) | 11 | (17%) | 3.25 | (1.53-6.91) | 0.001 | 0.025 |
| LKT_s_Veillonella_parvula | 7 | 36 | (57%) | 27 | (43%) | 3.12 | (1.55-6.27) | 0.001 | 0.025 |
| LKT_s_Oscillibacter_ruminantium | 267 | 42 | (67%) | 21 | (33%) | 2.94 | (1.49-5.82) | 0.001 | 0.025 |
| LKT_s_Prevotella_Unclassified | 345 | 48 | (76%) | 15 | (24%) | 3.11 | (1.54-6.28) | 0.001 | 0.025 |
| LKT_g_Haemophilus | 3 | 28 | (44%) | 35 | (56%) | 3.24 | (1.51-6.98) | 0.001 | 0.025 |
| LKT_s_Lactobacillus_fermentum | 18 | 42 | (67%) | 21 | (33%) | 2.86 | (1.45-5.64) | 0.002 | 0.025 |
| LKT_p_Proteobacteria | 366 | 38 | (60%) | 25 | (40%) | 2.85 | (1.44-5.64) | 0.002 | 0.026 |
| LKT_s_Streptococcus_mutans | 191 | 49 | (78%) | 14 | (22%) | 2.95 | (1.44-6.02) | 0.002 | 0.027 |
| LKT_s_Ruminococcus_flavefaciens | 962 | 21 | (33%) | 42 | (67%) | 3.99 | (1.54-10.3) | 0.002 | 0.027 |
| LKT_s_Prevotella_buccae | 6 | 37 | (59%) | 26 | (41%) | 2.77 | (1.4-5.48) | 0.002 | 0.028 |
| LKT_s_Parabacteroides_merdae | 5565 | 36 | (57%) | 27 | (43%) | 2.76 | (1.39-5.48) | 0.003 | 0.029 |
| LKT_s_Eubacterium_pyruvativorans | 3 | 16 | (25%) | 47 | (75%) | 5.17 | (1.57-17) | 0.003 | 0.029 |
| LKT_s_Oscillibacter_valericigenes | 241 | 50 | (79%) | 13 | (21%) | 2.88 | (1.39-5.99) | 0.003 | 0.03 |
| LKT_g_Bacillus | 89 | 50 | (79%) | 13 | (21%) | 2.9 | (1.39-6.03) | 0.003 | 0.03 |
| LKT_s_Cuneatibacter_caecimuris | 53 | 41 | (65%) | 22 | (35%) | 2.69 | (1.36-5.32) | 0.003 | 0.03 |
| LKT_s_Acinetobacter_baumannii | 98 | 38 | (60%) | 25 | (40%) | 2.64 | (1.34-5.19) | 0.004 | 0.034 |
| LKT_c_Actinobacteria | 157 | 38 | (60%) | 25 | (40%) | 2.64 | (1.33-5.22) | 0.004 | 0.035 |
| LKT_s_Porphyromonas_uenonis | 1 | 33 | (52%) | 30 | (48%) | 2.7 | (1.34-5.42) | 0.004 | 0.035 |
| LKT_s_Alistipes_dispar | 44 | 35 | (56%) | 28 | (44%) | 2.63 | (1.32-5.25) | 0.004 | 0.037 |
| LKT_Unclassified | 51400 | 33 | (52%) | 30 | (48%) | 2.66 | (1.32-5.35) | 0.004 | 0.037 |
| LKT_s_Christensenella_minuta | 21 | 29 | (46%) | 34 | (54%) | 2.78 | (1.32-5.86) | 0.005 | 0.039 |
| LKT_s_Prevotella_lascolaii | 5 | 39 | (62%) | 24 | (38%) | 2.51 | (1.27-4.93) | 0.006 | 0.044 |
| LKT_g_Veillonella | 23 | 36 | (57%) | 27 | (43%) | 2.52 | (1.27-5) | 0.006 | 0.045 |
| LKT_s_Streptococcus_vestibularis | 193 | 51 | (81%) | 12 | (19%) | 2.68 | (1.28-5.62) | 0.007 | 0.047 |
| LKT_s_Fournierella_massiliensis | 32 | 12 | (19%) | 51 | (81%) | 5.65 | (1.35-23.6) | 0.007 | 0.05 |
| LKT_s_Eubacterium_siraeum | 1209 | 33 | (52%) | 30 | (48%) | 2.53 | (1.25-5.13) | 0.008 | 0.05 |
| LKT_s_Agathobaculum_desmolans | 187 | 50 | (79%) | 13 | (21%) | 2.58 | (1.25-5.34) | 0.008 | 0.051 |
| LKT_s_Akkermansia_muciniphila | 294 | 27 | (43%) | 36 | (57%) | 2.64 | (1.25-5.56) | 0.008 | 0.051 |
| LKT_s_Oxalobacter_formigenes | 7 | 49 | (78%) | 14 | (22%) | 2.55 | (1.23-5.28) | 0.009 | 0.054 |
| LKT_o_Bacteroidales | 8281 | 46 | (73%) | 17 | (27%) | 2.47 | (1.22-4.98) | 0.009 | 0.054 |
| LKT_s_Victivallales_bacterium | 3 | 33 | (52%) | 30 | (48%) | 2.43 | (1.21-4.88) | 0.01 | 0.055 |
| LKT_s_Alistipes_finegoldii | 13370 | 54 | (86%) | 9 | (14%) | 2.87 | (1.24-6.65) | 0.01 | 0.056 |
| LKT_s_Bacteroides_ilei | 16 | 39 | (62%) | 24 | (38%) | 2.35 | (1.19-4.63) | 0.011 | 0.057 |
| LKT_s_Intestinimonas_massiliensis | 798 | 48 | (76%) | 15 | (24%) | 2.44 | (1.2- 4.95) | 0.011 | 0.057 |
| LKT_s_Lachnospira_eligens | 1316 | 40 | (63%) | 23 | (37%) | 2.35 | (1.19-4.62) | 0.011 | 0.057 |
| LKT_s_Bacteriophage_Unclassified | 11 | 21 | (33%) | 42 | (67%) | 3 | (1.24-7.26) | 0.011 | 0.057 |
| LKT_s_Odoribacter_splanchnicus | 2436 | 44 | (70%) | 19 | (30%) | 2.37 | (1.19-4.72) | 0.012 | 0.059 |
| LKT_s_Bacillus_Unclassified | 26 | 31 | (49%) | 32 | (51%) | 2.41 | (1.19-4.91) | 0.012 | 0.061 |
| LKT_s_Angelakisella_massiliensis | 110 | 18 | (29%) | 45 | (71%) | 3.16 | (1.22-8.19) | 0.012 | 0.061 |
| LKT_s_Haemophilus_parainfluenzae | 2 | 30 | (48%) | 33 | (52%) | 2.44 | (1.18-5.02) | 0.013 | 0.062 |
| LKT_s_Ruminococcus_bromii | 88 | 25 | (40%) | 38 | (60%) | 2.56 | (1.19-5.5) | 0.013 | 0.062 |
| LKT_s_Flintibacter_Unclassified | 22 | 19 | (30%) | 44 | (70%) | 2.92 | (1.21-7.08) | 0.013 | 0.062 |
| LKT_f_Sutterellaceae | 5 | 34 | (54%) | 29 | (46%) | 2.33 | (1.17-4.64) | 0.013 | 0.062 |
| LKT_s_Alistipes_shahii | 7306 | 47 | (75%) | 16 | (25%) | 2.39 | (1.18-4.87) | 0.013 | 0.062 |
| LKT_s_Bacteroides_caccae | 306 | 13 | (21%) | 50 | (79%) | 3.99 | (1.22-13.1) | 0.014 | 0.063 |
| LKT_s_Odoribacter_laneus | 18 | 46 | (73%) | 17 | (27%) | 2.34 | (1.16-4.68) | 0.014 | 0.063 |
| LKT_s_Alistipes_communis | 1115 | 37 | (59%) | 26 | (41%) | 2.27 | (1.15-4.47) | 0.015 | 0.064 |
| LKT_g_Acidaminococcus | 10 | 31 | (49%) | 32 | (51%) | 2.36 | (1.16-4.78) | 0.014 | 0.064 |
| LKT_s_Rikenellaceae_bacterium | 198 | 49 | (78%) | 14 | (22%) | 2.41 | (1.16-4.98) | 0.015 | 0.064 |
| LKT_s_Bacteroides_plebeius | 1291 | 53 | (84%) | 10 | (16%) | 2.61 | (1.17-5.81) | 0.015 | 0.064 |
| LKT_g_Klebsiella | 23 | 33 | (52%) | 30 | (48%) | 2.31 | (1.15-4.63) | 0.016 | 0.066 |
| LKT_s_Bacteroides_faecis | 1816 | 49 | (78%) | 14 | (22%) | 2.36 | (1.14-4.88) | 0.017 | 0.072 |
| LKT_s_Coprobacter_fastidiosus | 2 | 11 | (17%) | 52 | (83%) | 4.78 | (1.14-20) | 0.018 | 0.073 |
| LKT_g_Collinsella | 33 | 21 | (33%) | 42 | (67%) | 2.62 | (1.14-6.03) | 0.019 | 0.075 |
| LKT_s_Paenibacillus_Unclassified | 52 | 40 | (63%) | 23 | (37%) | 2.21 | (1.12-4.36) | 0.019 | 0.075 |
| LKT_s_Prevotella_stercorea | 4 | 35 | (56%) | 28 | (44%) | 2.21 | (1.12-4.36) | 0.02 | 0.077 |
| LKT_s_Pseudoflavonifractor_Unclassified | 373 | 38 | (60%) | 25 | (40%) | 2.17 | (1.1-4.26) | 0.022 | 0.08 |
| LKT_g_Streptococcus | 2650 | 53 | (84%) | 10 | (16%) | 2.46 | (1.11-5.45) | 0.022 | 0.081 |
| LKT_s_Faecalibacterium_Unclassified | 119 | 14 | (22%) | 49 | (78%) | 3.14 | (1.1-8.93) | 0.024 | 0.083 |
| LKT_s_Ruminococcus_albus | 54 | 44 | (70%) | 19 | (30%) | 2.18 | (1.09-4.36) | 0.024 | 0.083 |
| LKT_d_Eukaryota | 80 | 30 | (48%) | 33 | (52%) | 2.21 | (1.09-4.48) | 0.023 | 0.083 |
| LKT_s_Alistipes_timonensis | 68 | 43 | (68%) | 20 | (32%) | 2.16 | (1.09-4.29) | 0.024 | 0.084 |
| LKT_s_Oscillibacter_Unclassified | 2641 | 36 | (57%) | 27 | (43%) | 2.12 | (1.08-4.18) | 0.025 | 0.085 |
| LKT_s_Lactobacillus_salivarius | 1 | 13 | (21%) | 50 | (79%) | 3.56 | (1.09-11.7) | 0.025 | 0.085 |
| LKT_s_Intestinibacillus_massiliensis | 26 | 31 | (49%) | 32 | (51%) | 2.19 | (1.08-4.43) | 0.026 | 0.085 |
| LKT_s_Haemophilus_Unclassified | 1 | 20 | (32%) | 43 | (68%) | 2.48 | (1.08-5.7) | 0.028 | 0.087 |
| LKT_s_Acidaminococcus_fermentans | 4 | 24 | (38%) | 39 | (62%) | 2.29 | (1.07-4.92) | 0.029 | 0.089 |
| LKT_s_Streptococcus_salivarius | 2756 | 52 | (83%) | 11 | (17%) | 2.28 | (1.06-4.89) | 0.03 | 0.092 |
| LKT_s_Muribaculum_Unclassified | 1 | 14 | (22%) | 49 | (78%) | 2.99 | (1.05-8.53) | 0.031 | 0.095 |
| LKT_s_Clostridiales_bacterium | 8926 | 33 | (52%) | 30 | (48%) | 2.08 | (1.05-4.12) | 0.032 | 0.097 |
| LKT_g_Lactobacillus | 42 | 29 | (46%) | 34 | (54%) | 2.15 | (1.05-4.43) | 0.033 | 0.097 |
| LKT_s_Flavonifractor_plautii | 911 | 15 | (24%) | 48 | (76%) | 2.94 | (1.04-8.38) | 0.034 | 0.099 |
| LKT_g_Faecalibacterium | 191 | 12 | (19%) | 51 | (81%) | 3.34 | (1.02-11) | 0.035 | 0.101 |
| LKT_s_Bacteroides_massiliensis | 306 | 42 | (67%) | 21 | (33%) | 2.05 | (1.04-4.05) | 0.035 | 0.101 |
| LKT_s_Lactobacillus_paracasei | 1 | 24 | (38%) | 39 | (62%) | 2.22 | (1.03-4.78) | 0.036 | 0.101 |
| LKT_s_Ruminococcus_lactaris | 2914 | 47 | (75%) | 16 | (25%) | 2.1 | (1.04-4.26) | 0.036 | 0.101 |
| LKT_g_Subdoligranulum | 43 | 33 | (52%) | 30 | (48%) | 2.05 | (1.03-4.07) | 0.036 | 0.103 |
| LKT_s_Anaerotruncus_massiliensis | 171 | 37 | (59%) | 26 | (41%) | 2.02 | (1.03-3.97) | 0.038 | 0.105 |
| LKT_s_Ruminococcus_champanellensis | 6 | 12 | (19%) | 51 | (81%) | 3.25 | (0.993-10.7) | 0.039 | 0.109 |
| LKT_s_Enterobacter_hormaechei | 3 | 15 | (24%) | 48 | (76%) | 2.84 | (0.999-8.08) | 0.041 | 0.11 |
| LKT_g_Clostridium | 459 | 12 | (19%) | 51 | (81%) | 3.21 | (0.981-10.5) | 0.042 | 0.112 |
| LKT_s_Cyclospora_cayetanensis | 5392 | 29 | (46%) | 34 | (54%) | 2.05 | (1.01-4.15) | 0.042 | 0.112 |
| LKT_s_Anaeromassilibacillus_senegalensis | 12 | 12 | (19%) | 51 | (81%) | 3.2 | (0.978-10.5) | 0.042 | 0.112 |
| LKT_s_Bacteroides_ovatus | 1987 | 20 | (32%) | 43 | (68%) | 2.3 | (1-5.3) | 0.043 | 0.113 |
| LKT_s_Butyricimonas_virosa | 8 | 20 | (32%) | 43 | (68%) | 2.3 | (1-5.31) | 0.043 | 0.113 |
| LKT_s_Ruthenibacterium_lactatiformans | 322 | 22 | (35%) | 41 | (65%) | 2.2 | (0.994-4.87) | 0.046 | 0.115 |
| LKT_s_Bacteroides_finegoldii | 142 | 20 | (32%) | 43 | (68%) | 2.28 | (0.992-5.25) | 0.046 | 0.115 |
| LKT_s_Alistipes_putredinis | 16450 | 47 | (75%) | 16 | (25%) | 2.05 | (0.996-4.21) | 0.046 | 0.116 |
| LKT_s_Butyricicoccus_Unclassified | 1453 | 25 | (40%) | 38 | (60%) | 2.08 | (0.991-4.35) | 0.048 | 0.116 |
| LKT_s_Bacteroides_cellulosilyticus | 4876 | 37 | (59%) | 26 | (41%) | 1.95 | (0.994-3.83) | 0.048 | 0.116 |
| LKT_s_Bacteroides_coprocola | 29 | 30 | (48%) | 33 | (52%) | 1.99 | (0.993-3.97) | 0.048 | 0.116 |
| LKT_s_Intestinimonas_timonensis | 280 | 32 | (51%) | 31 | (49%) | 1.96 | (0.99-3.9) | 0.049 | 0.117 |
| LKT_s_Streptococcus_parasanguinis | 449 | 49 | (78%) | 14 | (22%) | 2.07 | (0.988-4.34) | 0.049 | 0.117 |
| LKT_s_Victivallis_vadensis | 1 | 15 | (24%) | 48 | (76%) | 2.51 | (0.968-6.49) | 0.05 | 0.118 |
| LKT_s_Sutterella_wadsworthensis | 3 | 23 | (37%) | 40 | (63%) | 2.11 | (0.984-4.55) | 0.05 | 0.118 |
| LKT_s_Clostridium_botulinum | 22 | 47 | (75%) | 16 | (25%) | 2 | (0.984-4.07) | 0.051 | 0.118 |
| LKT_s_Coprococcus_eutactus | 246 | 41 | (65%) | 22 | (35%) | 1.94 | (0.985-3.83) | 0.051 | 0.118 |
| LKT_s_Anaerotignum_lactatifermentans | 283 | 36 | (57%) | 27 | (43%) | 1.94 | (0.984-3.81) | 0.051 | 0.118 |
| LKT_s_Duncaniella_Unclassified | 1 | 31 | (49%) | 32 | (51%) | 1.96 | (0.978-3.93) | 0.053 | 0.12 |
| LKT_g_Escherichia | 97 | 43 | (68%) | 20 | (32%) | 1.95 | (0.978-3.87) | 0.053 | 0.12 |
| LKT_s_Acidaminococcus_Unclassified | 8 | 53 | (84%) | 10 | (16%) | 2.15 | (0.969-4.75) | 0.054 | 0.12 |
| LKT_s_Dysosmobacter_welbionis | 110 | 13 | (21%) | 50 | (79%) | 2.67 | (0.94-7.6) | 0.055 | 0.122 |
| LKT_g_Butyricimonas | 3 | 27 | (43%) | 36 | (57%) | 1.99 | (0.969-4.1) | 0.056 | 0.123 |
| LKT_s_Bacteroides_intestinalis | 57 | 11 | (17%) | 52 | (83%) | 3 | (0.917-9.84) | 0.056 | 0.123 |
| LKT_s_Bilophila_wadsworthia | 19 | 13 | (21%) | 50 | (79%) | 2.62 | (0.921-7.46) | 0.061 | 0.13 |
| LKT_f_Enterobacteriaceae | 1289 | 38 | (60%) | 25 | (40%) | 1.86 | (0.949-3.66) | 0.066 | 0.137 |
| LKT_s_Desulfovibrio_fairfieldensis | 1 | 19 | (30%) | 44 | (70%) | 2.14 | (0.929-4.92) | 0.067 | 0.139 |
| LKT_s_Lactobacillus_gasseri | 2 | 29 | (46%) | 34 | (54%) | 1.9 | (0.94-3.85) | 0.069 | 0.141 |
| LKT_s_Agathobaculum_butyriciproducens | 100 | 19 | (30%) | 44 | (70%) | 2.12 | (0.921-4.88) | 0.071 | 0.143 |
| LKT_s_Klebsiella_pneumoniae | 24 | 23 | (37%) | 40 | (63%) | 2.03 | (0.916-4.5) | 0.075 | 0.148 |
| LKT_s_Faecalitalea_cylindroides | 60 | 50 | (79%) | 13 | (21%) | 1.97 | (0.915-4.22) | 0.077 | 0.149 |
| LKT_s_Porphyromonas_asaccharolytica | 3 | 44 | (70%) | 19 | (30%) | 1.85 | (0.922-3.72) | 0.079 | 0.149 |
| LKT_s_Desulfovibrio_desulfuricans | 3 | 38 | (60%) | 25 | (40%) | 1.81 | (0.92-3.56) | 0.082 | 0.149 |
| LKT_s_Harryflintia_acetispora | 154 | 32 | (51%) | 31 | (49%) | 1.83 | (0.92-3.62) | 0.08 | 0.149 |
| LKT_s_Butyricicoccus_pullicaecorum | 54 | 19 | (30%) | 44 | (70%) | 2.08 | (0.905-4.78) | 0.078 | 0.149 |
| LKT_s_Lactobacillus_paragasseri | 6 | 51 | (81%) | 12 | (19%) | 1.96 | (0.914-4.21) | 0.078 | 0.149 |
| LKT_s_Alistipes_indistinctus | 3 | 20 | (32%) | 43 | (68%) | 2 | (0.905-4.43) | 0.081 | 0.149 |
| LKT_s_Akkermansia_Unclassified | 3 | 25 | (40%) | 38 | (60%) | 1.91 | (0.913-4.01) | 0.08 | 0.149 |
| LKT_s_Cryptobacterium_curtum | 4 | 48 | (76%) | 15 | (24%) | 1.9 | (0.919-3.93) | 0.078 | 0.149 |
| LKT_s_Escherichia_coli | 10870 | 49 | (78%) | 14 | (22%) | 1.93 | (0.919-4.04) | 0.077 | 0.149 |
| LKT_s_Bacteroides_vulgatus | 7610 | 17 | (27%) | 46 | (73%) | 2.16 | (0.894-5.24) | 0.08 | 0.149 |

TABLE 6
Cox Regression Models (Overall Survival and Progression-
Free Survival) for immune related Adverse Effects
(Pittsburgh Early Sample Cohort)

| | Overall Survival | Progression Free Survival |
|---|---|---|
| | Univariate HR, 95% | Univariate HR, 95% |
| irAE | CI, p-value | CI, p-value |
| Grade 1-4, reported vs | 0.504 (0.239-1.06), | 0.47 (0.237-0.929), |
| non-reported | P = 0.067 | P = 0.026 |
| Grade 1-2 (highest) vs | 0.51 (0.217-1.2), | 0.35 (0.153-0.801), |
| no AE-reported | P = 0.115 | P = 0.010 |
| Grade 3-4 (highest) vs | 0.504 (0.19-1.34), | 0.686 (0.302-1.56), |
| no AE-reported | P = 0.161 | P = 0.366 |

TABLE 7
LDA scores of differentially abundant taxa from five
separate anti-PD-1-treated melanoma patient cohorts

| | | | LDA | |
|---|---|---|---|---|
| Taxa | Abundance | Class | score | p-value |
| k_Bacteria; p_Actinobacteria; | 1.44 | Non_Responder | 2.072 | 0.015 |
| c_Actinobacteria; o_Corynebacteriales; | | | | |
| f_Mycobacteriaceae; g_Unclassified | | | | |
| k_Bacteria; p_Actinobacteria; | 1.44 | Non_Responder | 2.072 | 0.015 |
| c_Actinobacteria; o_Corynebacteriales; | | | | |
| f_Mycobacteriaceae; g_Unclassified; | | | | |
| LKT_f_Mycobacteriaceae | | | | |
| k_Bacteria; p_Bacteroidetes; | 4.273 | Non_Responder | 3.716 | 0.013 |
| c_Bacteroidia; o_Bacteroidales; | | | | |
| f_Bacteroidaceae; g_Bacteroides; | | | | |
| LKT_s_Bacteroides_{—}cellulosilyticus | | | | |
| k_Bacteria; p_Bacteroidetes; | 4.216 | Non_Responder | 3.425 | 0.04 |
| c_Bacteroidia; o_Bacteroidales; | | | | |
| f_Bacteroidaceae; g_Bacteroides; | | | | |
| LKT_s_Bacteroides_{—}fragilis | | | | |
| k_Bacteria; p_Bacteroidetes; | 4.274 | Non_Responder | 3.422 | 0.031 |
| c_Bacteroidia; o_Bacteroidales; | | | | |
| f_Bacteroidaceae; g_Bacteroides; | | | | |
| LKT_s_Bacteroides_{—}intestinalis | | | | |
| k_Bacteria; p_Bacteroidetes; | 4.095 | Non_Responder | 3.265 | 0.014 |
| c_Bacteroidia; o_Bacteroidales; | | | | |
| f_Odoribacteraceae | | | | |
| k_Bacteria; p_Bacteroidetes; | 3.4 | Non_Responder | 2.566 | 0.044 |
| c_Bacteroidia; o_Bacteroidales; | | | | |
| f_Odoribacteraceae; g_Butyricimonas | | | | |
| k_Bacteria; p_Bacteroidetes; | 3.054 | Non_Responder | 2.263 | 0.047 |
| c_Bacteroidia; o_Bacteroidales; | | | | |
| f_Odoribacteraceae; g_Butyricimonas; | | | | |
| LKT_s_Butyricimonas_{—}virosa | | | | |
| k_Bacteria; p_Bacteroidetes; | 3.964 | Non_Responder | 3.183 | 0.015 |
| c_Bacteroidia; o_Bacteroidales; | | | | |
| f_Odoribacteraceae; g_Odoribacter | | | | |
| k_Bacteria; p_Bacteroidetes; | 2.963 | Non_Responder | 2.374 | 0.047 |
| c_Bacteroidia; o_Bacteroidales; | | | | |
| f_Odoribacteraceae; g_Odoribacter; | | | | |
| LKT_g_Odoribacter | | | | |
| k_Bacteria; p_Bacteroidetes; | 3.132 | Non_Responder | 2.722 | 0.009 |
| c_Bacteroidia; o_Bacteroidales; | | | | |
| f_Odoribacteraceae; g_Odoribacter; | | | | |
| LKT_s_Odoribacter_{—}laneus | | | | |
| k_Bacteria; p_Bacteroidetes; | 3.822 | Non_Responder | 3.069 | 0.048 |
| c_Bacteroidia; o_Bacteroidales; | | | | |
| f_Odoribacteraceae; g_Odoribacter; | | | | |
| LKT_s_Odoribacter_{—}splanchnicus | | | | |
| k_Bacteria; p_Bacteroidetes; | 4.454 | Non_Responder | 3.822 | 0.003 |
| c_Bacteroidia; o_Bacteroidales; | | | | |
| f_Prevotellaceae | | | | |
| k_Bacteria; p_Bacteroidetes; | 4.397 | Non_Responder | 3.781 | 0.002 |
| c_Bacteroidia; o_Bacteroidales; | | | | |
| f_Prevotellaceae; g_Prevotella | | | | |
| k_Bacteria; p_Bacteroidetes; | 2.899 | Non_Responder | 2.443 | 0.031 |
| c_Bacteroidia; o_Bacteroidales; | | | | |
| f_Prevotellaceae; g_Prevotella; | | | | |
| LKT_g_Prevotella | | | | |
| k_Bacteria; p_Bacteroidetes; | 4.044 | Non_Responder | 3.457 | 0 |
| c_Bacteroidia; o_Bacteroidales; | | | | |
| f_Prevotellaceae; g_Prevotella; | | | | |
| LKT_s_Prevotella_{—}copri | | | | |
| k_Bacteria; p_Bacteroidetes; | 2.876 | Non_Responder | 2.361 | 0.001 |
| c_Bacteroidia; o_Bacteroidales; | | | | |
| f_Prevotellaceae; g_Prevotella; | | | | |
| LKT_s_Prevotella_{—}lascolaii | | | | |
| k_Bacteria; p_Bacteroidetes; | 2.561 | Non_Responder | 2.084 | 0.003 |
| c_Bacteroidia; o_Bacteroidales; | | | | |
| f_Prevotellaceae; g_Prevotella; | | | | |
| LKT_s_Prevotella_{—}ruminicola | | | | |
| k_Bacteria; p_Bacteroidetes; | 3.27 | Non_Responder | 2.764 | 0 |
| c_Bacteroidia; o_Bacteroidales; | | | | |
| f_Prevotellaceae; g_Prevotella; | | | | |
| LKT_s_Prevotella_{—}stercorea | | | | |
| k_Bacteria; p_Bacteroidetes; | 3.927 | Non_Responder | 3.404 | 0 |
| c_Bacteroidia; o_Bacteroidales; | | | | |
| f_Prevotellaceae; g_Prevotella; | | | | |
| LKT_s_Prevotella_Unclassified | | | | |
| k_Bacteria; p_Bacteroidetes; | 2.593 | Non_Responder | 2.172 | 0 |
| c_Bacteroidia; o_Bacteroidales; | | | | |
| f_Prevotellaceae; g_Prevotellamassilia | | | | |
| k_Bacteria; p_Bacteroidetes; | 2.593 | Non_Responder | 2.172 | 0 |
| c_Bacteroidia; o_Bacteroidales; | | | | |
| f_Prevotellaceae; g_Prevotellamassilia; | | | | |
| LKT_s_Prevotellamassilia_{—}timonensis | | | | |
| k_Bacteria; p_Bacteroidetes; | 4.794 | Non_Responder | 4.031 | 0.043 |
| c_Bacteroidia; o_Bacteroidales; | | | | |
| f_Rikenellaceae | | | | |
| k_Bacteria; p_Bacteroidetes; | 4.791 | Non_Responder | 4.038 | 0.037 |
| c_Bacteroidia; o_Bacteroidales; | | | | |
| f_Rikenellaceae; g_Alistipes | | | | |
| k_Bacteria; p_Bacteroidetes; | 3.208 | Non_Responder | 2.382 | 0.021 |
| c_Bacteroidia; o_Bacteroidales; | | | | |
| f_Rikenellaceae; g_Alistipes; | | | | |
| LKT_g_Alistipes | | | | |
| k_Bacteria; p_Bacteroidetes; | 3.572 | Non_Responder | 2.942 | 0.019 |
| c_Bacteroidia; o_Bacteroidales; | | | | |
| f_Rikenellaceae; g_Alistipes; | | | | |
| LKT_s_Alistipes_{—}obesi | | | | |
| k_Bacteria; p_Bacteroidetes; | 3.659 | Non_Responder | 2.98 | 0.034 |
| c_Bacteroidia; o_Bacteroidales; | | | | |
| f_Rikenellaceae; g_Alistipes; | | | | |
| LKT_s_Alistipes_{—}senegalensis | | | | |
| k_Bacteria; p_Bacteroidetes; | 3.183 | Non_Responder | 2.554 | 0.005 |
| c_Bacteroidia; o_Bacteroidales; | | | | |
| f_Rikenellaceae; g_Alistipes; | | | | |
| LKT_s_Alistipes_{—}timonensis | | | | |
| k_Bacteria; p_Bacteroidetes; | 4.123 | Non_Responder | 3.441 | 0.028 |
| c_Bacteroidia; o_Bacteroidales; | | | | |
| f_Rikenellaceae; g_Alistipes; | | | | |
| LKT_s_Alistipes_Unclassified | | | | |
| k_Bacteria; p_Firmicutes; | 0.745 | Non_Responder | 2.025 | 0.027 |
| c_Bacilli; o_Bacillales; | | | | |
| f_Planococcaceae; g_Rummeliibacillus | | | | |
| k_Bacteria; p_Firmicutes; | 0.745 | Non_Responder | 2.025 | 0.027 |
| c_Bacilli; o_Bacillales; | | | | |
| f_Planococcaceae; g_Rummeliibacillus; | | | | |
| LKT_s_Rummeliibacillus_{—}pycnus | | | | |
| k_Bacteria; p_Firmicutes; | 2.534 | Non_Responder | 2.362 | 0.001 |
| c_Bacilli; o_Lactobacillies; | | | | |
| f_Lactobacilluseae; g_Lactobacillus; | | | | |
| LKT_s_Lactobacillus_{—}animalis | | | | |
| k_Bacteria; p_Firmicutes; | 3.448 | Non_Responder | 2.193 | 0.038 |
| c_Clostridia; o_Clostridiales; | | | | |
| f_Oscillospiraceae; g_Unclassified | | | | |
| k_Bacteria; p_Firmicutes; | 3.448 | Non_Responder | 2.193 | 0.038 |
| c_Clostridia; o_Clostridiales; | | | | |
| f_Oscillospiraceae; g_Unclassified; | | | | |
| LKT_f_Oscillospiraceae | | | | |
| k_Bacteria; p_Firmicutes; | 3.537 | Non_Responder | 2.571 | 0.027 |
| c_Clostridia; o_Clostridiales; | | | | |
| f_Ruminococcaceae; g_Unclassified | | | | |
| k_Bacteria; p_Firmicutes; | 3.537 | Non_Responder | 2.571 | 0.027 |
| c_Clostridia; o_Clostridiales; | | | | |
| f_Ruminococcaceae; g_Unclassified; | | | | |
| LKT_f_Ruminococcaceae | | | | |
| k_Bacteria; p_Proteobacteria; | 3.285 | Non_Responder | 2.511 | 0.038 |
| c_Deltaproteobacteria; o_Desulfovibrionales; | | | | |
| f_Desulfovibrionaceae; g_Desulfovibrio | | | | |
| k_Bacteria; p_Proteobacteria; | 2.73 | Non_Responder | 2.181 | 0.034 |
| c_Deltaproteobacteria; o_Desulfovibrionales; | | | | |
| f_Desulfovibrionaceae; g_Desulfovibrio; | | | | |
| LKT_s_Desulfovibrio_{—}fairfieldensis | | | | |
| k_Bacteria; p_Proteobacteria; | 1.304 | Non_Responder | 2.022 | 0.047 |
| c_Gammaproteobacteria; o_Aeromonadales; | | | | |
| f_Aeromonadaceae; g_Aeromonas; | | | | |
| LKT_s_Aeromonas_{—}veronii | | | | |
| k_Bacteria; p_Verrucomicrobia | 4.217 | Non_Responder | 3.302 | 0.005 |
| k_Bacteria; p_Verrucomicrobia; | 4.217 | Non_Responder | 3.302 | 0.005 |
| c_Verrucomicrobiae | | | | |
| k_Bacteria; p_Verrucomicrobia; | 4.217 | Non_Responder | 3.302 | 0.005 |
| c_Verrucomicrobiae; o_Verrucomicrobiales | | | | |
| k_Bacteria; p_Verrucomicrobia; | 4.217 | Non_Responder | 3.302 | 0.005 |
| c_Verrucomicrobiae; o_Verrucomicrobiales; | | | | |
| f_Akkermansiaceae | | | | |
| k_Bacteria; p_Verrucomicrobia; | 4.217 | Non_Responder | 3.302 | 0.005 |
| c_Verrucomicrobiae; o_Verrucomicrobiales; | | | | |
| f_Akkermansiaceae; g_Akkermansia | | | | |
| k_Bacteria; p_Verrucomicrobia; | 3.263 | Non_Responder | 2.719 | 0.045 |
| c_Verrucomicrobiae; o_Verrucomicrobiales; | | | | |
| f_Akkermansiaceae; g_Akkermansia; | | | | |
| LKT_g_Akkermansia | | | | |
| k_Bacteria; p_Verrucomicrobia; | 4.166 | Non_Responder | 3.269 | 0.005 |
| c_Verrucomicrobiae; o_Verrucomicrobiales; | | | | |
| f_Akkermansiaceae; g_Akkermansia; | | | | |
| LKT_s_Akkermansia_{—}muciniphila | | | | |
| k_Bacteria; p_Actinobacteria; | 2.739 | Responder | 2.038 | 0.008 |
| c_Actinobacteria; o_Actinomycetales | | | | |
| k_Bacteria; p_Actinobacteria; | 2.739 | Responder | 2.038 | 0.008 |
| c_Actinobacteria; o_Actinomycetales; | | | | |
| f_Actinomycetaceae | | | | |
| k_Bacteria; p_Actinobacteria; | 0.195 | Responder | 2.654 | 0 |
| c_Actinobacteria; o_Actinomycetales; | | | | |
| f_Actinomycetaceae; g_Unclassified | | | | |
| k_Bacteria; p_Actinobacteria; | 0.195 | Responder | 2.654 | 0 |
| c_Actinobacteria; o_Actinomycetales; | | | | |
| f_Actinomycetaceae; g_Unclassified; | | | | |
| LKT_f_Actinomycetaceae | | | | |
| k_Bacteria; p_Actinobacteria; | 3.033 | Responder | 2.585 | 0.03 |
| c_Actinobacteria; o_Bifidobacteriales; | | | | |
| f_Bifidobacteriaceae; g_Bifidobacterium; | | | | |
| LKT_s_Bifidobacterium_{—}pseudocatenulatum | | | | |
| k_Bacteria; p_Actinobacteria; | 0.991 | Responder | 2.093 | 0.001 |
| c_Actinobacteria; o_Micrococcales; | | | | |
| f_Promicromonosporaceae | | | | |
| k_Bacteria; p_Actinobacteria; | 0.991 | Responder | 2.093 | 0.001 |
| c_Actinobacteria; o_Micrococcales; | | | | |
| f_Promicromonosporaceae; g_Xylanimonas | | | | |
| k_Bacteria; p_Actinobacteria; | 0.991 | Responder | 2.093 | 0.001 |
| c_Actinobacteria; o_Micrococcales; | | | | |
| f_Promicromonosporaceae; g_Xylanimonas; | | | | |
| LKT_s_Xylanimonas_{—}cellulosilytica | | | | |
| k_Bacteria; p_Actinobacteria; | 0.883 | Responder | 2.147 | 0.002 |
| c_Actinobacteria; o_Micrococcales; | | | | |
| f_Sanguibacteraceae | | | | |
| k_Bacteria; p_Actinobacteria; | 0.883 | Responder | 2.147 | 0.002 |
| c_Actinobacteria; o_Micrococcales; | | | | |
| f_Sanguibacteraceae; g_Sanguibacter | | | | |
| k_Bacteria; p_Actinobacteria; | 0.883 | Responder | 2.147 | 0.002 |
| c_Actinobacteria; o_Micrococcales; | | | | |
| f_Sanguibacteraceae; g_Sanguibacter; | | | | |
| LKT_s_Sanguibacter_{—}keddieii | | | | |
| k_Bacteria; p_Bacteroidetes; | 2.996 | Responder | 2.529 | 0.028 |
| c_Bacteroidia; o_Bacteroidales; | | | | |
| f_Prevotellaceae; g_Prevotella; | | | | |
| LKT_s_Prevotella_{—}buccae | | | | |
| k_Bacteria; p_Bacteroidetes; | 2.619 | Responder | 2.118 | 0.007 |
| c_Bacteroidia; o_Bacteroidales; | | | | |
| f_Prevotellaceae; g_Prevotella; | | | | |
| LKT_s_Prevotella_{—}denticola | | | | |
| k_Bacteria; p_Firmicutes | 5.647 | Responder | 4.557 | 0.001 |
| k_Bacteria; p_Firmicutes; | 0.803 | Responder | 2.008 | 0.022 |
| c_Bacilli; o_Lactobacillies; | | | | |
| f_Lactobacillus; g_Lactobacillus; | | | | |
| LKT_s_Lactobacillus_{—}acidophilus | | | | |
| k_Bacteria; p_Firmicutes; | 2.569 | Responder | 2.006 | 0.037 |
| c_Bacilli; o_Lactobacillies; | | | | |
| f_Streptococcaceae; g_Streptococcus; | | | | |
| LKT_g_Streptococcus | | | | |
| k_Bacteria; p_Firmicutes; | 5.602 | Responder | 4.53 | 0.002 |
| c_Clostridia | | | | |
| k_Bacteria; p_Firmicutes; | 5.602 | Responder | 4.53 | 0.002 |
| c_Clostridia; o_Clostridiales | | | | |
| k_Bacteria; p_Firmicutes; | 3.548 | Responder | 2.533 | 0.049 |
| c_Clostridia; o_Clostridiales; | | | | |
| f_Clostridiaceae; g_Butyricicoccus | | | | |
| k_Bacteria; p_Firmicutes; | 3.593 | Responder | 2.623 | 0.027 |
| c_Clostridia; o_Clostridiales; | | | | |
| f_Clostridiaceae; g_Clostridium; | | | | |
| LKT_g_Clostridium | | | | |
| k_Bacteria; p_Firmicutes; | 1.053 | Responder | 2.132 | 0.034 |
| c_Clostridia; o_Clostridiales; | | | | |
| f_Clostridiaceae; g_Clostridium; | | | | |
| LKT_s_Clostridium_{—}cadaveris | | | | |
| k_Bacteria; p_Firmicutes; | 1.084 | Responder | 2.072 | 0.02 |
| c_Clostridia; o_Clostridiales; | | | | |
| f_Clostridiaceae; g_Clostridium; | | | | |
| LKT_s_Clostridium_{—}kluyveri | | | | |
| k_Bacteria; p_Firmicutes; | 4.326 | Responder | 3.321 | 0.041 |
| c_Clostridia; o_Clostridiales; | | | | |
| f_Clostridiaceae; g_Clostridium; | | | | |
| LKT_s_Clostridium_Unclassified | | | | |
| k_Bacteria; p_Firmicutes; | 2.925 | Responder | 2.003 | 0.001 |
| c_Clostridia; o_Clostridiales; | | | | |
| f_Clostridiaceae; g_Missing | | | | |
| k_Bacteria; p_Firmicutes; | 2.925 | Responder | 2.003 | 0.001 |
| c_Clostridia; o_Clostridiales; | | | | |
| f_Clostridiaceae; g_Missing; | | | | |
| LKT_s_Clostridiaceae_{—}bacterium | | | | |
| k_Bacteria; p_Firmicutes; | 3.155 | Responder | 2.087 | 0.036 |
| c_Clostridia; o_Clostridiales; | | | | |
| f_Eubacteriaceae; g_Eubacterium; | | | | |
| LKT_s_Eubacterium_{—}ventriosum | | | | |
| k_Bacteria; p_Firmicutes; | 5.24 | Responder | 4.295 | 0.001 |
| c_Clostridia; o_Clostridiales; | | | | |
| f_Lachnospiraceae | | | | |
| k_Bacteria; p_Firmicutes; | 3.493 | Responder | 2.726 | 0.013 |
| c_Clostridia; o_Clostridiales; | | | | |
| f_Lachnospiraceae; g_Anaerostipes | | | | |
| k_Bacteria; p_Firmicutes; | 3.429 | Responder | 2.715 | 0.039 |
| c_Clostridia; o_Clostridiales; | | | | |
| f_Lachnospiraceae; g_Anaerostipes; | | | | |
| LKT_s_Anaerostipes_{—}hadrus | | | | |
| k_Bacteria; p_Firmicutes; | 4.667 | Responder | 3.738 | 0.008 |
| c_Clostridia; o_Clostridiales; | | | | |
| f_Lachnospiraceae; g_Blautia | | | | |
| k_Bacteria; p_Firmicutes; | 3.708 | Responder | 2.821 | 0.006 |
| c_Clostridia; o_Clostridiales; | | | | |
| f_Lachnospiraceae; g_Blautia; | | | | |
| LKT_g_Blautia | | | | |
| k_Bacteria; p_Firmicutes; | 3.67 | Responder | 2.856 | 0.005 |
| c_Clostridia; o_Clostridiales; | | | | |
| f_Lachnospiraceae; g_Blautia; | | | | |
| LKT_s_Blautia_{—}hansenii | | | | |
| k_Bacteria; p_Firmicutes; | 2.95 | Responder | 2.287 | 0.02 |
| c_Clostridia; o_Clostridiales; | | | | |
| f_Lachnospiraceae; g_Blautia; | | | | |
| LKT_s_Blautia_{—}massiliensis | | | | |
| k_Bacteria; p_Firmicutes; | 3.96 | Responder | 2.775 | 0.042 |
| c_Clostridia; o_Clostridiales; | | | | |
| f_Lachnospiraceae; g_Blautia; | | | | |
| LKT_s_Blautia_{—}obeum | | | | |
| k_Bacteria; p_Firmicutes; | 3.231 | Responder | 2.508 | 0.009 |
| c_Clostridia; o_Clostridiales; | | | | |
| f_Lachnospiraceae; g_Blautia; | | | | |
| LKT_s_Blautia_{—}producta | | | | |
| k_Bacteria; p_Firmicutes; | 4.072 | Responder | 3.123 | 0.004 |
| c_Clostridia; o_Clostridiales; | | | | |
| f_Lachnospiraceae; g_Blautia; | | | | |
| LKT_s_Blautia_Unclassified | | | | |
| k_Bacteria; p_Firmicutes; | 3.361 | Responder | 2.587 | 0.007 |
| c_Clostridia; o_Clostridiales; | | | | |
| f_Lachnospiraceae; g_Blautia; | | | | |
| LKT_s_Blautia_{—}wexlerae | | | | |
| k_Bacteria; p_Firmicutes; | 3.751 | Responder | 3.091 | 0.007 |
| c_Clostridia; o_Clostridiales; | | | | |
| f_Lachnospiraceae; g_Blautia; | | | | |
| LKT_s_Ruminococcus_{—}gnavus | | | | |
| k_Bacteria; p_Firmicutes; | 3.581 | Responder | 2.994 | 0.032 |
| c_Clostridia; o_Clostridiales; | | | | |
| f_Lachnospiraceae; g_Butyrivibrio | | | | |
| k_Bacteria; p_Firmicutes; | 3.343 | Responder | 2.947 | 0 |
| c_Clostridia; o_Clostridiales; | | | | |
| f_Lachnospiraceae; g_Butyrivibrio; | | | | |
| LKT_s_Butyrivibrio_{—}crossotus | | | | |
| k_Bacteria; p_Firmicutes; | 1.542 | Responder | 2.169 | 0.01 |
| c_Clostridia; o_Clostridiales; | | | | |
| f_Lachnospiraceae; g_Butyrivibrio; | | | | |
| LKT_s_Butyrivibrio_{—}fibrisolvens | | | | |
| k_Bacteria; p_Firmicutes; | 1.696 | Responder | 2.017 | 0.005 |
| c_Clostridia; o_Clostridiales; | | | | |
| f_Lachnospiraceae; g_Butyrivibrio; | | | | |
| LKT_s_Butyrivibrio_{—}proteoclasticus | | | | |
| k_Bacteria; p_Firmicutes; | 2.835 | Responder | 2.087 | 0 |
| c_Clostridia; o_Clostridiales; | | | | |
| f_Lachnospiraceae; g_Coprococcus; | | | | |
| LKT_s_Coprococcus_Unclassified | | | | |
| k_Bacteria; p_Firmicutes; | 3.411 | Responder | 2.65 | 0.003 |
| c_Clostridia; o_Clostridiales; | | | | |
| f_Lachnospiraceae; g_Dorea; | | | | |
| LKT_s_Dorea_{—}formicigenerans | | | | |
| k_Bacteria; p_Firmicutes; | 3.048 | Responder | 2.116 | 0.008 |
| c_Clostridia; o_Clostridiales; | | | | |
| f_Lachnospiraceae; g_Dorea; | | | | |
| LKT_s_Dorea_Unclassified | | | | |
| k_Bacteria; p_Firmicutes; | 3.988 | Responder | 2.893 | 0.037 |
| c_Clostridia; o_Clostridiales; | | | | |
| f_Lachnospiraceae; g_Lachnoclostridium | | | | |
| k_Bacteria; p_Firmicutes; | 1.31 | Responder | 2.17 | 0.013 |
| c_Clostridia; o_Clostridiales; | | | | |
| f_Lachnospiraceae; g_Lachnoclostridium; | | | | |
| LKT_s_Lachnoclostridium_{—}phytofermentans | | | | |
| k_Bacteria; p_Firmicutes; | 4.655 | Responder | 3.843 | 0.001 |
| c_Clostridia; o_Clostridiales; | | | | |
| f_Lachnospiraceae; g_Missing | | | | |
| k_Bacteria; p_Firmicutes; | 4.455 | Responder | 3.717 | 0.001 |
| c_Clostridia; o_Clostridiales; | | | | |
| f_Lachnospiraceae; g_Missing; | | | | |
| LKT_s_Eubacterium_{—}rectale | | | | |
| k_Bacteria; p_Firmicutes; | 4.222 | Responder | 3.244 | 0.04 |
| c_Clostridia; o_Clostridiales; | | | | |
| f_Lachnospiraceae; g_Missing; | | | | |
| LKT_s_Lachnospiraceae_{—}bacterium | | | | |
| k_Bacteria; p_Firmicutes; | 4.424 | Responder | 3.374 | 0.009 |
| c_Clostridia; o_Clostridiales; | | | | |
| f_Lachnospiraceae; g_Roseburia | | | | |
| k_Bacteria; p_Firmicutes; | 3.209 | Responder | 2.484 | 0 |
| c_Clostridia; o_Clostridiales; | | | | |
| f_Lachnospiraceae; g_Roseburia; | | | | |
| LKT_g_Roseburia | | | | |
| k_Bacteria; p_Firmicutes; | 3.269 | Responder | 2.466 | 0.028 |
| c_Clostridia; o_Clostridiales; | | | | |
| f_Lachnospiraceae; g_Roseburia; | | | | |
| LKT_s_Roseburia_{—}faecis | | | | |
| k_Bacteria; p_Firmicutes; | 3.657 | Responder | 2.78 | 0.022 |
| c_Clostridia; o_Clostridiales; | | | | |
| f_Lachnospiraceae; g_Roseburia; | | | | |
| LKT_s_Roseburia_{—}hominis | | | | |
| k_Bacteria; p_Firmicutes; | 3.777 | Responder | 3.017 | 0.001 |
| c_Clostridia; o_Clostridiales; | | | | |
| f_Lachnospiraceae; g_Roseburia; | | | | |
| LKT_s_Roseburia_Unclassified | | | | |
| k_Bacteria; p_Firmicutes; | 3.358 | Responder | 2.799 | 0.007 |
| c_Clostridia; o_Clostridiales; | | | | |
| f_Lachnospiraceae; g_Sellimonas | | | | |
| k_Bacteria; p_Firmicutes; | 3.358 | Responder | 2.799 | 0.007 |
| c_Clostridia; o_Clostridiales; | | | | |
| f_Lachnospiraceae; g_Sellimonas; | | | | |
| LKT_s_Sellimonas_{—}intestinalis | | | | |
| k_Bacteria; p_Firmicutes; | 1.572 | Responder | 2.011 | 0.002 |
| c_Clostridia; o_Clostridiales; | | | | |
| f_Peptococcaceae; g_Desulfotomaculum; | | | | |
| LKT_s_Desulfotomaculum_Unclassified | | | | |
| k_Bacteria; p_Firmicutes; | 3.197 | Responder | 2.055 | 0.003 |
| c_Clostridia; o_Clostridiales; | | | | |
| f_Peptostreptococcaceae; g_Clostridioides | | | | |
| k_Bacteria; p_Firmicutes; | 3.197 | Responder | 2.055 | 0.003 |
| c_Clostridia; o_Clostridiales; | | | | |
| f_Peptostreptococcaceae; g_Clostridioides; | | | | |
| LKT_s_Clostridioides_{—}difficile | | | | |
| k_Bacteria; p_Firmicutes; | 5.13 | Responder | 4.033 | 0.046 |
| c_Clostridia; o_Clostridiales; | | | | |
| f_Ruminococcaceae | | | | |
| k_Bacteria; p_Firmicutes; | 3.928 | Responder | 3.014 | 0.044 |
| c_Erysipelotrichia | | | | |
| k_Bacteria; p_Firmicutes; | 3.928 | Responder | 3.014 | 0.044 |
| c_Erysipelotrichia; o_Erysipelotrichales | | | | |
| k_Bacteria; p_Firmicutes; | 3.928 | Responder | 3.014 | 0.044 |
| c_Erysipelotrichia; o_Erysipelotrichales; | | | | |
| f_Erysipelotrichaceae | | | | |
| k_Bacteria; p_Firmicutes; | 2.577 | Responder | 2.147 | 0.035 |
| c_Erysipelotrichia; o_Erysipelotrichales; | | | | |
| f_Erysipelotrichaceae; g_Catenibacterium | | | | |
| k_Bacteria; p_Firmicutes; | 2.577 | Responder | 2.147 | 0.035 |
| c_Erysipelotrichia; o_Erysipelotrichales; | | | | |
| f_Erysipelotrichaceae; g_Catenibacterium; | | | | |
| LKT_g_Catenibacterium | | | | |
| k_Bacteria; p_Firmicutes; | 2.975 | Responder | 2.32 | 0.012 |
| c_Erysipelotrichia; o_Erysipelotrichales; | | | | |
| f_Erysipelotrichaceae; g_Coprobacillus; | | | | |
| LKT_g_Coprobacillus | | | | |
| k_Bacteria; p_Firmicutes; | 3.071 | Responder | 2.438 | 0.007 |
| c_Erysipelotrichia; o_Erysipelotrichales; | | | | |
| f_Erysipelotrichaceae; g_Coprobacillus; | | | | |
| LKT_s_Coprobacillus_Unclassified | | | | |
| k_Bacteria; p_Firmicutes; | 1.309 | Responder | 2.452 | 0.002 |
| c_Erysipelotrichia; o_Erysipelotrichales; | | | | |
| f_Erysipelotrichaceae; g_Turicibacter; | | | | |
| LKT_g_Turicibacter | | | | |
| k_Bacteria; p_Firmicutes; | 2.98 | Responder | 2.62 | 0.034 |
| c_Negativicutes; o_Selenomonadales; | | | | |
| f_Selenomonadaceae; g_Megamonas | | | | |
| k_Bacteria; p_Firmicutes; | 2.182 | Responder | 2.007 | 0.028 |
| c_Negativicutes; o_Selenomonadales; | | | | |
| f_Selenomonadaceae; g_Megamonas; | | | | |
| LKT_g_Megamonas | | | | |
| k_Bacteria; p_Firmicutes; | 2.487 | Responder | 2.171 | 0.004 |
| c_Negativicutes; o_Selenomonadales; | | | | |
| f_Selenomonadaceae; g_Megamonas; | | | | |
| LKT_s_Megamona_{—}rupellensis | | | | |
| k_Bacteria; p_Firmicutes; | 3.597 | Responder | 2.936 | 0.049 |
| c_Negativicutes; o_Veillonellales | | | | |
| k_Bacteria; p_Firmicutes; | 3.597 | Responder | 2.936 | 0.049 |
| c_Negativicutes; o_Veillonellales; | | | | |
| f_Veillonellaceae | | | | |
| k_Bacteria; p_Firmicutes; | 1.305 | Responder | 2.145 | 0.008 |
| c_Tissierellia; o_Tissierellales; | | | | |
| f_Peptoniphilaceae; g_Anaerococcus; | | | | |
| LKT_g_Anaerococcus | | | | |
| k_Bacteria; p_Firmicutes; | 0.475 | Responder | 2.339 | 0.011 |
| c_Tissierellia; o_Tissierellales; | | | | |
| f_Peptoniphilaceae; g_Anaerococcus; | | | | |
| LKT_s_Anaerococcus_{—}vaginalis | | | | |
| k_Bacteria; p_Firmicutes; | 0.925 | Responder | 2.274 | 0.011 |
| c_Tissierellia; o_Tissierellales; | | | | |
| f_Peptoniphilaceae; g_Finegoldia | | | | |
| k_Bacteria; p_Firmicutes; | 0.925 | Responder | 2.274 | 0.011 |
| c_Tissierellia; o_Tissierellales; | | | | |
| f_Peptoniphilaceae; g_Finegoldia; | | | | |
| LKT_s_Finegoldia_{—}magna | | | | |
| k_Bacteria; p_Fusobacteria; | 1.73 | Responder | 2.181 | 0.001 |
| c_Fusobacteriia; | | | | |
| o_Fusobacteriales; f_Fusobacteriaceae; | | | | |
| g_Fusobacterium; | | | | |
| LKT_s_Fusobacterium_{—}varium | | | | |
| k_Bacteria; p_Proteobacteria; | 0.141 | Responder | 3.018 | 0.002 |
| c_Betaproteobacteria; o_Burkholderiales; | | | | |
| f_Comamonadaceae; g_Variovorax; | | | | |
| LKT_g_Variovorax | | | | |
| k_Bacteria; p_Proteobacteria; | 0.852 | Responder | 2.383 | 0.048 |
| c_Betaproteobacteria; o_Burkholderiales; | | | | |
| f_Oxalobacteraceae; g_Janthinobacterium; | | | | |
| LKT_s_Janthinobacterium_Unclassified | | | | |

LDAscores computed for differentially abundant taxa calculated on batch-corrected metagenomic data obtained from five combined melanoma patient cohorts. Taxa with p ≤= 0.05 for the Kruskal-Wallis H statistic and LDA score >2 are listed.

TABLE 8
Taxa associated with improved PFS in the Houston cohort

| | | Biomarker < | | Biomarker >= | | | | P- | |
|---|---|---|---|---|---|---|---|---|---|
| LKT | Cutpoint | cutpoint | | cutpoint | | HR | CI | value | FDR |
| LKT_s_Pseudomonas_aeruginosa | 110 | 11 | (46%) | 13 | (54%) | 2.18E-10 | (0-Inf) | 0 | 0.003 |
| LKT_s_Massilistercora_timonensis | 18 | 9 | (38%) | 15 | (62%) | 0.0422 | (0.0052-0.343) | 0 | 0.004 |
| LKT_d_Eukaryota | 66 | 12 | (50%) | 12 | (50%) | 3.65E-10 | (0-Inf) | 0 | 0.004 |
| LKT_s_Eisenbergiella_massiliensis | 19 | 7 | (29%) | 17 | (71%) | 0.119 | (0.0323-0.44) | 0 | 0.01 |
| LKT_g_Eubacterium | 13 | 10 | (42%) | 14 | (58%) | 0.0997 | (0.0211-0.47) | 0 | 0.015 |
| LKT_s_Hungatella_hathewayi | 286 | 7 | (29%) | 17 | (71%) | 0.132 | (0.0359-0.486) | 0 | 0.015 |
| LKT_s_Anaerotignum_lactatifermentans | 49 | 7 | (29%) | 17 | (71%) | 0.123 | (0.0306-0.492) | 0.001 | 0.017 |
| LKT_s_Butyrivibrio_crossotus | 6 | 10 | (42%) | 14 | (58%) | 0.106 | (0.0221-0.506) | 0.001 | 0.021 |
| LKT_s_Intestinimonas_timonensis | 232 | 10 | (42%) | 14 | (58%) | 0.109 | (0.0228-0.523) | 0.001 | 0.025 |
| LKT_s_Lactobacillus_rhamnosus | 1 | 4 | (17%) | 20 | (83%) | 0.144 | (0.0371-0.559) | 0.001 | 0.031 |
| LKT_s_Parabacteroides_Unclassified | 160 | 4 | (17%) | 20 | (83%) | 0.15 | (0.0393-0.574) | 0.001 | 0.033 |
| LKT_s_Clostridium_spiroforme | 26 | 11 | (46%) | 13 | (54%) | 0.12 | (0.0252-0.569) | 0.002 | 0.036 |
| LKT_s_Bariatricus_massiliensis | 2 | 6 | (25%) | 18 | (75%) | 0.139 | (0.0328-0.588) | 0.002 | 0.036 |
| LKT_s_Erysipelotrichaceae_bacterium | 51 | 8 | (33%) | 16 | (67%) | 0.157 | (0.0422-0.586) | 0.002 | 0.036 |
| LKT_s_Blautia_schinkii | 19 | 11 | (46%) | 13 | (54%) | 0.122 | (0.0253-0.589) | 0.002 | 0.036 |
| LKT_s_Holdemanella_biformis | 5 | 13 | (54%) | 11 | (46%) | 0.0784 | (0.0099-0.622) | 0.002 | 0.036 |
| LKT_s_Butyrivibrio_Unclassified | 109 | 16 | (67%) | 8 | (33%) | 5.96E-10 | (0-Inf) | 0.002 | 0.036 |
| LKT_s_Intestinibacillus_massiliensis | 6 | 11 | (46%) | 13 | (54%) | 0.115 | (0.0227-0.585) | 0.002 | 0.036 |
| LKT_s_Fournierella_massiliensis | 105 | 15 | (62%) | 9 | (38%) | 7.18E-10 | (0-Inf) | 0.003 | 0.036 |
| LKT_g_Pseudoflavonifractor | 21 | 9 | (38%) | 15 | (62%) | 0.149 | (0.0367-0.607) | 0.003 | 0.036 |
| LKT_s_Ruminococcus_flavefaciens | 41 | 6 | (25%) | 18 | (75%) | 0.176 | (0.0492-0.629) | 0.003 | 0.036 |
| LKT_s_Blautia_Unclassified | 945 | 11 | (46%) | 13 | (54%) | 0.133 | (0.0282-0.626) | 0.003 | 0.036 |
| LKT_s_Subdoligranulum_Unclassified | 138 | 6 | (25%) | 18 | (75%) | 0.166 | (0.0433-0.635) | 0.003 | 0.036 |
| LKT_s_Blautia_hydrogenotrophica | 7 | 6 | (25%) | 18 | (75%) | 0.149 | (0.0352-0.634) | 0.003 | 0.036 |
| LKT_s_Eubacterium_ruminantium | 4 | 16 | (67%) | 8 | (33%) | 6.56E-10 | (0-Inf) | 0.003 | 0.036 |
| LKT_s_Pseudoflavonifractor_Unclassified | 95 | 8 | (33%) | 16 | (67%) | 0.159 | (0.0394-0.642) | 0.004 | 0.038 |
| LKT_s_Clostridioides_difficile | 172 | 8 | (33%) | 16 | (67%) | 0.188 | (0.0536-0.657) | 0.004 | 0.039 |
| LKT_g_Blautia | 769 | 9 | (38%) | 15 | (62%) | 0.167 | (0.0428-0.65) | 0.004 | 0.039 |
| LKT_g_Clostridium | 951 | 9 | (38%) | 15 | (62%) | 0.171 | (0.0437-0.67) | 0.004 | 0.041 |
| LKT_s_Streptomyces_Unclassified | 24 | 13 | (54%) | 11 | (46%) | 0.0894 | (0.0112-0.716) | 0.005 | 0.044 |
| LKT_f_Erysipelotrichaceae | 86 | 6 | (25%) | 18 | (75%) | 0.194 | (0.0546-0.692) | 0.005 | 0.047 |
| LKT_s_Eubacteriaceae_bacterium | 244 | 13 | (54%) | 11 | (46%) | 0.0913 | (0.0114-0.731) | 0.005 | 0.047 |
| LKT_s_Angelakisella_massiliensis | 239 | 16 | (67%) | 8 | (33%) | 8.21E-10 | (0-Inf) | 0.006 | 0.048 |
| LKT_s_Gordonibacter_urolithinfaciens | 5 | 8 | (33%) | 16 | (67%) | 0.196 | (0.0549-0.698) | 0.006 | 0.048 |
| LKT_s_Frisingicoccus_caecimuris | 5 | 6 | (25%) | 18 | (75%) | 0.199 | (0.0557-0.711) | 0.006 | 0.051 |
| LKT_s_Clostridium_scindens | 25 | 6 | (25%) | 18 | (75%) | 0.202 | (0.0565-0.718) | 0.007 | 0.051 |
| LKT_s_Bacillus_Unclassified | 13 | 12 | (50%) | 12 | (50%) | 0.153 | (0.0327-0.719) | 0.007 | 0.051 |
| LKT_s_Harryflintia_acetispora | 141 | 16 | (67%) | 8 | (33%) | 7.59E-10 | (0-Inf) | 0.007 | 0.053 |
| LKT_s_Bifidobacterium_pseudocatenulatum | 3 | 15 | (62%) | 9 | (38%) | 0.101 | (0.0128-0.794) | 0.008 | 0.056 |
| LKT_s_Faecalibacterium_Unclassified | 282 | 10 | (42%) | 14 | (58%) | 0.189 | (0.0479-0.744) | 0.008 | 0.059 |
| LKT_s_Eubacterium_Unclassified | 26 | 10 | (42%) | 14 | (58%) | 0.182 | (0.0446-0.742) | 0.008 | 0.059 |
| LKT_s_Ruminococcus_Unclassified | 3126 | 11 | (46%) | 13 | (54%) | 0.162 | (0.034-0.767) | 0.009 | 0.062 |
| LKT_g_Coprobacillus | 82 | 15 | (62%) | 9 | (38%) | 0.105 | (0.0133-0.826) | 0.009 | 0.062 |
| LKT_s_Gemmiger_formicilis | 1832 | 15 | (62%) | 9 | (38%) | 0.108 | (0.0137-0.844) | 0.01 | 0.063 |
| LKT_s_Sharpea_azabuensis | 10 | 16 | (67%) | 8 | (33%) | 8.49E-10 | (0-Inf) | 0.01 | 0.063 |
| LKT_s_Clostridium_botulinum | 33 | 16 | (67%) | 8 | (33%) | 0.103 | (0.0128-0.825) | 0.01 | 0.063 |
| LKT_s_Clostridium_symbiosum | 126 | 5 | (21%) | 19 | (79%) | 0.212 | (0.0582-0.773) | 0.01 | 0.063 |
| LKT_o_Actinobacteria | 93 | 17 | (71%) | 7 | (29%) | 8.88E-10 | (0-Inf) | 0.01 | 0.063 |
| LKT_f_Eubacteriaceae | 60 | 10 | (42%) | 14 | (58%) | 0.198 | (0.0505-0.779) | 0.011 | 0.064 |
| LKT_s_Parabacteroides_distasonis | 1643 | 4 | (17%) | 20 | (83%) | 0.222 | (0.0621-0.79) | 0.011 | 0.065 |
| LKT_s_Coprobacillus_Unclassified | 12 | 12 | (50%) | 12 | (50%) | 0.17 | (0.0364-0.798) | 0.011 | 0.066 |
| LKT_s_Ruminococcus_bromii | 376 | 17 | (71%) | 7 | (29%) | 9.58E-10 | (0-Inf) | 0.014 | 0.075 |
| LKT_g_Bacillus | 34 | 14 | (58%) | 10 | (42%) | 0.117 | (0.0149-0.914) | 0.014 | 0.075 |
| LKT_s_Anaerotruncus_massiliensis | 7 | 10 | (42%) | 14 | (58%) | 0.209 | (0.053-0.827) | 0.015 | 0.075 |
| LKT_s_Clostridium_Unclassified | 8337 | 14 | (58%) | 10 | (42%) | 0.116 | (0.0146-0.922) | 0.015 | 0.075 |
| LKT_s_Muribaculaceae_bacterium | 8 | 12 | (50%) | 12 | (50%) | 0.179 | (0.0383-0.841) | 0.015 | 0.075 |
| LKT_s_Clostridium_sporosphaeroides | 20 | 14 | (58%) | 10 | (42%) | 0.116 | (0.0146-0.922) | 0.015 | 0.075 |
| LKT_s_Lachnoclostridium_phocaeense | 27 | 14 | (58%) | 10 | (42%) | 0.116 | (0.0146-0.922) | 0.015 | 0.075 |
| LKT_s_Butyricicoccus_Unclassified | 762 | 15 | (62%) | 9 | (38%) | 0.117 | (0.0147-0.934) | 0.016 | 0.078 |
| LKT_s_Christensenella_minuta | 5 | 15 | (62%) | 9 | (38%) | 0.117 | (0.0147-0.934) | 0.016 | 0.078 |
| LKT_s_Anaerofustis_stercorihominis | 31 | 15 | (62%) | 9 | (38%) | 0.12 | (0.0153-0.941) | 0.016 | 0.079 |
| LKT_s_Phascolarctobacterium_succinatutens | 6 | 11 | (46%) | 13 | (54%) | 0.226 | (0.0593-0.86) | 0.018 | 0.085 |
| LKT_s_Blautia_producta | 21 | 8 | (33%) | 16 | (67%) | 0.239 | (0.0665-0.863) | 0.018 | 0.086 |
| LKT_s_Ruminococcus_albus | 11 | 11 | (46%) | 13 | (54%) | 0.22 | (0.0553-0.872) | 0.019 | 0.089 |
| LKT_o_Bacillales | 19 | 15 | (62%) | 9 | (38%) | 0.125 | (0.016-0.986) | 0.019 | 0.089 |
| LKT_s_Eubacterium_limosum | 3 | 13 | (54%) | 11 | (46%) | 0.185 | (0.0386-0.892) | 0.02 | 0.089 |
| LKT_s_Clostridium_methylpentosum | 23 | 13 | (54%) | 11 | (46%) | 0.18 | (0.0366-0.887) | 0.02 | 0.089 |
| LKT_s_Clostridium_leptum | 444 | 15 | (62%) | 9 | (38%) | 0.123 | (0.0153-0.985) | 0.02 | 0.089 |
| LKT_s_Intestinimonas_butyriciproducens | 57 | 6 | (25%) | 18 | (75%) | 0.23 | (0.0597-0.886) | 0.02 | 0.09 |
| LKT_s_Coprobacter_fastidiosus | 12 | 11 | (46%) | 13 | (54%) | 0.226 | (0.0569-0.898) | 0.022 | 0.094 |
| LKT_s_Intestinimonas_massiliensis | 226 | 15 | (62%) | 9 | (38%) | 0.13 | (0.0165-1.02) | 0.022 | 0.094 |
| LKT_s_Anaerotruncus_colihominis | 396 | 9 | (38%) | 15 | (62%) | 0.248 | (0.0686-0.897) | 0.022 | 0.094 |
| LKT_g_Parabacteroides | 2274 | 11 | (46%) | 13 | (54%) | 0.238 | (0.0625-0.905) | 0.022 | 0.094 |
| LKT_s_Lachnospira_mutipara | 13 | 19 | (79%) | 5 | (21%) | 1.03E-09 | (0-Inf) | 0.023 | 0.096 |
| LKT_s_Coprococcus_catus | 236 | 19 | (79%) | 5 | (21%) | 1.03E-09 | (0-Inf) | 0.023 | 0.096 |
| LKT_f_Ruminococcaceae | 2425 | 12 | (50%) | 12 | (50%) | 0.235 | (0.0601-0.919) | 0.025 | 0.1 |
| LKT_g_Faecalibacterium | 1302 | 17 | (71%) | 7 | (29%) | 0.132 | (0.0165-1.05) | 0.026 | 0.1 |
| LKT_s_Agathobaculum_desmolans | 83 | 18 | (75%) | 6 | (25%) | 2.95E-09 | (0-Inf) | 0.028 | 0.109 |
| LKT_s_Sporobacter_termitidis | 57 | 19 | (79%) | 5 | (21%) | 2.96E-09 | (0-Inf) | 0.03 | 0.113 |
| LKT_s_Coprococcus_Unclassified | 33 | 8 | (33%) | 16 | (67%) | 0.261 | (0.0709-0.963) | 0.031 | 0.117 |
| LKT_s_Anaeromassilibacillus_senegalensis | 17 | 14 | (58%) | 10 | (42%) | 0.212 | (0.0447-1) | 0.032 | 0.12 |
| LKT_s_Bacteroidales_bacterium | 6 | 12 | (50%) | 12 | (50%) | 0.244 | (0.0612-0.977) | 0.032 | 0.12 |
| LKT_g_Butyricicoccus | 179 | 17 | (71%) | 7 | (29%) | 0.145 | (0.0184-1.14) | 0.034 | 0.124 |
| LKT_s_Neglecta_timonensis | 117 | 15 | (62%) | 9 | (38%) | 0.143 | (0.0178-1.15) | 0.035 | 0.124 |
| LKT_s_Bifidobacterium_longum | 253 | 17 | (71%) | 7 | (29%) | 0.146 | (0.0185-1.15) | 0.035 | 0.124 |
| LKT_s_Ruthenibacterium_lactatiformans | 137 | 12 | (50%) | 12 | (50%) | 0.262 | (0.0684-1) | 0.036 | 0.126 |
| LKT_s_Clostridium_phoceensis | 277 | 6 | (25%) | 18 | (75%) | 0.281 | (0.0792-0.995) | 0.036 | 0.126 |
| LKT_s_Cuneatibacter_caecimuris | 8 | 8 | (33%) | 16 | (67%) | 0.283 | (0.0803-0.995) | 0.037 | 0.126 |
| LKT_s_Clostridium_innocuum | 8 | 7 | (29%) | 17 | (71%) | 0.282 | (0.0797-0.997) | 0.037 | 0.126 |
| LKT_s_Phascolarctobacterium_faecium | 917 | 14 | (58%) | 10 | (42%) | 0.224 | (0.0483-1.04) | 0.037 | 0.126 |
| LKT_s_Dorea_Unclassified | 95 | 13 | (54%) | 11 | (46%) | 0.221 | (0.0466-1.05) | 0.038 | 0.127 |
| LKT_s_Alistipes_indistinctus | 69 | 16 | (67%) | 8 | (33%) | 0.147 | (0.0183-1.18) | 0.039 | 0.129 |
| LKT_s_Blautia_obeum | 1058 | 14 | (58%) | 10 | (42%) | 0.224 | (0.0471-1.06) | 0.04 | 0.131 |
| LKT_s_Holdemania_filiformis | 18 | 10 | (42%) | 14 | (58%) | 0.284 | (0.0789-1.03) | 0.041 | 0.134 |
| LKT_s_Clostridium_disporicum | 24 | 15 | (62%) | 9 | (38%) | 0.226 | (0.0474-1.07) | 0.043 | 0.135 |
| LKT_s_Flintibacter_Unclassified | 38 | 12 | (50%) | 12 | (50%) | 0.263 | (0.0661-1.04) | 0.043 | 0.135 |
| LKT_s_Butyricicoccus_pullicaecorum | 53 | 16 | (67%) | 8 | (33%) | 0.157 | (0.02-1.23) | 0.044 | 0.136 |
| LKT_p_Firmicutes | 5337 | 15 | (62%) | 9 | (38%) | 0.231 | (0.0493-1.08) | 0.044 | 0.136 |
| LKT_s_Bifidobacterium_bifidum | 2 | 14 | (58%) | 10 | (42%) | 0.233 | (0.0496-1.09) | 0.045 | 0.137 |
| LKT_s_Bifidobacterium_adolescentis | 592 | 20 | (83%) | 4 | (17%) | 3.11E-09 | (0-Inf) | 0.046 | 0.139 |
| LKT_s_Paenibacillus_Unclassified | 17 | 15 | (62%) | 9 | (38%) | 0.235 | (0.0503-1.1) | 0.046 | 0.139 |
| LKT_s_Oscillibacter_ruminantium | 554 | 17 | (71%) | 7 | (29%) | 0.16 | (0.0204-1.26) | 0.047 | 0.139 |
| LKT_s_Oscillibacter_valericigenes | 368 | 17 | (71%) | 7 | (29%) | 0.16 | (0.0204-1.26) | 0.047 | 0.139 |
| LKT_s_Ruminococcaceae_bacterium | 5466 | 16 | (67%) | 8 | (33%) | 0.162 | (0.0206-1.27) | 0.048 | 0.139 |
| LKT_s_Firmicutes_bacterium | 242 | 8 | (33%) | 16 | (67%) | 0.314 | (0.0935-1.05) | 0.049 | 0.139 |
| LKT_s_Coprococcus_eutactus_Unclassified | 70 | 12 | (50%) | 12 | (50%) | 0.282 | (0.0739-1.08) | 0.049 | 0.139 |
| LKT_Unclassified | 31450 | 7 | (29%) | 17 | (71%) | 0.301 | (0.085-1.07) | 0.05 | 0.139 |
| LKT_s_Phocea_massiliensis | 91 | 10 | (42%) | 14 | (58%) | 0.278 | (0.0711-1.09) | 0.05 | 0.139 |
| LKT_s_Alistipes_onderdonkii | 874 | 13 | (54%) | 11 | (46%) | 0.238 | (0.0502-1.13) | 0.05 | 0.139 |
| LKT_f_Clostridiaceae | 2055 | 14 | (58%) | 10 | (42%) | 0.241 | (0.0516-1.13) | 0.05 | 0.139 |
| LKT_s_Lactobacillus_paracasei | 1 | 16 | (67%) | 8 | (33%) | 0.164 | (0.021-1.29) | 0.05 | 0.139 |
| LKT_s_Lactococcus_lactis | 21 | 17 | (71%) | 7 | (29%) | 0.164 | (0.0209-1.29) | 0.051 | 0.139 |
| LKT_s_Mogibacterium_pumilum | 1 | 19 | (79%) | 5 | (21%) | 3.26E-09 | (0-Inf) | 0.051 | 0.139 |
| LKT_s_Barnesiella_intestinihominis | 5977 | 20 | (83%) | 4 | (17%) | 3.19E-09 | (0-Inf) | 0.051 | 0.139 |
| LKT_s_Bacteroides_xylanisolvens | 4939 | 13 | (54%) | 11 | (46%) | 0.241 | (0.0508-1.14) | 0.052 | 0.139 |
| LKT_s_Blautia_wexlerae | 350 | 9 | (38%) | 15 | (62%) | 0.306 | (0.0856-1.09) | 0.054 | 0.142 |
| LKT_s_Lactobacillus_vaginalis | 2 | 20 | (83%) | 4 | (17%) | 3.25E-09 | (0-Inf) | 0.056 | 0.142 |
| LKT_s_Clostridium_viride | 23 | 20 | (83%) | 4 | (17%) | 3.25E-09 | (0-Inf) | 0.056 | 0.142 |
| LKT_s_Agathobaculum_butyriciproducens | 144 | 18 | (75%) | 6 | (25%) | 0.167 | (0.021-1.33) | 0.056 | 0.142 |
| LKT_s_Faecalibacterium_prausnitzii | 24560 | 14 | (58%) | 10 | (42%) | 0.251 | (0.0542-1.16) | 0.056 | 0.142 |
| LKT_s_Anaerostipes_hadrus | 51 | 8 | (33%) | 16 | (67%) | 0.315 | (0.0898-1.1) | 0.057 | 0.143 |
| LKT_s_Marvinbryantia_formatexigens | 87 | 17 | (71%) | 7 | (29%) | 0.17 | (0.0213-1.35) | 0.058 | 0.145 |
| LKT_s_Ruminococcus_champanellensis | 24 | 14 | (58%) | 10 | (42%) | 0.256 | (0.0551-1.19) | 0.061 | 0.147 |
| LKT_s_Bacteroides_uniformis | 16740 | 6 | (25%) | 18 | (75%) | 0.3 | (0.0786-1.14) | 0.062 | 0.147 |
| LKT_s_Alistipes_senegalensis | 84 | 13 | (54%) | 11 | (46%) | 0.304 | (0.0801-1.15) | 0.063 | 0.147 |
| LKT_s_Clostridium_perfringens | 14 | 16 | (67%) | 8 | (33%) | 0.175 | (0.0219-1.39) | 0.063 | 0.147 |
| LKT_f_Oscillospiraceae | 584 | 7 | (29%) | 17 | (71%) | 0.322 | (0.0918-1.13) | 0.064 | 0.147 |
| LKT_s_Streptococcus_thermophilus | 42 | 18 | (75%) | 6 | (25%) | 0.176 | (0.0223-1.39) | 0.064 | 0.147 |
| LKT_f_Coriobacteriaceae | 638 | 18 | (75%) | 6 | (25%) | 0.176 | (0.0223-1.39) | 0.064 | 0.147 |
| LKT_s_Terrisporobacter_glycolicus | 1 | 13 | (54%) | 11 | (46%) | 0.256 | (0.0539-1.21) | 0.065 | 0.148 |
| LKT_s_Fusicatenibacter_saccharivorans | 356 | 13 | (54%) | 11 | (46%) | 0.259 | (0.0547-1.22) | 0.066 | 0.15 |

TABLE 9
Taxa associated with decreased PFS in the Houston cohort

| | | Biomarker < | | Biomarker >= | | | P- | |
|---|---|---|---|---|---|---|---|---|
| LKT | Cutpoint | Cutpoint | | Cutpoint | | CI | value | FDR |
| LKT_g_Oscillibacter | 1587 | 18 | (75%) | 6 | (25%) | (2.65-39.8) | 0 | 0.004 |
| LKT_g_Prevotella | 447 | 19 | (79%) | 5 | (21%) | (2.47-46.4) | 0 | 0.008 |
| LKT_s_Butyricimonas_virosa | 548 | 18 | (75%) | 6 | (25%) | (2.39-45.5) | 0 | 0.009 |
| LKT_s_Bacteroides_massiliensis | 2933 | 17 | (71%) | 7 | (29%) | (2.1-34.7) | 0 | 0.015 |
| LKT_s_Prevotella_lascolaii | 164 | 20 | (83%) | 4 | (17%) | (1.82-30.3) | 0.001 | 0.029 |
| LKT_s_Prevotella_stercorea | 104 | 19 | (79%) | 5 | (21%) | (1.59-19.9) | 0.003 | 0.036 |
| LKT_s_Bacteroides_plebeius | 530 | 9 | (38%) | 15 | (62%) | (0-Inf) | 0.004 | 0.038 |
| LKT_s_Prevotella_Unclassified | 147 | 15 | (62%) | 9 | (38%) | (1.54-22.8) | 0.004 | 0.038 |
| LKT_s_Prevotella_copri | 75 | 14 | (58%) | 10 | (42%) | (1.45-23) | 0.005 | 0.047 |
| LKT_s_Megasphaera_Unclassified | 1 | 19 | (79%) | 5 | (21%) | (1.33-19.3) | 0.008 | 0.059 |
| LKT_s_Parabacteroides_merdae | 11870 | 18 | (75%) | 6 | (25%) | (1.23-15.1) | 0.013 | 0.074 |
| LKT_g_Escherichia | 9 | 12 | (50%) | 12 | (50%) | (1.21-26.2) | 0.013 | 0.074 |
| LKT_s_Escherichia_coli | 47 | 11 | (46%) | 13 | (54%) | (1.06-22.9) | 0.024 | 0.097 |
| LKT_s_Streptococcus_parasanguinis | 24 | 17 | (71%) | 7 | (29%) | (1.09-12.4) | 0.026 | 0.1 |
| LKT_g_Coprococcus | 179 | 20 | (83%) | 4 | (17%) | (1.07-16.7) | 0.026 | 0.1 |
| LKT_s_Dialister_invisus | 2 | 8 | (33%) | 16 | (67%) | (0.838-51.5) | 0.039 | 0.129 |
| LKT_s_Desulfovibrio_fairfieldensis | 72 | 18 | (75%) | 6 | (25%) | (0.974-12.5) | 0.041 | 0.134 |
| LKT_s_Eubacterium_maltosivorans | 3 | 18 | (75%) | 6 | (25%) | (0.93-12.4) | 0.05 | 0.139 |
| LKT_s_Veillonella_parvula | 15 | 19 | (79%) | 5 | (21%) | (0.913-11.7) | 0.054 | 0.142 |
| LKT_g_Klebsiella | 141 | 19 | (79%) | 5 | (21%) | (0.913-11.7) | 0.054 | 0.142 |
| LKT_s_Acidaminococcus_intestini | 226 | 20 | (83%) | 4 | (17%) | (0.891-13.7) | 0.056 | 0.142 |
| LKT_s_Desulfovibrio_Unclassified | 22 | 16 | (67%) | 8 | (33%) | (0.899-10.9) | 0.059 | 0.147 |
| LKT_g_Dorea | 32 | 20 | (83%) | 4 | (17%) | (0.872-14.1) | 0.061 | 0.147 |
| LKT_s_Adlercreutzia_Unclassified | 8 | 12 | (50%) | 12 | (50%) | (0.877-12.6) | 0.061 | 0.147 |
| LKT_s_Asaccharobacter_celatus | 11 | 12 | (50%) | 12 | (50%) | (0.877-12.6) | 0.061 | 0.147 |
| LKT_s_Paraprevotella_clara | 309 | 18 | (75%) | 6 | (25%) | (0.896-10.2) | 0.063 | 0.147 |
| LKT_s_Candidatus_Stoquefichus | 14 | 20 | (83%) | 4 | (17%) | (0.854-13.4) | 0.066 | 0.15 |
| LKT_s_Prevotella_koreensis | 36 | 20 | (83%) | 4 | (17%) | (0.853-13.2) | 0.066 | 0.15 |

TABLE 10
Differentially abundant taxa between different clusters from AGP dataset identified using PhenoGraph R package

| | | LDA | |
|---|---|---|---|
| | | score | |
| Taxon | Microbiotype_classification | (log10) | p-value |
| k_Bacteria.p_Bacteroidetes.c_Bacteroidia.o_Bacteroidales. | Detrimental_1 | 3.184865 | 4.37E−19 |
| f_Bacteroidaceae.g_Bacteroides.s_Bacteroides_{—}intestinalis | | | |
| k_Bacteria.p_Bacteroidetes.c_Bacteroidia.o_Bacteroidales. | Detrimental_1 | 3.078344 | 3.92E−62 |
| f_Porphyromonadaceae.g_Barnesiella | | | |
| k_Bacteria.p_Firmicutes.c_Clostridia.o_Clostridiales. | Detrimental_1 | 3.665278 | 6.88E−107 |
| f_Ruminococcaceae.g_Ruminiclostridium. | | | |
| s_Eubacterium_{—}siraeum_V10Sc8a | | | |
| k_Bacteria.p_Firmicutes.c_Clostridia.o_Clostridiales. | Detrimental_1 | 3.6284 | 1.42E−48 |
| f_Ruminococcaceae.g_Ruminiclostridium | | | |
| k_Bacteria.p_Bacteroidetes.c_Bacteroidia.o_Bacteroidales. | Detrimental_1 | 3.327553 | 1.92E−62 |
| f_Rikenellaceae.g_Alistipes.s_Alistipes_{—}putredinis | | | |
| k_Bacteria.p_Bacteroidetes.c_Bacteroidia.o_Bacteroidales. | Detrimental_1 | 3.308983 | 8.92E−22 |
| f_Porphyromonadaceae.g_Parabacteroides. | | | |
| s_Parabacteroides_{—}goldsteinii | | | |
| k_Bacteria.p_Bacteroidetes.c_Bacteroidia.o_Bacteroidales. | Detrimental_1 | 3.860989 | 1.28E−57 |
| f_Bacteroidaceae.g_Bacteroides.s_Bacteroides_{—}stercoris | | | |
| k_Bacteria.p_Bacteroidetes.c_Bacteroidia.o_Bacteroidales. | Detrimental_1 | 4.006943 | 4.97E−43 |
| f_Bacteroidaceae.g_Bacteroides.s_Bacteroides_{—}massiliensis | | | |
| k_Bacteria.p_Bacteroidetes.c_Bacteroidia.o_Bacteroidales. | Detrimental_1 | 3.987259 | 4.54E−60 |
| f_Bacteroidaceae.g_Bacteroides.s_Bacteroides_{—}uniformis | | | |
| k_Bacteria.p_Bacteroidetes.c_Bacteroidia.o_Bacteroidales. | Detrimental_1 | 3.912724 | 9.98E−49 |
| f_Rikenellaceae.g_Alistipes | | | |
| k_Bacteria.p_Bacteroidetes.c_Bacteroidia.o_Bacteroidales. | Detrimental_1 | 3.912724 | 9.98E−49 |
| f_Rikenellaceae | | | |
| k_Bacteria.p_Bacteroidetes.c_Bacteroidia.c_Bacteroidales. | Detrimental_2 | 3.046337 | 2.61E−06 |
| f_Prevotellaceae.g_Prevotella.s_Prevotella_{—}stercorea | | | |
| k_Bacteria.p_Firmicutes.c_Clostridia.o_Clostridiales. | Detrimental_2 | 3.229244 | 2.61E−72 |
| f_Lachnospiraceae.g_Butyrivibrio.s_Butyrivibrio_{—}crossotus | | | |
| k_Bacteria.p_Firmicutes.c_Clostridia.o_Clostridiales. | Detrimental_2 | 3.231967 | 2.83E−77 |
| f_Lachnospiraceae.g_Butyrivibrio | | | |
| k_Bacteria.p_Bacteroidetes.c_Bacteroidia.o_Bacteroidales. | Detrimental_2 | 3.409527 | 2.01E−46 |
| f_Bacteroidaceae.g_Bacteroides.s_Bacteroides_{—}coprophilus | | | |
| k_Bacteria.p_Bacteroidetes.c_Bacteroidia.o_Bacteroidales. | Detrimental_2 | 3.410771 | 4.05E−45 |
| f_Bacteroidaceae.g_Bacteroides.s_Bacteroides_{—}coprocola | | | |
| k_Bacteria.p_Firmicutes.c_Clostridia.o_Clostridiales. | Detrimental_2 | 3.793237 | 3.47E−35 |
| f_Lachnospiraceae.g_Roseburia.s_Roseburia_{—}inulinivorans | | | |
| k_Bacteria.p_Bacteroidetes.c_Bacteroidia.o_Bacteroidales. | Detrimental_2 | 3.395656 | 4.63E−88 |
| f_Prevotellaceae.g_Paraprevotella.s_Paraprevotella_{—}clara | | | |
| k_Bacteria.p_Bacteroidetes.c_Bacteroidia.o_Bacteroidales. | Detrimental_2 | 3.429307 | 1.68E−98 |
| f_Prevotellaceae.g_Paraprevotella | | | |
| k_Bacteria.p_Firmicutes.c_Negativicutes.o_Selenomonadales. | Detrimental_2 | 3.608582 | 2.90E−32 |
| f_Veillonellaceae | | | |
| k_Bacteria.p_Bacteroidetes.c_Bacteroidia.o_Bacteroidales. | Detrimental_2 | 4.154545 | 1.26E−165 |
| f_Bacteroidaceae.g_Bacteroides.s_Bacteroides_{—}plebeius | | | |
| k_Bacteria.p_Bacteroidetes.c_Bacteroidia.o_Bacteroidales. | Detrimental_2 | 4.804878 | 2.23E−265 |
| f_Prevotellaceae.g_Prevotella.s_Prevotella_{—}copri | | | |
| k_Bacteria.p_Bacteroidetes.c_Bacteroidia.o_Bacteroidales. | Detrimental_2 | 4.858163 | 3.10E−259 |
| f_Prevotellaceae.g_Prevotella | | | |
| k_Bacteria.p_Bacteroidetes.c_Bacteroidia.o_Bacteroidales. | Detrimental_2 | 4.873942 | 2.91E−298 |
| f_Prevotellaceae | | | |
| k_Bacteria.p_Bacteroidetes.c_Bacteroidia.o_Bacteroidales | Detrimental_2 | 4.579688 | 1.08E−20 |
| k_Bacteria.p_Bacteroidetes.c_Bacteroidia | Detrimental_2 | 4.579688 | 1.08E−20 |
| k_Bacteria.p_Bacteroidetes | Detrimental_2 | 4.570155 | 7.96E−20 |
| k_Bacteria.p_Firmicutes.c_Clostridia.o_Clostridiales. | Beneficial_1 | 3.797825 | 7.46E−49 |
| f_Lachnospiraceae.g_Roseburia.s_Roseburia_{—}intestinalis | | | |
| k_Bacteria.p_Firmicutes.c_Clostridia.o_Clostridiales. | Beneficial_1 | 3.515375 | 2.34E−52 |
| f_Ruminococcaceae.g_Ruminococcus.s_Ruminococcus_{—}bromii | | | |
| k_Bacteria.p_Firmicutes.c_Clostridia.o_Clostridiales. | Beneficial_1 | 3.708043 | 3.16E−177 |
| f_Lachnospiraceae.g_Coprococcus.s_Coprococcus_{—}eutactus | | | |
| k_Bacteria.p_Firmicutes.c_Clostridia.o_Clostridiales. | Beneficial_1 | 3.704216 | 1.09E−132 |
| f_Lachnospiraceae.g_Coprococcus | | | |
| k_Bacteria.p_Bacteroidetes.c_Bacteroidia.o_Bacteroidales. | Beneficial_1 | 3.852179 | 1.83E−54 |
| f_Bacteroidaceae.g_Bacteroides.s_Bacteroides_{—}eggerthii | | | |
| k_Bacteria.p_Firmicutes.c_Clostridia.o_Clostridiales. | Beneficial_1 | 3.801353 | 6.00E−39 |
| f_Lachnospiraceae.g_Roseburia | | | |
| k_Bacteria.p_Firmicutes.c_Clostridia.o_Clostridiales. | Beneficial_1 | 3.789677 | 1.16E−21 |
| f_Eubacteriaceae.g_Eubacterium.s_Eubacterium_{—}eligens | | | |
| k_Bacteria.p_Firmicutes.c_Clostridia.o_Clostridiales. | Beneficial_1 | 3.613305 | 1.66E−17 |
| f_Eubacteriaceae.g_Eubacterium | | | |
| k_Bacteria.p_Firmicutes.c_Clostridia.o_Clostridiales. | Beneficial_1 | 3.613305 | 1.66E−17 |
| f_Eubacteriaceae | | | |
| k_Bacteria.p_Firmicutes.c_Clostridia.o_Clostridiales. | Beneficial_1 | 3.66337 | 4.23E−101 |
| f_Ruminococcaceae.g_Ruminococcus | | | |
| k_Bacteria.p_Firmicutes.c_Clostridia.o_Clostridiales. | Beneficial_1 | 4.243499 | 2.09E−98 |
| f_Ruminococcaceae.g_Faecalibacterium.s_Faecalibacterium_{—}prausnitzii | | | |
| k_Bacteria.p_Firmicutes.c_Clostridia.o_Clostridiales. | Beneficial_1 | 4.243499 | 2.09E−98 |
| f_Ruminococcaceae.g_Faecalibacterium | | | |
| k_Bacteria.p_Firmicutes.c_Clostridia.o_Clostridiales. | Beneficial_1 | 4.505908 | 1.73E−80 |
| f_Ruminococcaceae | | | |
| k_Bacteria.p_Firmicutes.c_Clostridia.o_Clostridiales | Beneficial_1 | 4.65113 | 2.51E−34 |
| k_Bacteria.p_Firmicutes.c_Clostridia | Beneficial_1 | 4.651188 | 2.53E−34 |
| k_Bacteria.p_Firmicutes | Beneficial_1 | 4.594075 | 2.06E−26 |
| k_Bacteria.p_Firmicutes.c_Clostridia.o_Clostridiales. | Beneficial_2 | 3.488811 | 4.94E−44 |
| f_Ruminococcaceae.g_Ruminiclostridium.s_Clostridium_{—}leptum | | | |
| k_Bacteria.p_Firmicutes.c_Clostridia.o_Clostridiales. | Beneficial_2 | 3.748186 | 1.43E−14 |
| f_Eubacteriaceae.g_Eubacterium.s_Eubacterium_{—}coprostanoligenes | | | |
| k_Bacteria.p_Firmicutes.c_Erysipelotrichia.o_Erysipelotrichales. | Beneficial_2 | 3.083136 | 2.85E−173 |
| f_Erysipelotrichaceae.g_Erysipelatoclostridium. | | | |
| s_Clostridium_{—}innocuum | | | |
| k_Bacteria.p_Firmicutes.c_Negativicutes.o_Selenomonadales. | Beneficial_2 | 3.034274 | 1.59E−09 |
| f_Veillonellaceae.g_Veillonella | | | |
| k_Bacteria.p_Bacteroidetes.c_Bacteroidia.o_Bacteroidales. | Beneficial_2 | 3.095098 | 0.003244 |
| f_Porphyromonadaceae.g_Dysgonomonas.s_Dysgonomonas_{—}capnocytophagoides | | | |
| k_Bacteria.p_Firmicutes.c_Bacilli.o_Lactobacillies. | Beneficial_2 | 3.016357 | 2.35E−40 |
| f_Streptococcaceae.g_Streptococcus.s_Streptococcus_{—}salivarius | | | |
| k_Bacteria.p_Firmicutes.c_Bacilli.o_Lactobacillies. | Beneficial_2 | 3.048689 | 0.000318 |
| f_Lactobacillaceae | | | |
| k_Bacteria.p_Firmicutes.c_Bacilli.o_Bacillales.f_Bacillaceae | Beneficial_2 | 3.184483 | 7.92E−11 |
| k_Bacteria.p_Firmicutes.c_Clostridia.o_Clostridiales. | Beneficial_2 | 3.058639 | 6.80E−30 |
| f_Peptostreptococcaceae | | | |
| k_Bacteria.p_Bacteroidetes.c_Bacteroidia.o_Bacteroidales. | Beneficial_2 | 3.273405 | 0.0025 |
| f_Porphyromonadaceae.g_Dysgonomonas | | | |
| k_Bacteria.p_Firmicutes.c_Bacilli.o_Lactobacillies. | Beneficial_2 | 3.191879 | 5.09E−53 |
| f_Streptococcaceae.g_Streptococcus | | | |
| k_Bacteria.p_Bacteroidetes.c_Bacteroidia.o_Bacteroidales. | Beneficial_2 | 3.142528 | 1.45E−28 |
| f_Rikenellaceae.g_Alistipes.s_Alistipes_{—}finegoldii | | | |
| k_Bacteria.p_Firmicutes.c_Clostridia.o_Clostridiales. | Beneficial_2 | 3.225547 | 2.00E−52 |
| f_Clostridiaceae.g_Hungatella | | | |
| k_Bacteria.p_Firmicutes.c_Clostridia.o_Clostridiales. | Beneficial_2 | 3.225547 | 2.00E−52 |
| f_Clostridiaceae.g_Hungatella.s_Clostridium_{—}hathewayi | | | |
| k_Bacteria.p_Firmicutes.c_Bacilli.o_Lactobacillus. | Beneficial_2 | 3.274215 | 1.13E−56 |
| f_Streptococcaceae | | | |
| k_Bacteria.p_Firmicutes.c_Clostridia.o_Clostridiales. | Beneficial_2 | 3.055903 | 2.71E−14 |
| f_Lachnospiraceae.g_Eisenbergiella.s_Eisenbergiella_{—}tayi | | | |
| k_Bacteria.p_Firmicutes.c_Clostridia.o_Clostridiales. | Beneficial_2 | 3.055903 | 2.71E−14 |
| f_Lachnospiraceae.g_Eisenbergiella | | | |
| k_Bacteria.p_Firmicutes.c_Clostridia.o_Clostridiales. | Beneficial_2 | 3.357975 | 9.80e−311 |
| f_Lachnospiraceae.g_Lachnospiraceae.s_Clostridium_{—}bolteae | | | |
| k_Bacteria.p_Firmicutes.c_Clostridia.o_Clostridiales. | Beneficial_2 | 3.382746 | 0 |
| f_Flavonifractor.g_Flavonifractor | | | |
| k_Bacteria.p_Firmicutes.c_Clostridia.o_Clostridiales. | Beneficial_2 | 3.382746 | 0 |
| f_Flavonifractor | | | |
| k_Bacteria.p_Firmicutes.c_Clostridia.o_Clostridiales. | Beneficial_2 | 3.382746 | 0 |
| f_Flavonifractor.g_Flavonifractor.s_Flavonifractor_{—}plautii | | | |
| k_Bacteria.p_Firmicutes.c_Clostridia.o_Clostridiales. | Beneficial_2 | 3.392874 | 2.99E−158 |
| f_Ruminococcaceae.g_Subdoligranulum.s_Subdoligranulum_{—}variabile | | | |
| k_Bacteria.p_Firmicutes.c_Clostridia.o_Clostridiales. | Beneficial_2 | 3.392874 | 2.99E−158 |
| f_Ruminococcaceae.g_Subdoligranulum | | | |
| k_Bacteria.p_Firmicutes.c_Clostridia.o_Clostridiales. | Beneficial_2 | 3.17349 | 5.57E−23 |
| f_Lachnospiraceae.g_Blautia.s_Blautia_{—}faecis | | | |
| k_Bacteria.p_Firmicutes.c_Erysipelotrichia.o_Erysipelotrichales. | Beneficial_2 | 3.508658 | 0 |
| f_Erysipelotrichaceae.g_Erysipelatoclostridium | | | |
| k_Bacteria.p_Firmicutes.c_Clostridia.o_Clostridiales. | Beneficial_2 | 3.422413 | 6.63E−214 |
| f_Lachnospiraceae.g_Lachnospiraceae | | | |
| k_Bacteria.p_Firmicutes.c_Negativicutes.o_Selenomonadales. | Beneficial_2 | 3.321573 | 5.68E−21 |
| f_Veillonellaceae.g_Dialister.s_Dialister_{—}invisus | | | |
| k_Bacteria.p_Firmicutes.c_Clostridia.o_Clostridiales. | Beneficial_2 | 3.419739 | 3.42E−91 |
| f_Lachnospiraceae.g_Anaerostipes.s_Anaerostipes_{—}caccae | | | |
| k_Bacteria.p_Firmicutes.c_Clostridia.o_Clostridiales. | Beneficial_2 | 3.419739 | 3.42E−91 |
| f_Lachnospiraceae.g_Anaerostipes | | | |
| k_Bacteria.p_Firmicutes.c_Bacilli.o_Bacillales | Beneficial_2 | 3.600549 | 2.92E−101 |
| k_Bacteria.p_Firmicutes.c_Clostridia.o_Clostridiales. | Beneficial_2 | 3.594564 | 3.96E−139 |
| f_Lachnospiraceae.g_Blautia.s_Ruminococcus_{—}torques | | | |
| k_Bacteria.p_Firmicutes.c_Clostridia.o_Clostridiales. | Beneficial_2 | 3.390281 | 2.50E−26 |
| f_Clostridiaceae | | | |
| k_Bacteria.p_Bacteroidetes.c_Bacteroidia.o_Bacteroidales. | Beneficial_2 | 3.444473 | 9.19E−21 |
| f_Bacteroidaceae.g_Bacteroides.s_Bacteroides_{—}cellulosilyticus | | | |
| k_Bacteria.p_Bacteroidetes.c_Bacteroidia.o_Bacteroidales. | Beneficial_2 | 3.532307 | 3.59E−24 |
| f_Rikenellaceae.g_Alistipes.s_Alistipes_{—}onderdonkii | | | |
| k_Bacteria.p_Firmicutes.c_Clostridia.o_Clostridiales. | Beneficial_2 | 3.470678 | 7.34E−43 |
| f_Lachnospiraceae.g_Blautia.s_Ruminococcus_{—}gnavus | | | |
| k_Bacteria.p_Proteobacteria | Beneficial_2 | 3.276964 | 1.02E−18 |
| k_Bacteria.p_Firmicutes.c_Bacilli.o_Lactobacillies | Beneficial_2 | 3.65973 | 2.31E−79 |
| k_Bacteria.p_Actinobacteria.c_Bifidobacteriales. | Beneficial_2 | 3.689825 | 9.05E−11 |
| o_Bifidobacteriaceae.f_Bifidobacterium | | | |
| k_Bacteria.p_Actinobacteria.c_Bifidobacteriales. | Beneficial_2 | 3.689825 | 9.05E−11 |
| o_Bifidobacteriaceae.f_Bifidobacterium.g_Bifidobacterium | | | |
| k_Bacteria.p_Actinobacteria.c_Bifidobacteriales. | Beneficial_2 | 3.691189 | 4.16E−12 |
| o_Bifidobacteriaceae | | | |
| k_Bacteria.p_Actinobacteria.c_Bifidobacteriales | Beneficial_2 | 3.691189 | 4.16E−12 |
| k_Bacteria.p_Bacteroidetes.c_Bacteroidia.o_Bacteroidales. | Beneficial_2 | 3.755555 | 5.81E−12 |
| f_Bacteroidaceae.g_Bacteroides.s_Bacteroides_{—}thetaiotaomicron | | | |
| k_Bacteria.p_Bacteroidetes.c_Bacteroidia.o_Bacteroidales. | Beneficial_2 | 3.729009 | 1.18E−08 |
| f_Bacteroidaceae.g_Bacteroides.s_Bacteroides_{—}caccae | | | |
| k_Bacteria.p_Firmicutes.c_Clostridia.o_Clostridiales. | Beneficial_2 | 3.701306 | 1.14E−29 |
| f_Lachnospiraceae.g_Blautia.s_Blautia_{—}wexlerae | | | |
| k_Bacteria.p_Bacteroidetes.c_Bacteroidia.o_Bacteroidales. | Beneficial_2 | 3.89098 | 3.35E−69 |
| f_Bacteroidaceae.g_Bacteroides.s_Bacteroides_{—}fragilis | | | |
| k_Bacteria.p_Firmicutes.c_Erysipelotrichia | Beneficial_2 | 3.759608 | 1.45E−104 |
| k_Bacteria.p_Firmicutes.c_Erysipelotrichia. | Beneficial_2 | 3.759608 | 1.45E−104 |
| o_Erysipelotrichales.f_Erysipelotrichaceae | | | |
| k_Bacteria.p_Firmicutes.c_Erysipelotrichia. | Beneficial_2 | 3.759608 | 1.45E−104 |
| o_Erysipelotrichales | | | |
| k_Bacteria.p_Actinobacteria | Beneficial_2 | 3.819244 | 3.29E−39 |
| k_Bacteria.p_Firmicutes.c_Bacilli | Beneficial_2 | 3.936261 | 1.66E−128 |
| k_Bacteria.p_Verrucomicrobia.c_Verrucomicrobiae. | Beneficial_2 | 4.027925 | 2.13E−18 |
| o_Verrucomicrobiales.f_Akkermansiaceae.g_Akkermansia. | | | |
| s_Akkermansia_{—}muciniphila | | | |
| k_Bacteria.p_Verrucomicrobia.c_Verrucomicrobiae. | Beneficial_2 | 4.027925 | 2.13E−18 |
| o_Verrucomicrobiales.f_Akkermansiaceae | | | |
| k_Bacteria.p_Verrucomicrobia.c_Verrucomicrobiae. | Beneficial_2 | 4.027925 | 2.13E−18 |
| o_Verrucomicrobiales.f_Akkermansiaceae.g_Akkermansia | | | |
| k_Bacteria.p_Verrucomicrobia.c_Verrucomicrobiae. | Beneficial_2 | 4.028225 | 1.54E−18 |
| o_Verrucomicrobiales | | | |
| k_Bacteria.p_Verrucomicrobia | Beneficial_2 | 4.028225 | 1.54E−18 |
| k_Bacteria.p_Verrucomicrobia.c_Verrucomicrobiae | Beneficial_2 | 4.028225 | 1.54E−18 |
| k_Bacteria.p_Bacteroidetes.c_Bacteroidia.o_Bacteroidales. | Beneficial_2 | 3.812452 | 4.35E−27 |
| f_Porphyromonadaceae.g_Parabacteroides | | | |
| k_Bacteria.p_Bacteroidetes.c_Bacteroidia.o_Bacteroidales. | Beneficial_2 | 4.159305 | 1.37E−52 |
| f_Bacteroidaceae.g_Bacteroides.s_Bacteroides_{—}acidifaciens | | | |
| k_Bacteria.p_Bacteroidetes.c_Bacteroidia.o_Bacteroidales. | Beneficial_2 | 3.926018 | 3.30E−27 |
| f_Porphyromonadaceae | | | |
| k_Bacteria.p_Bacteroidetes.c_Bacteroidia.o_Bacteroidales. | Beneficial_2 | 4.172781 | 2.61E−26 |
| f_Bacteroidaceae.g_Bacteroides.s_Bacteroides_{—}dorei | | | |
| k_Bacteria.p_Firmicutes.c_Clostridia.o_Clostridiales. | Beneficial_2 | 4.193999 | 9.72E−135 |
| f_Lachnospiraceae.g_Blautia | | | |
| k_Bacteria.p_Firmicutes.c_Clostridia.o_Clostridiales. | Beneficial_2 | 4.415557 | 1.19E−84 |
| f_Lachnospiraceae | | | |
| k_Bacteria.p_Bacteroidetes.c_Bacteroidia.o_Bacteroidales. | Beneficial_2 | 4.702423 | 4.08E−77 |
| f_Bacteroidaceae | | | |
| k_Bacteria.p_Bacteroidetes.c_Bacteroidia.o_Bacteroidales. | Beneficial_2 | 4.702423 | 4.08E−77 |
| f_Bacteroidaceae.g_Bacteroides | | | |

### REFERENCES

1. Tumeh, P. C., et al. PD-1 blockade induces responses by inhibiting adaptive immune resistance. Nature 515, 568-571 (2014).
2. Giraldo, N. A., et al. Tumor-Infiltrating and Peripheral Blood T-cell Immunophenotypes Predict Early Relapse in Localized Clear Cell Renal Cell Carcinoma. Clin Cancer Res 23, 4416-4428 (2017).
3. Ballot, E., Ladoire, S., Routy, B., Truntzer, C. & Ghiringhelli, F. Tumor Infiltrating Lymphocytes Signature as a New Pan-Cancer Predictive Biomarker of Anti PD-1/PD-L1 Efficacy. Cancers (Basel) 12(2020).
4. Terranova-Barberio, M., et al. Exhausted T cell signature predicts immunotherapy response in ER-positive breast cancer. Nat Commun 11, 3584 (2020).
5. Taube, J. M., et al. Association of PD-1, PD-1 ligands, and other features of the tumor immune microenvironment with response to anti-PD-1 therapy. Clin Cancer Res 20, 5064-5074 (2014).
6. Herbst, R. S., et al. Predictive correlates of response to the anti-PD-L1 antibody MPDL3280A in cancer patients. Nature 515, 563-567 (2014).
7. Cristescu, R., et al. Pan-tumor genomic biomarkers for PD-1 checkpoint blockade-based immunotherapy. Science (New York, N.Y.) 362, eaar3593 (2018).
8. Rizvi, H., et al. Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol 36, 633-641 (2018).
9. Samstein, R. M., et al. Tumor mutational load predicts survival after immunotherapy across multiple cancer types. Nat Genet 51, 202-206 (2019).
10. Rizvi, N. A., et al. Cancer immunology. Mutational landscape determines sensitivity to PD-1 blockade in non-small cell lung cancer. Science 348, 124-128 (2015).
11. Huang, A. C., et al. A single dose of neoadjuvant PD-1 blockade predicts clinical outcomes in resectable melanoma. Nat Med 25, 454-461 (2019).
12. Huang, A. C., et al. T-cell invigoration to tumour burden ratio associated with anti-PD-1 response. Nature 545, 60-65 (2017).
13. Fairfax, B. P., et al. Peripheral CD8(+) T cell characteristics associated with durable responses to immune checkpoint blockade in patients with metastatic melanoma. Nat Med 26, 193-199 (2020).
14. Valpione, S., et al. Immune-awakening revealed by peripheral T cell dynamics after one cycle of immunotherapy. Nat Cancer 1, 210-221 (2020).
15. Wu, T. D., et al. Peripheral T cell expansion predicts tumour infiltration and clinical response. Nature 579, 274-278 (2020).
16. Sivan, A., et al. Commensal Bifidobacterium promotes antitumor immunity and facilitates anti-PD-L1 efficacy. Science 350, 1084-1089 (2015).
17. Vetizou, M., et al. Anticancer immunotherapy by CTLA-4 blockade relies on the gut microbiota. Science 350, 1079-1084 (2015).
18. Dzutsev, A., Goldszmid, R. S., Viaud, S., Zitvogel, L. & Trinchieri, G. The role of the microbiota in inflammation, carcinogenesis, and cancer therapy. Eur J Immunol 45, 17-31 (2015).
19. Gopalakrishnan, V., et al. Gut microbiome modulates response to anti-PD-1 immunotherapy in melanoma patients. Science 359, 97-103 (2018).
20. Matson, V., et al. The commensal microbiome is associated with anti-PD-1 efficacy in metastatic melanoma patients. Science 359, 104-10⁸ (2018).
21. Routy, B., et al. Gut microbiome influences efficacy of PD-1-based immunotherapy against epithelial tumors. Science 359, 91-97 (2018).
22. Baruch, E. N., et al. Fecal microbiota transplant promotes response in immunotherapy1298 refractory melanoma patients. Science 371, 602-609 (2021).
23. Davar, D., et al. Fecal microbiota transplant overcomes resistance to anti-PD-1 therapy in melanoma patients. Science 371, 595-602 (2021).
24. Frankel, A. E., et al. Metagenomic Shotgun Sequencing and Unbiased Metabolomic Profiling Identify Specific Human Gut Microbiota and Metabolites Associated with Immune Checkpoint Therapy Efficacy in Melanoma Patients. Neoplasia 19, 848-855 (2017).
25. Peters, B. A., et al. Relating the gut metagenome and metatranscriptome to immunotherapy responses in melanoma patients. Genome medicine 11, 61 (2019).
26. Shaikh, F. Y., et al. A uniform computational approach improved on existing pipelines to reveal microbiome biomarkers of non-response to immune checkpoint inhibitors. Clin Cancer Res (2021).
27. Gharaibeh, R. Z. & Jobin, C. Microbiota and cancer immunotherapy: in search of microbial signals. Gut 68, 385-388 (2019).
28. Limeta, A., Ji, B., Levin, M., Gatto, F. & Nielsen, J. Meta-analysis of the gut microbiota in predicting response to cancer immunotherapy in metastatic melanoma. JCI Insight 5(2020).
29. Chaput, N., et al. Baseline gut microbiota predicts clinical response and colitis in metastatic melanoma patients treated with ipilimumab. Ann Oncol 28, 1368-1379 (2017).
30. Dubin, K., et al. Intestinal microbiome analyses identify melanoma patients at risk for checkpoint-blockade-induced colitis. Nat Commun 7, 10391 (2016).
31. Wang, Y., et al. Fecal microbiota transplantation for refractory immune checkpoint inhibitor-associated colitis. Nat Med 24, 1804-1808 (2018).
32. Derosa, L., et al. Gut Bacteria Composition Drives Primary Resistance to Cancer Immunotherapy in Renal Cell Carcinoma Patients. European urology 78, 195-206 (2020).
33. Viaud, S., et al. The intestinal microbiota modulates the anticancer immune effects of cyclophosphamide. Science 342, 971-976 (2013).
34. Davar, D., et al. Phase Ib/II Study of Pembrolizumab and Pegylated-Interferon Alfa-2b in Advanced Melanoma. J Clin Oncol, JCO1800632 (2018).
35. Eisenhauer, E. A., et al. New response evaluation criteria in solid tumours: revised RECIST guideline (version 1.1). Eur J Cancer 45, 228-247 (2009).
36. Ogluszka, M., Orzechowska, M., Jedroszka, D., Witas, P. & Bednarek, A. K. Evaluate Cutpoints: Adaptable continuous data distribution system for determining survival in Kaplan-Meier estimator. Comput Methods Programs Biomed 177, 133-139 (2019).
37. Capone, M., et al. Baseline neutrophil-to-lymphocyte ratio (NLR) and derived NLR could predict overall survival in patients with advanced melanoma treated with nivolumab. Journal for immunotherapy of cancer 6, 74 (2018).
38. Valero, C., et al. Pretreatment neutrophil-to-lymphocyte ratio and mutational burden as biomarkers of tumor response to immune checkpoint inhibitors. Nat Commun 12, 729 (2021).
39. Ascierto, P. A., et al. Proteomic test for anti-PD-1 checkpoint blockade treatment of metastatic melanoma with and without BRAF mutations. J Immunother Cancer 7, 91 (2019).
40. Knight, J. M., et al. Non-invasive analysis of intestinal development in preterm and term infants using RNA-Sequencing. Sci Rep 4, 5453 (2014).
41. Rodrigues, R. R., Shulzhenko, N. & Morgun, A. Transkingdom Networks: A Systems Biology Approach to Identify Causal Members of Host-Microbiota Interactions. Methods Mol Biol 1849, 227-242 (2018).
42. Yambartsev, A., et al. Unexpected links reflect the noise in networks. Biol Direct 11, 52 (2016).
43. Das, S., et al. Immune-Related Adverse Events and Immune Checkpoint Inhibitor Efficacy in Patients with Gastrointestinal Cancer with Food and Drug Administration-Approved Indications for Immunotherapy. Oncologist 25, 669-679 (2020).
44. Matsuoka, H., et al. Correlation between immune-related adverse events and prognosis in patients with various cancers treated with anti PD-1 antibody. BMC Cancer 20, 656 (2020).
45. Suo, A., et al. Anti-PD1-Induced Immune-Related Adverse Events and Survival Outcomes in Advanced Melanoma. Oncologist 25, 438-446 (2020).
46. Horvath, A., et al. Biomarkers for oralization during long-term proton pump inhibitor therapy predict survival in cirrhosis. Sci Rep 9, 12000 (2019).
47. Cortellini, A., et al. Integrated analysis of concomitant medications and oncological outcomes from PD-1/PD-L1 checkpoint inhibitors in clinical practice. Journal for immunotherapy of cancer 8(2020).
48. Stacy, A., et al. Infection trains the host for microbiota-enhanced resistance to pathogens. Cell 184, 615-627.e617 (2021).
49. Kelsey, C. M., et al. Gut microbiota composition is associated with newborn functional brain connectivity and behavioral temperament. Brain Behav Immun 91, 472-486 (2021).
50. Benito, M., et al. Adjustment of systematic microarray data biases. Bioinformatics 20, 105-114 (2004).
51. Lazar, C., et al. Batch effect removal methods for microarray gene expression data integration: a survey. Brief Bioinform 14, 469-490 (2013).
52. Segata, N., et al. Metagenomic biomarker discovery and explanation. Genome biology 12, R60 (2011).
53. Kang, D. D., Froula, J., Egan, R. & Wang, Z. MetaBAT, an efficient tool for accurately reconstructing single genomes from complex microbial communities. PeerJ 3, e1165 (2015).
54. Kang, D. D., et al. MetaBAT 2: an adaptive binning algorithm for robust and efficient genome reconstruction from metagenome assemblies. PeerJ 7, e7359 (2019).
55. Balomenou, S., et al. Distinct functions of polysaccharide deacetylases in cell shape, neutral polysaccharide synthesis and virulence of Bacillus anthracis. Mol Microbiol 87, 867-883 (2013).
56. Bessman, N.J., et al. Dendritic cell-derived hepcidin sequesters iron from the microbiota to promote mucosal healing. Science 368, 186-189 (2020).
57. Crowther, M. D., et al. Genome-wide CRISPR-Cas9 screening reveals ubiquitous T cell cancer targeting via the monomorphic MHC class I-related protein MR1. Nat Immunol 21, 178-185 (2020).
58. Salio, M., et al. Ligand-dependent downregulation of MR1 cell surface expression. Proc Natl Acad Sci USA 117, 10465-10475 (2020).
59. Fan, S., et al. Cloning, characterization, and production of three alpha-L-fucosidases from Clostridium perfringens ATCC 13124. J Basic Microbiol 56, 347-357 (2016).
60. Wright, D. P., Rosendale, D. I. & Robertson, A. M. Prevotella enzymes involved in mucin oligosaccharide degradation and evidence for a small operon of genes expressed during growth on mucin. FEMS Microbiol Lett 190, 73-79 (2000).
61. Arumugam, M., et al. Enterotypes of the human gut microbiome. Nature 473, 174-180 (2011).
62. Brooks, A. W., Priya, S., Blekhman, R. & Bordenstein, S. R. Gut microbiota diversity across ethnicities in the United States. PLoS Biol 16, e2006842 (2018).
63. Dwiyanto, J., et al. Ethnicity influences the gut microbiota of individuals sharing a geographical location: a cross-sectional study from a middle-income country. Sci Rep 11, 2618 (2021).
64. Gorvitovskaia, A., Holmes, S. P. & Huse, S. M. Interpreting Prevotella and Bacteroides as biomarkers of diet and lifestyle. Microbiome 4, 15 (2016).
65. Knights, D., et al. Rethinking “enterotypes”. Cell Host Microbe 16, 433-437 (2014).
66. Zhang, R., Walker, A. R. & Datta, S. Unraveling city-specific signature and identifying sample origin locations for the data from CAMDA MetaSUB challenge. Biol Direct 16, 1 (2021).
67. McDonald, D., et al. American Gut: an Open Platform for Citizen Science Microbiome Research. mSystems 3(2018).
68. Levine, J. H., et al. Data-Driven Phenotypic Dissection of AML Reveals Progenitor-like Cells that Correlate with Prognosis. Cell 162, 184-197 (2015).
69. He, Y., et al. Regional variation limits applications of healthy gut microbiome reference ranges and disease models. Nat Med 24, 1532-1535 (2018).
70. Zarour, H. M. Reversing T-cell Dysfunction and Exhaustion in Cancer. Clin Cancer Res 22, 1856-1864 (2016).
71. Robert, C., et al. Durable Complete Response After Discontinuation of Pembrolizumab in Patients With Metastatic Melanoma. J Clin Oncol 36, 1668-1674 (2018).
72. Hamid, O., et al. Five-year survival outcomes for patients with advanced melanoma treated with pembrolizumab in KEYNOTE-001. Ann Oncol 30, 582-588 (2019).
73. Luke, N. R., et al. Identification and characterization of a glycosyltransferase involved in Acinetobacter baumannii lipopolysaccharide core biosynthesis. Infect Immun 78, 2017-2023 (2010).
74. Alfaro, C., et al. Tumor-Produced Interleukin-8 Attracts Human Myeloid-Derived Suppressor Cells and Elicits Extrusion of Neutrophil Extracellular Traps (NETs). Clin Cancer Res 22, 3924-3936 (2016).
75. Sanmamed, M. F., et al. Changes in serum interleukin-8 (IL-8) levels reflect and predict response to anti-PD-1 treatment in melanoma and non-small-cell lung cancer patients. Ann Oncol 28, 1988-1995 (2017).
76. Schalper, K. A., et al. Elevated serum interleukin-8 is associated with enhanced intratumor neutrophils and reduced clinical benefit of immune-checkpoint inhibitors. Nat Med 26, 688-692 (2020).
77. Kaplanov, I., et al. Blocking IL-1beta reverses the immunosuppression in mouse breast cancer and synergizes with anti-PD-1 for tumor abrogation. Proc Natl Acad Sci USA 116, 1361-1369 (2019).
78. Bertrand, F., et al. TNFalpha blockade overcomes resistance to anti-PD-1 in experimental melanoma. Nat Commun 8, 2256 (2017).
79. Freeman-Keller, M., et al. Nivolumab in Resected and Unresectable Metastatic Melanoma: Characteristics of Immune-Related Adverse Events and Association with Outcomes. Clin Cancer Res 22, 886-894 (2016).
80. Weber, J. S., et al. Safety Profile of Nivolumab Monotherapy: A Pooled Analysis of Patients With Advanced Melanoma. J Clin Oncol 35, 785-792 (2017).
81. Weber, J. S., Kahler, K. C. & Hauschild, A. Management of immune-related adverse events and kinetics of response with ipilimumab. J Clin Oncol 30, 2691-2697 (2012).
82. Murillo, O., et al. Streptococcal vertebral osteomyelitis: multiple faces of the same disease. Clin Microbiol Infect 20, O33-38 (2014).
83. Boer, C. G., et al. Intestinal microbiome composition and its relation to joint pain and inflammation. Nat Commun 10, 4881 (2019).
84. Cunningham, M. W. Rheumatic fever, autoimmunity, and molecular mimicry: the streptococcal connection. Int Rev Immunol 33, 314-329 (2014).
85. Tandon, R., et al. Revisiting the pathogenesis of rheumatic fever and carditis. Nat Rev Cardiol 10, 171-177 (2013).
86. Barash, J. Rheumatic Fever and post-group a streptococcal arthritis in children. Curr Infect Dis Rep 15, 263-268 (2013).
87. McCarville, J. L. & Ayres, J. S. Disease tolerance: concept and mechanisms. Curr Opin Immunol 50, 88-93 (2018).
88. Rose, N. R. Negative selection, epitope mimicry and autoimmunity. Curr Opin Immunol 49, 51-55 (2017). 89. Yumoto, H., et al. The Pathogenic Factors from Oral Streptococci for Systemic Diseases. Int J Mol Sci 20(2019).
90. Gevers, D., et al. The Human Microbiome Project: a community resource for the healthy human microbiome. PLoS Biol 10, e1001377 (2012).
91. Human Microbiome Project, C. Structure, function and diversity of the healthy human microbiome. Nature 486, 207-214 (2012).
92. Human Microbiome Project, C. A framework for human microbiome research. Nature 486, 215-221 (2012).
93. Asnicar, F., et al. Microbiome connections with host metabolism and habitual diet from 1,098 deeply phenotyped individuals. Nat Med 27, 321-332 (2021).
94. Costea, P. I., et al. Enterotypes in the landscape of gut microbial community composition. Nat Microbiol 3, 8-16 (2018).
95. Tjalsma, H., Boleij, A., Marchesi, J. R. & Dutilh, B. E. A bacterial driver-passenger model for colorectal cancer: beyond the usual suspects. Nat Rev Microbiol 10, 575-582 (2012).
96. Bolger, A. M., Lohse, M. & Usadel, B. Trimmomatic: a flexible trimmer for Illumina sequence data. Bioinformatics 30, 2114-2120 (2014).
97. Langmead, B. & Salzberg, S. L. Fast gapped-read alignment with Bowtie 2. Nature methods 9, 357-359 (2012).
98. Li, D., Liu, C. M., Luo, R., Sadakane, K. & Lam, T. W. MEGAHIT: an ultra-fast single-node solution for large and complex metagenomics assembly via succinct de Bruijn graph. Bioinformatics 31, 1674-1676 (2015).
99. Li, D., et al. MEGAHIT v1.0: A fast and scalable metagenome assembler driven by advanced methodologies and community practices. Methods 102, 3-11 (2016).
100. Wood, D. E., Lu, J. & Langmead, B. Improved metagenomic analysis with Kraken 2. Genome Biol 20, 257 (2019).
101. Seemann, T. Prokka: rapid prokaryotic genome annotation. Bioinformatics 30, 2068-2069 (2014).
102. Gu, Z., Eils, R. & Schlesner, M. Complex heatmaps reveal patterns and correlations in multidimensional genomic data. Bioinformatics 32, 2847-2849 (2016).
103. Shannon, P., et al. Cytoscape: a software environment for integrated models of biomolecular interaction networks. Genome Res 13, 2498-2504 (2003).
104. Johnson, W. E., Li, C. & Rabinovic, A. Adjusting batch effects in microarray expression data using empirical Bayes methods. Biostatistics 8, 118-127 (2007).
105. Ho, T. K. Random decision forests. in Proceedings of the Third International Conference on Document Analysis and Recognition (Volume 1)—Volume 1 278 (IEEE Computer Society, 1995).
106. Boser, BE., Guyon, I. M. & Vapnik, V. N. A training algorithm for optimal margin classifiers. in Proceedings of the fifth annual workshop on Computational learning theory 144-152 (Association for Computing Machinery, Pittsburgh, Pennsylvania, USA, 1992).
107. Nelder, P. M. J. A. Generalized Linear Models, (Chapman and Hall/CRC, 1989).
108. Vinayak, R. K. & Gilad-Bachrach, R. DART: Dropouts meet Multiple Additive Regression Trees. in Proceedings of the Eighteenth International Conference on Artificial Intelligence and Statistics, Vol. 38 (eds. Guy, L. & Vishwanathan, S. V. N.) 489-497 (PMLR, Proceedings of Machine Learning Research, 2015).

1. A method of increasing effectiveness of an immune checkpoint inhibitor in a subject comprising administering to the subject the immune checkpoint inhibitor and increasing an amount of one or more bacteria in an intestine of the subject to a therapeutically effective amount, wherein the one or more bacteria are selected from the group consisting of Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Enterocloster clostridioformis, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Enterocloster lavalensis, Faecalibacterium prausnitzii, Eubacterium limosum, an Anaerotruncus Unclassified, Duodenibacillus massiliensis, Faecalimonas umbilicata, Absiella dolichum, Prevotella oryzae, Prevotella lascolaii, Prevotella, Prevotella koreensis, Oxalobacter formigenes, Prevotella stercorea, a Prevotella Unclassified, Prevotella copri, Prevotella buccae, Bacteroides caccae, Lactobacillus paracasei, Acinetobacter baumannii, Acidaminococcus fermentans, Lactobacillus rhamnosus, a Bacillus Unclassified, and a Faecalibacterium Unclassified.

2. The method of claim 1, wherein the one or more bacteria are selected from a Lachnospiraceae spp.

3. (canceled)

4. The method of claim 1, wherein the one or more bacteria are administered to the subject.

5. The method of claim 4, wherein the administration of the one or more bacteria occurs prior to the administration of the immune checkpoint inhibitor.

6. The method of claim 4, wherein the administration of the one or more bacteria occurs after the administration of the immune checkpoint inhibitor.

7. A method of increasing effectiveness of an immune checkpoint inhibitor in a subject comprising administering to the subject the immune checkpoint inhibitor and decreasing an amount of one or more bacteria in an intestine of the subject, wherein the one or more bacteria are selected from the group consisting of Prevotella koreensis, Prevotella oryzae, Intestinimonas butyriciproducens, Bacteroidales bacterium, Alistipes senegalensis, a Subdoligranulum Unclassified, Sporobacter termitidis, a Streptomyces Unclassified, Pseudoflavonifractor, Clostridium perfringens, Veillonella parvula, Oscillibacter ruminantium, a Prevotella Unclassified, Haemophilus, Lactobacillus fermentum, Proteobacteria, Streptococcus mutans, Ruminococcus flavefaciens, Prevotella buccae, Eubacterium pyruvativorans, Oscillibacter valericigenes, Bacillus, Cuneatibacter caecimuris, Acinetobacter baumannii, Actinobacteria, Porphyromonas uenonis, Alistipes dispar, Christensenella minuta, Veillonella, Streptococcus vestibularis, Fournierella massiliensis, Eubacterium siraeum, Agathobaculum desmolans, Oxalobacter formigenes, Bacteroidales, Victivallales bacterium, Alistipes finegoldii, Bacteroides ilei, Intestinimonas massiliensis, Lachnospira eligens, a Bacteriophage Unclassified, Odoribacter splanchnicus, a Bacillus Unclassified, Angelakisella massiliensis, Haemophilus parainfluenzae, Ruminococcus bromii, a Flintibacter Unclassified, Sutterellaceae, Alistipes shahii, Bacteroides caccae, Odoribacter laneus, Alistipes communis, Acidaminococcus, Rikenellaceae bacterium, Klebsiella, Bacteroides faecis, Coprobacter fastidiosus, Collinsella, a Paenibacillus Unclassified, a Pseudoflavonifractor Unclassified, Streptococcus, a Faecalibacterium Unclassified, Ruminococcus albus, Eukaryota, Alistipes timonensis, an Oscillibacter Unclassified, Lactobacillus salivarius, Intestinibacillus massiliensis, a Haemophilus Unclassified, Acidaminococcus fermentans, Streptococcus salivarius, a Muribaculum Unclassified, Clostridiales bacterium, Lactobacillus, Flavonifractor plautii, Faecalibacterium, Bacteroides massiliensis, Lactobacillus paracasei, Ruminococcus lactaris, Subdoligranulum, Anaerotruncus massiliensis, Ruminococcus champanellensis, Enterobacter hormaechei, Clostridium, Cyclospora cayetanensis, Anaeromassilibacillus senegalensis, Bacteroides ovatus, Butyricimonas virosa, Ruthenibacterium lactatiformans, Bacteroides finegoldii, Alistipes putredinis, a Butyricicoccus Unclassified, Bacteroides cellulosilyticus, Bacteroides coprocola, Intestinimonas timonensis, Victivallis vadensis, Sutterella wadsworthensis, Clostridium botulinum, Coprococcus eutactus, Anaerotignum lactatifermentans, a Duncaniella Unclassified, Escherichia, an Acidaminococcus Unclassified, Dysosmobacter welbionis, Butyricimonas, Bacteroides intestinalis, Bilophila wadsworthia, Desulfovibrio fairfieldensis, Lactobacillus gasseri, Agathobaculum butyriciproducens, Klebsiella pneumoniae, Faecalitalea cylindroides, Porphyromonas asaccharolytica, Harryflintia acetispora, Butyricicoccus pullicaecorum, Lactobacillus paragasseri, Alistipes indistinctus, an Akkermansia Unclassified, Cryptobacterium curtum, Bacteroides vulgatus, Oscillibacter, a Prevotella Unclassified, Prevotella, Butyricimonas virosa, Subdoligranulum, Bacteroides massiliensis, Prevotella copri, Enterocloster clostridioformis, Prevotella stercorea, Prevotella lascolaii, Desulfovibrio fairfieldensis, Anaerotruncus colihominis, Streptococcus parasanguinis, Eubacterium maltosivorans, Bacteroides plebeius, a Megasphaera Unclassified, a Desulfovibrio Unclassified, Escherichia coli, Desulfovibrio desulfuricans, Enterobacteriaceae, Parabacteroides merdae, Akkermansia muciniphila, Paraprevotella clara, Enterocloster lavalensis, and Dialister invisus.

8. The method of claim 7, wherein the one or more bacteria are selected from a Streptococcus spp.

9. (canceled)

10. A method of treating a cancer in a subject comprising increasing an amount of one or more bacteria selected to a therapeutically effective amount, wherein the one or more bacterial are selected from the group consisting of Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Enterocloster clostridioformis, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Enterocloster lavalensis, Faecalibacterium prausnitzii, Eubacterium limosum, an Anaerotruncus Unclassified, Duodenibacillus massiliensis, Faecalimonas umbilicata, Absiella dolichum, Prevotella oryzae, Prevotella lascolaii, Prevotella, Prevotella koreensis, Oxalobacter formigenes, Prevotella stercorea, a Prevotella Unclassified, Prevotella copri, Prevotella buccae, Bacteroides caccae, Lactobacillus paracasei, Acinetobacter baumannii, Acidaminococcus fermentans, Lactobacillus rhamnosus, a Bacillus Unclassified, and a Faecalibacterium Unclassified, and wherein the subject receives an immune checkpoint inhibitor.

11. The method of claim 10, wherein the one or more bacteria are selected from a Lachnospiraceae spp.

12. (canceled)

13. The method of claim 10, wherein the subject is administered a therapeutically effective amount of the one or more bacteria.

14. The method of claim 13, wherein the administration of the one or more bacteria occurs prior to the administration to the subject of the immune checkpoint inhibitor.

15. The method of claim 13, wherein the administration of the one or more bacteria occurs after the administration to the subject of the immune checkpoint inhibitor.

16. The method of claim 10, further comprising decreasing an amount of one or more bacteria selected from the group consisting of Prevotella koreensis, Prevotella oryzae, Intestinimonas butyriciproducens, Bacteroidales bacterium, Alistipes senegalensis, a Subdoligranulum Unclassified, Sporobacter termitidis, a Streptomyces Unclassified, Pseudoflavonifractor, Clostridium perfringens, Veillonella parvula, Oscillibacter ruminantium, a Prevotella Unclassified, Haemophilus, Lactobacillus fermentum, Proteobacteria, Streptococcus mutans, Ruminococcus flavefaciens, Prevotella buccae, Eubacterium pyruvativorans, Oscillibacter valericigenes, Bacillus, Cuneatibacter caecimuris, Acinetobacter baumannii, Actinobacteria, Porphyromonas uenonis, Alistipes dispar, Christensenella minuta, Veillonella, Streptococcus vestibularis, Fournierella massiliensis, Eubacterium siraeum, Agathobaculum desmolans, Oxalobacter formigenes, Bacteroidales, Victivallales bacterium, Alistipes finegoldii, Bacteroides ilei, Intestinimonas massiliensis, Lachnospira eligens, a Bacteriophage Unclassified, Odoribacter splanchnicus, a Bacillus Unclassified, Angelakisella massiliensis, Haemophilus parainfluenzae, Ruminococcus bromii, a Flintibacter Unclassified, Sutterellaceae, Alistipes shahii, Bacteroides caccae, Odoribacter laneus, Alistipes communis, Acidaminococcus, Rikenellaceae bacterium, Klebsiella, Bacteroides faecis, Coprobacter fastidiosus, Collinsella, a Paenibacillus Unclassified, a Pseudoflavonifractor Unclassified, Streptococcus, a Faecalibacterium Unclassified, Ruminococcus albus, Eukaryota, Alistipes timonensis, an Oscillibacter Unclassified, Lactobacillus salivarius, Intestinibacillus massiliensis, a Haemophilus Unclassified, Acidaminococcus fermentans, Streptococcus salivarius, a Muribaculum Unclassified, Clostridiales bacterium, Lactobacillus, Flavonifractor plautii, Faecalibacterium, Bacteroides massiliensis, Lactobacillus paracasei, Ruminococcus lactaris, Subdoligranulum, Anaerotruncus massiliensis, Ruminococcus champanellensis, Enterobacter hormaechei, Clostridium, Cyclospora cayetanensis, Anaeromassilibacillus senegalensis, Bacteroides ovatus, Butyricimonas virosa, Ruthenibacterium lactatiformans, Bacteroides finegoldii, Alistipes putredinis, a Butyricicoccus Unclassified, Bacteroides cellulosilyticus, Bacteroides coprocola, Intestinimonas timonensis, Victivallis vadensis, Sutterella wadsworthensis, Clostridium botulinum, Coprococcus eutactus, Anaerotignum lactatifermentans, a Duncaniella Unclassified, Escherichia, an Acidaminococcus Unclassified, Dysosmobacter welbionis, Butyricimonas, Bacteroides intestinalis, Bilophila wadsworthia, Desulfovibrio fairfieldensis, Lactobacillus gasseri, Agathobaculum butyriciproducens, Klebsiella pneumoniae, Faecalitalea cylindroides, Porphyromonas asaccharolytica, Harryflintia acetispora, Butyricicoccus pullicaecorum, Lactobacillus paragasseri, Alistipes indistinctus, an Akkermansia Unclassified, Cryptobacterium curtum, Bacteroides vulgatus, Oscillibacter, a Prevotella Unclassified, Prevotella, Butyricimonas virosa, Subdoligranulum, Bacteroides massiliensis, Prevotella copri, Enterocloster clostridioformis, Prevotella stercorea, Prevotella lascolaii, Desulfovibrio fairfieldensis, Anaerotruncus colihominis, Streptococcus parasanguinis, Eubacterium maltosivorans, Bacteroides plebeius, a Megasphaera Unclassified, a Desulfovibrio Unclassified, Escherichia coli, Desulfovibrio desulfuricans, Enterobacteriaceae, Parabacteroides merdae, Akkermansia muciniphila, Paraprevotella clara, Enterocloster lavalensis, and Dialister invisus.

17. The method of claim 16, wherein the one or more bacteria are selected from a Streptococcus spp.

18. (canceled)

19. A method of predicting a subject's responsiveness to an immune checkpoint inhibitor comprising obtaining a stool sample from the subject and determining the presence or absence of one or more bacteria in the sample, wherein the one or more bacteria are selected from the group consisting of Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, a Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Faecalibacterium prausnitzii, Eubacterium limosum, an Anaerotruncus Unclassified, Duodenibacillus massiliensis, Faecalimonas umbilicata, Absiella dolichum, Bacteria, Lactobacillus rhamnosus, Prevotella koreensis, Prevotella oryzae, Intestinimonas butyriciproducens, Bacteroidales bacterium, Alistipes senegalensis, a Subdoligranulum Unclassified, Sporobacter termitidis, a Streptomyces Unclassified, Pseudoflavonifractor, Clostridium perfringens, Veillonella parvula, Oscillibacter ruminantium, Haemophilus, Lactobacillus fermentum, Proteobacteria, Streptococcus mutans, Ruminococcus flavefaciens, Prevotella buccae, Eubacterium pyruvativorans, Oscillibacter valericigenes, Bacillus, Cuneatibacter caecimuris, Acinetobacter baumannii, Actinobacteria, Porphyromonas uenonis, Alistipes dispar, Christensenella minuta, Veillonella, Streptococcus vestibularis, Fournierella massiliensis, Eubacterium siraeum, Agathobaculum desmolans, Oxalobacter formigenes, Bacteroidales, Victivallales bacterium, Alistipes finegoldii, Bacteroides ilei, Intestinimonas massiliensis, Lachnospira eligens, a Bacteriophage Unclassified, Odoribacter splanchnicus, a Bacillus Unclassified, Angelakisella massiliensis, Haemophilus parainfluenzae, Ruminococcus bromii, a Flintibacter Unclassified, Sutterellaceae, Alistipes shahii, Bacteroides caccae, Odoribacter laneus, Alistipes communis, Acidaminococcus, Rikenellaceae bacterium, Klebsiella, Bacteroides faecis, Coprobacter fastidiosus, Collinsella, a Paenibacillus Unclassified, Pseudoflavonifractor Unclassified, Streptococcus, Faecalibacterium Unclassified, Ruminococcus albus, Eukaryota, Alistipes timonensis, an Oscillibacter Unclassified, Lactobacillus salivarius, Intestinibacillus massiliensis, a Haemophilus Unclassified, Acidaminococcus fermentans, Streptococcus salivarius, a Muribaculum Unclassified, Clostridiales bacterium, Lactobacillus, Flavonifractor plautii, Faecalibacterium, Lactobacillus paracasei, Ruminococcus lactaris, Anaerotruncus massiliensis, Ruminococcus champanellensis, Enterobacter hormaechei, Clostridium, Cyclospora cayetanensis, Anaeromassilibacillus senegalensis, Bacteroides ovatus, Ruthenibacterium lactatiformans, Bacteroides finegoldii, Alistipes putredinis, a Butyricicoccus Unclassified, Bacteroides cellulosilyticus, Bacteroides coprocola, Intestinimonas timonensis, Victivallis vadensis, Sutterella wadsworthensis, Clostridium botulinum, Coprococcus eutactus, Anaerotignum lactatifermentans, a Duncaniella Unclassified, Escherichia, an Acidaminococcus Unclassified, Dysosmobacter welbionis, Butyricimonas, Bacteroides intestinalis, Bilophila wadsworthia, Lactobacillus gasseri, Agathobaculum butyriciproducens, Klebsiella pneumoniae, Faecalitalea cylindroides, Porphyromonas asaccharolytica, Harryflintia acetispora, Butyricicoccus pullicaecorum, Lactobacillus paragasseri, Alistipes indistinctus, an Akkermansia Unclassified, Cryptobacterium curtum, Bacteroides vulgatus, Oscillibacter, Prevotella Unclassified, Prevotella, Butyricimonas virosa, Subdoligranulum, Bacteroides massiliensis, Prevotella copri, Enterocloster clostridioformis, Prevotella stercorea, Prevotella lascolaii, Desulfovibrio fairfieldensis, Anaerotruncus colihominis, Streptococcus parasanguinis, Eubacterium maltosivorans, Bacteroides plebeius, a Megasphaera Unclassified, a Desulfovibrio Unclassified, Escherichia coli, Desulfovibrio desulfuricans, Enterobacteriaceae, Parabacteroides merdae, Akkermansia muciniphila, Paraprevotella clara, Enterocloster lavalensis, and Dialister invisus;
wherein detection of the one or more bacteria selected from the group consisting of consisting of Blautia coccoides, Lachnospiraceae, Ruminococcus torques, Blautia producta, Enterocloster bolteae, Bifidobacterium bifidum, Erysipelotrichaceae, Anaerostipes hadrus, Lachnospiraceae bacterium, Clostridium scindens, Enterorhabdus caecimuris, Eisenbergiella massiliensis, Collinsella intestinalis, Actinomyces oris, Coprococcus, Enterocloster clostridioformis, Lactococcus lactis, Blautia hydrogenotrophica, Bacteroides eggerthii, Blautia hansenii, Candidatus Stoquefichus, Dorea longicatena, Coprococcus comes, Blautia, Blautia obeum, Firmicutes bacterium, Clostridiaceae bacterium, Bacteroides thetaiotaomicron, a Blautia Unclassified, Anaerostipes caccae, Adlercreutzia equolifaciens, Lactobacillus crispatus, Clostridium methylpentosum, Fusicatenibacter saccharivorans, an Adlercreutzia Unclassified, a Coprobacillus Unclassified, Enterocloster aldensis, Blautia wexlerae, Mogibacterium diversum, Streptococcus thermophilus, Parabacteroides johnsonii, Dorea, Dorea Unclassified, Eubacterium rectale, Gordonibacter urolithinfaciens, Schaalia odontolytica, Ruminococcus gnavus, Akkermansiaceae, Eggerthellaceae, Parabacteroides distasonis, Asaccharobacter celatus, Sellimonas intestinalis, Mogibacterium pumilum, Eggerthella lenta, Erysipelatoclostridium ramosum, Eubacteriaceae bacterium, Catenibacterium mitsuokai, Collinsella stercoris, Actinomyces bouchesdurhonensis, Anaerostipes, an Anaeromassilibacillus Unclassified, Tyzzerella nexilis, Blautia schinkii, Enterocloster lavalensis, Faecalibacterium prausnitzii, Eubacterium limosum, an Anaerotruncus Unclassified, Duodenibacillus massiliensis, Faecalimonas umbilicata, Absiella dolichum, Prevotella oryzae, Prevotella lascolaii, Prevotella, Prevotella koreensis, Oxalobacter formigenes, Prevotella stercorea, a Prevotella Unclassified, Prevotella copri, Prevotella buccae, Bacteroides caccae, Lactobacillus paracasei, Acinetobacter baumannii, Acidaminococcus fermentans, Lactobacillus rhamnosus, a Bacillus Unclassified, and a Faecalibacterium Unclassified predicts responsiveness;
and wherein detection of the one or more bacteria selected from the group consisting of Prevotella koreensis, Prevotella oryzae, Intestinimonas butyriciproducens, Bacteroidales bacterium, Alistipes senegalensis, a Subdoligranulum Unclassified, Sporobacter termitidis, a Streptomyces Unclassified, Pseudoflavonifractor, Clostridium perfringens, Veillonella parvula, Oscillibacter ruminantium, a Prevotella Unclassified, Haemophilus, Lactobacillus fermentum, Proteobacteria, Streptococcus mutans, Ruminococcus flavefaciens, Prevotella buccae, Eubacterium pyruvativorans, Oscillibacter valericigenes, Bacillus, Cuneatibacter caecimuris, Acinetobacter baumannii, Actinobacteria, Porphyromonas uenonis, Alistipes dispar, Christensenella minuta, Veillonella, Streptococcus vestibularis, Fournierella massiliensis, Eubacterium siraeum, Agathobaculum desmolans, Oxalobacter formigenes, Bacteroidales, Victivallales bacterium, Alistipes finegoldii, Bacteroides ilei, Intestinimonas massiliensis, Lachnospira eligens, a Bacteriophage Unclassified, Odoribacter splanchnicus, a Bacillus Unclassified, Angelakisella massiliensis, Haemophilus parainfluenzae, Ruminococcus bromii, a Flintibacter Unclassified, Sutterellaceae, Alistipes shahii, Bacteroides caccae, Odoribacter laneus, Alistipes communis, Acidaminococcus, Rikenellaceae bacterium, Klebsiella, Bacteroides faecis, Coprobacter fastidiosus, Collinsella, a Paenibacillus Unclassified, a Pseudoflavonifractor Unclassified, Streptococcus, a Faecalibacterium Unclassified, Ruminococcus albus, Eukaryota, Alistipes timonensis, an Oscillibacter Unclassified, Lactobacillus salivarius, Intestinibacillus massiliensis, a Haemophilus Unclassified, Acidaminococcus fermentans, Streptococcus salivarius, a Muribaculum Unclassified, Clostridiales bacterium, Lactobacillus, Flavonifractor plautii, Faecalibacterium, Bacteroides massiliensis, Lactobacillus paracasei, Ruminococcus lactaris, Subdoligranulum, Anaerotruncus massiliensis, Ruminococcus champanellensis, Enterobacter hormaechei, Clostridium, Cyclospora cayetanensis, Anaeromassilibacillus senegalensis, Bacteroides ovatus, Butyricimonas virosa, Ruthenibacterium lactatiformans, Bacteroides finegoldii, Alistipes putredinis, a Butyricicoccus Unclassified, Bacteroides cellulosilyticus, Bacteroides coprocola, Intestinimonas timonensis, Victivallis vadensis, Sutterella wadsworthensis, Clostridium botulinum, Coprococcus eutactus, Anaerotignum lactatifermentans, a Duncaniella Unclassified, Escherichia, an Acidaminococcus Unclassified, Dysosmobacter welbionis, Butyricimonas, Bacteroides intestinalis, Bilophila wadsworthia, Desulfovibrio fairfieldensis, Lactobacillus gasseri, Agathobaculum butyriciproducens, Klebsiella pneumoniae, Faecalitalea cylindroides, Porphyromonas asaccharolytica, Harryflintia acetispora, Butyricicoccus pullicaecorum, Lactobacillus paragasseri, Alistipes indistinctus, an Akkermansia Unclassified, Cryptobacterium curtum, Bacteroides vulgatus, Oscillibacter, a Prevotella Unclassified, Prevotella, Butyricimonas virosa, Subdoligranulum, Bacteroides massiliensis, Prevotella copri, Enterocloster clostridioformis, Prevotella stercorea, Prevotella lascolaii, Desulfovibrio fairfieldensis, Anaerotruncus colihominis, Streptococcus parasanguinis, Eubacterium maltosivorans, Bacteroides plebeius, a Megasphaera Unclassified, a Desulfovibrio Unclassified, Escherichia coli, Desulfovibrio desulfuricans, Enterobacteriaceae, Parabacteroides merdae, Akkermansia muciniphila, Paraprevotella clara, Enterocloster lavalensis, and Dialister invisus predicts lack of responsiveness.

20. (canceled)

21. (canceled)

22. The method of claim 19, wherein the one or more bacteria are selected from a Lachnospiraceae spp.

23. The method of claim 19, wherein the one or more bacteria are selected from the group consisting of Ruminococcus (Mediterraneibacter) torques, Ruminococcus (Mediterraneibacter) gnavus, Blautia wexlerae, Blautia hansenii, Eubacterium rectale, Actinomyces bouchesdurhonensis, Bacteroides massiliensis, Bacteroides stercoris, Prevotella copri, and Bacteroides plebeius.

24. (canceled)

25. (canceled)

26. The method of claim 1, wherein the immune checkpoint inhibitor is a PD-1 inhibitor or a PD-L1 inhibitor.

27. (canceled)

28. (canceled)

29. The method of claim 10, wherein the cancer is a melanoma.

30. The method of claim 10, wherein a tumor volume or amount is decreased as compared to a control.

31. The method of claim 10, wherein a metastasis or recurrence of the cancer is decreased as compared to a control.

32. The method of claim 1, wherein the subject is a human.
